(19) United States
(12) Patent Application Publication
(10) Pub. No.: US 2014/0030218 A1
(43) Pub. Date: Jan. 30, 2014

(54) Treatment And Screening

(71) Applicants: Irina Alexandrovna Udalova, Kingston upon Thames (GB); Thomas Krausgruber, Oxford (GB); Marc Feldmann, London (GB); David Saliba, London (GB); Hayley Eames, Reading (GB)

(72) Inventors: Irina Alexandrovna Udalova, Kingston upon Thames (GB); Thomas Krausgruber, Oxford (GB); Marc Feldmann, London (GB); David Saliba, London (GB); Hayley Eames, Reading (GB)

(73) Assignee: IMPERIAL INNOVATIONS LIMITED, South Kensington London (GB)

(21) Appl. No.: 13/978,504

(22) PCT Filed: Jan. 5, 2012
(86) PCT No.: PCT/GB12/50011

§ 371 Date: Oct. 18, 2013

Related U.S. Application Data
(60) Provisional application No. 61/429,877, filed on Jan. 5, 2011.

(51) Int. Cl. C12N 15/113

(52) U.S. Cl. CPC C12N 15/113
USPC 424/85.2; 424/85.1; 424/85.4; 435/7.1; 435/7.92; 514/1.9; 514/2.4; 514/3.4; 514/3.7; 514/4.6; 514/16.6; 514/17.9; 514/18.8; 514/19.3; 514/44 R; 514/44 A

(57) ABSTRACT

A method of treating a patient having an autoimmune disease or a Th1 polarising infection or a condition associated with inflammation other than asthma or allergy, the method comprising administering to the patient a therapeutically effective amount of an inhibitor of Interferon Regulatory Factor 5 (IRF5).

This invention relates to modulating the immune system, and in particular to methods for modulating the immune system to treat disease. The invention further relates to methods for identifying agents that modulate the immune system to treat disease.

The listing or discussion of a prior-published document in this specification should not necessarily be taken as an acknowledgement that the document is part of the state of the art or is common general knowledge. Any document referred to herein is hereby incorporated by reference.

Macrophages are a heterogeneous population of immune cells that are essential for the initiation and resolution of pathogen- or tissue damage-induced inflammation. They demonstrate remarkable plasticity that allows them to efficiently respond to environmental signals and change their phenotype and physiology in response to cytokines and microbial signals. These changes can give rise to populations of cells with distinct functions, which are phenotypically characterised by production of pro-inflammatory and anti-inflammatory cytokines. Based on the Th1/Th2 polarisation concept these cells are now referred to as M1 (classic) macrophages, that produce pro-inflammatory cytokines and mediate resistance to pathogens and tissue destruction, and M2 (alternative) macrophages, that produce anti-inflammatory cytokines and promote tissue repair and remodelling as well as tumour progression.

The activation of a subset-defining transcription factor is characteristic of a particular T cell lineage commitment: T-bet is associated with Th1, GATA3 with Th2, FOXP3 with Treg and RORγT with Th17 cells. Dendritic cells (DCs) also employ subset-selective expression of IRF4 and IRF8 for their commitment. IRF4 is expressed at high levels in CD4⁺ DCs but low in pDCs. As a consequence, the CD4⁺ DC population is absent in irf4^{-/-} mice. Conversely, IRF8 is expressed at high levels in pDCs and CD8⁺ DCs, thus irf8^{-/-} mice are largely devoid of these DC subsets. However, transcription factors underlying macrophage polarisation remain largely undefined.

Activation of NE-κB p50 has been previously associated with inhibition of M1 polarising genes (Porta et al, 2009), whereas CREB mediated induction of C/EBPβ has been shown to upregulate M2-specific genes (Ruffell et al, 2009). More recent evidence suggests that, in mice, IRF4 may control M2 macrophage polarisation by stimulating the expression of selected M2 macrophage markers (Satoh et al, 2010). However, no global determinant of M1 macrophage lineage commitment has been identified.

IRF5, a member of the IRF family, has diverse activities, such as activation of type I IFN genes, inflammatory cytokines, including TNF, IL-6, IL-12 and IL-23, and tumour suppressors (Ouyang et al, 2007), and IRF5 deficient mice are resistant to lethal endotoxic shock (Takaoka et al, 2005). Moreover, genetic polymorphism in the IRF5 gene, leading to expression of several unique isoforms or increased expression of IRF5 mRNA, is implicated in autoimmune diseases including systemic lupus erythematosus (SLE), rheumatoid arthritis (RA), Sjogren's syndrome, multiple sclerosis and inflammatory bowel disease. Nevertheless, the role of IRF5, and its mechanism of action, remains unclear.

The inventors have now shown that activation of IRF5 expression defines macrophage lineage commitment by driving M1 macrophage polarisation. High levels of IRF5 are characteristic of pro-inflammatory M1 (IL-12^{high}, IL-23^{high}, IL-10^{low}) macrophages, in which IRF5 directly activates transcription of IL-12p40/p35 and IL-23p19 and represses IL-10 genes.

More specifically, in Example 1 the inventors have shown that M1 macrophages are characterised by high level of IRF5, expression of which is induced during their differentiation with either GM-CSF or IFN-γ/LPS. Forced expression of IRF5 in M2 macrophages drives global expression of M1-specific cytokines, chemokines and co-stimulatory molecules and leads to a potent Th1/Th17 response. Conversely, the induction of IL-12, IL-23, IL-1β, TNF is impaired in human M1 macrophages with levels of IRF5 expression reduced by siRNA knock-down or in the peritoneal macrophages of Irf5−/− mice. The inventors provide the first insights into the molecular mechanisms behind IRF5's direct transcriptional activation of IL-12p40, IL-12p35 and IL-23p19 genes. Consequently, these macrophages set up the environment for a potent Th1/Th17 response. The inventors have also identified a new function of IRF5 as a transcriptional inhibitor of IL-10 and other selected M2-specific molecules. Global gene expression analysis demonstrates that exogenous IRF5 up- or down-regulates expression of established human markers of M1 or M2 (IL-12^{low}, IL-23^{low}, IL-10^{high}) macrophages respectively. Taken together, these data establish a new paradigm for macrophage polarisation in which IRF5 plays a critical role in M1 macrophage polarisation, and highlight the potential for therapeutic interventions via modulation of IRF5, and the IRF5-IRF4 balance.

In Example 2, the inventors have also shown that IRF5 induces secretion of TNF by human dendritic cells (DCs), which is essential for robust T cell activation by DCs. Through systematic bioinformatic and biochemical analyses of the TNF gene locus, the inventors have mapped two sites of IRF5 recruitment: 5′ upstream and 3′ downstream of the TNF gene. Remarkably, while IRF5 can directly bind to DNA in the upstream region, its recruitment to the downstream region depends on the protein-protein interactions with NF-κB RelA. In Example 3, the inventors have shown that IRF5 interacts with RelA via its IRF Association Domain (IAD), and in Example 4 the inventors have shown that IRF5 interacts with TRIM28. These Examples provide new insights into the molecular mechanisms employed by IRF5 to regulate gene expression and identifies IRF5-RelA and IRF5-TRIM28 interactions as a target for modulation of IRF5 activity.

Accordingly, a first aspect of the invention provides a method of treating a patient having an autoimmune disease, or a Th1 polarising infection, or a condition associated with inflammation other than asthma or allergy, the method comprising administering to the patient a therapeutically effective amount of an inhibitor of Interferon Regulatory Factor 5 (IRF5).

This aspect of the invention also provides an inhibitor of IRF5 for use in treating a patient having an autoimmune disease, or a Th1 polarising infection, or a condition associated with inflammation other than asthma or allergy. It further provides the use of an inhibitor of IRF5 in the preparation of a medicament for treating a patient having an autoimmune disease, or a Th1 polarising infection, or a condition associated with inflammation other than asthma or allergy.

In an embodiment, the autoimmune disease for treatment may be Crohn's disease, systemic lupus erythematosus (SLE), psoriasis, rheumatoid arthritis (RA), multiple sclerosis (MS), Sjogren's syndrome, inflammatory bowel disease (IBD) or atherosclerosis. The autoimmune disease for treatment may also be primary myxoedema, thyrotoxicosis, pernicious anaemia, autoimmune atrophic gastris, Addison's disease, insulin dependent diabetes mellitus (IDDM), Goodpasture's syndrome, myasthenia gravis, sympathetic ophthalmia, autoimmune haemolytic anaemia, idiopathic leucopenia, ulcerative colitis, dermatomyositis, scleroderma, mixed connective tissue disease, Hashimoto's disease, thyroiditis, Behcet's disease, coeliac disease/dermatitis herpetiformis, and demyelinating disease.

In an embodiment, the condition associated with inflammation other than asthma or allergy may be a condition associated with chronic inflammation, such as transplant rejection. In another embodiment, the condition associated with inflammation other than asthma or allergy may be a condition associated with acute inflammation, such as a response to injury or an ulcer.

In an embodiment, the Th1 polarising infection is a bacterial infection, such as infection with Escherichia coli, Legionella pneumophila, Listeria monocytogenes, Salmonella typhi, Mycobacterium tuberculosis, Mycobacterium ucerans, or a Streptococcus, or a viral infection such as an influenza virus, Sendai virus or Newcastle virus.

Other Th1 polarising bacterial and viral infections that are suitable for treatment by the methods of this aspect of the invention are listed in Tables 1 and 2. Thus, the invention can be considered to be a method for the treatment of these bacterial and viral infections by administration of a therapeutically effective amount of an IRF5 inhibitor. The invention may also be considered as a method for the treatment of a viral infection by administration of a therapeutically effective amount of an IRF5 inhibitor.

TABLE 1
Some common causes of disease in humans

| | | | |
|---|---|---|---|
| Viruses | DNA viruses | Adenoviruses | Human adenoviruses |
| | | | (e.g. types 3, 4, and 7) |
| | | Herpesviruses | Herpes simplex, varicella |
| | | | zoster, Epstein-Barr virus, |
| | | | cytomegalovirus |
| | | Poxviruses | Vaccinia virus |
| | | Parvoviruses | Human parvovirus |
| | | Papovaviruses | Papilloma virus |
| | | Hepadnaviruses | Hepatitis B virus |
| | RNA viruses | Orthomyxo- | Influenza virus |
| | | viruses | |
| | | Paramyxo- | Mumps, measles, respira- |
| | | viruses | tory syncytial virus |
| | | Coronaviruses | Common cold viruses |
| | | Picornaviruses | Polio, coxsackie, hepatitis |
| | | | A, rhinovirus |
| | | Reoviruses | Rotavirus, reovirus |
| | | Togaviruses | Rubella, arthropod-borne |
| | | | encephalitis |
| | | Flaviviruses | Arthropod-borne viruses, |
| | | | (yellow fever, dengue |
| | | | fever) |
| | | Arenaviruses | Lymphocytic chorio- |
| | | | meningitis, Lassa fever |
| | | Rhabdoviruses | Rabies |
| | | Retroviruses | Human T-cell leukemia |
| | | | virus, HIV |
| Bacteria | Gram +ve cocci | Staphylococci | Staphylococcus aureus |
| | | Streptococci | Streptococcus pneumoniae, |
| | | | S. pyogenes |
| | Gram +ve bacilli | | Corynebacteria, Bacillus |
| | | | anthracis, Listeria |
| | | | monocytogenes |
| | Gram −ve bacilli | | Salmonella, Shigella, |
| | | | Campylobacter, Vibrio, |
| | | | Yersinia, Pasteurella, |
| | | | Pseudomonas, |
| | | | Brucella, Haemophilus, |
| | | | Legionella, Bordetella |
| | Anaerobic | Clostridia | Clostridium tetani, |
| | bacteria | | C. botulinum, |
| | | | C. perfringens |
| | Spirochetes | | Treponema pallidum, |
| | | | Borrelia burgdorferi, |
| | | | Leptospira interrogans |
| | Mycobacteria | | Mycobacterium |
| | | | tuberculosis, |
| | | | M. leprae, M. avium |
| | Rickettsias | | Rickettsia prowazeki |
| | Chlamydias | | Chlamydia trachomatis |
| | Mycoplasmas | | Mycoplasma pneumoniae |

TABLE 2

| | Infectious agent | Disease |
|---|---|---|
| Viruses | Variola | Smallpox |
| | Varicella zoster | Chickenpox |
| | Epstein-Barr virus | Mononucleosis |
| | Influenza virus | Influenza |
| | Mumps virus | Mumps |
| | Measles virus | Measles |
| | Polio virus | Poliomyelitis |
| | Human immunodeficiency virus | AIDS |
| Bacteria | Staphylococcus aureus | Boils |
| | Streptococcus pyogenes | Tonsilitis |
| | Streptococcus pneumoniae | Pneumonia |
| | Corynebacterium diphtheriae | Diphtheria |
| | Clostridium tetani | Tetanus |
| | Treponema pallidum | Syphilis |
| | Borrelia burgdorferi | Lyme disease |
| | Salmonella typhi | Typhoid |
| | Vibrio cholerae | Cholera |
| | Legionella pneumophilia | Legionnaire's disease |
| | Rickettsia prowazeki | Typhus |
| | Chlamydia trachomatis | Trachoma |
| | Mycobacteria | Tuberculosis, leprosy |

It is appreciated that in certain circumstances, infections with Vibrio e.g., Vibrio cholerae, Mycobacteria, e.g., Mycobacterium leprae and Mycoplasma, e.g., Mycoplasma pneumoniae can be Th1 or Th2 polarising infections (and this is differs between their chronic and acute phases). Thus, in an embodiment, this aspect includes treating one of these bacterial infections with an IRF5 inhibitor during their Th1 polarising phases.

In a further specific embodiment, this aspect of the invention may not include treating a bacterial infection selected from Corynebacterium diphtheriae, Bacillus anthracia, Yersinia, Pasteurella, C. botulinum, C. perfringens or Rickettsia prowazeki with an IRF5 inhibitor.

By treating a condition we mean that the method can be used to alleviate symptoms of the disorder (i.e., the method is used palliatively), or to treat the disorder (i.e., the method is used to counter the underlying physiological basis for the disorder), possibly in combination with other suitable treatment agents.

The use of an inhibitor of IRF5 is believed to combat an undesirable autoimmune response directly, as well as treating its symptoms by directing T cells away from a pro-inflammatory role. Thus, treatment with an IRF5 inhibitor can be used as soon as the first symptoms of, e.g., an autoimmune disease, appear. Similarly, unlike other forms of treatment of certain autoimmune diseases, the method may be helpful in preventing inflammatory responses before they start. Thus, the method may be useful in treating patients who, for example because of their age or genetic factors, are strongly predisposed to an autoimmune disease before inflammatory symptoms show.

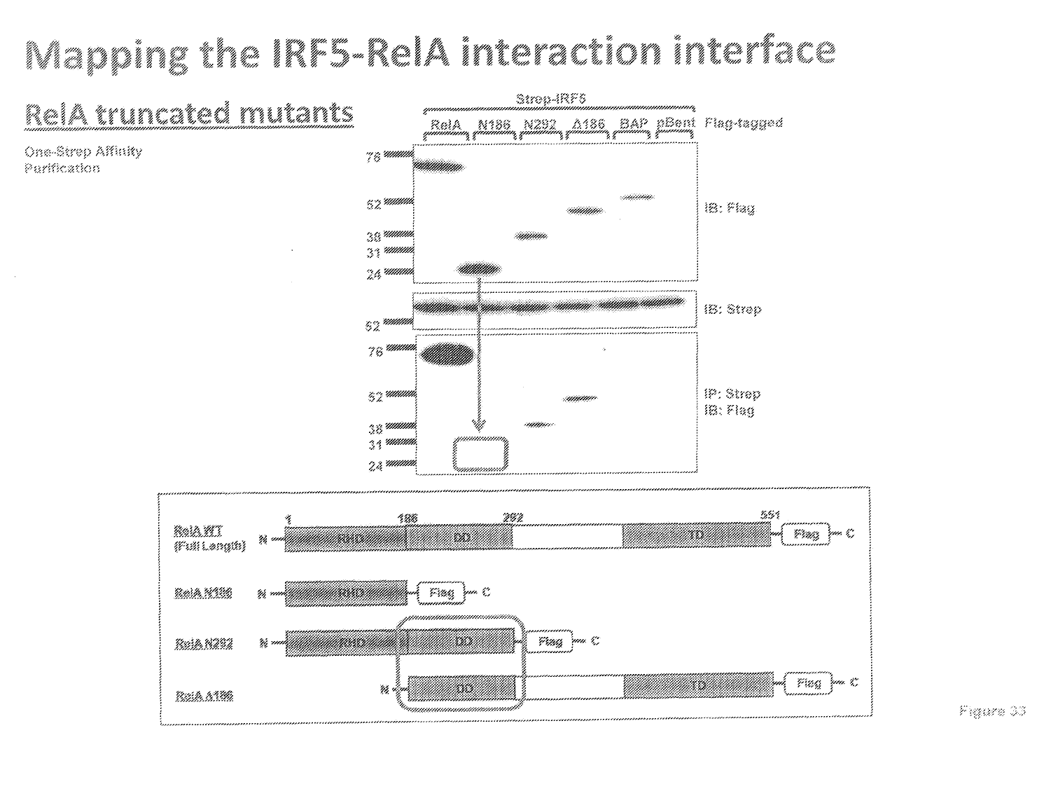

By Interferon Regulatory Factor 5 (IRF5), we include human IRF5, which is encoded by the human IRF5 gene located at chromosome 7q32 (OMIM ID 607218). IRF5 is a member of the IRF family; it is a transcription factor that possesses a helix-turn-helix DNA-binding motif and mediates virus- and interferon (IFN)-induced signalling pathways. It is appreciated that several isoforms/transcriptional variants of IRF5 are known and shown in Table 3. Of these, human IRF5 variant IRF5v3/4, also known as IRF5-203, which is encoded by transcriptional isoforms 3 and 4, is the most common isoform. The amino acid sequence of human IRF5v3/4 (SEQ ID No: 1) is provided in FIG. 34A and the cDNA sequence of isoforms 3 and 4 (SEQ ID No: 2) is provided in FIG. 34B.

TABLE 3

| Name | Ensembl Transcript ID | Length (bp) | Protein ID | Length (aa) | Biotype | CCDS |
|---|---|---|---|---|---|---|
| IRF5-001 | ENST00000402030 | 2786 | ENSP00000385352 | 498 | Protein coding | CCDS5808 |
| IRF5-002 | ENST00000357234 | 1680 | ENSP00000349770 | 514 | Protein coding | CCDS43645 |
| IRF5-003 | ENST00000489702 | 689 | ENSP00000418037 | 152 | Protein coding | — |
| IRF5-005 | ENST00000477535 | 1374 | ENSP00000419950 | 412 | Protein coding | — |
| IRF5-008 | ENST00000479582 | 637 | ENSP00000417770 | 141 | Protein coding | — |
| IRF5-009 | ENST00000473745 | 2282 | ENSP00000419149 | 498 | Protein coding | CCDS5808 |
| IRF5-010 | ENST00000464557 | 553 | ENSP00000419056 | 106 | Protein coding | — |
| IRF5-011 | ENST00000467002 | 560 | ENSP00000417454 | 59 | Protein coding | — |
| IRF5-201 | ENST00000249375 | 2749 | ENSP00000249375 | 498 | Protein coding | CCDS5808 |
| IRF5-202 | ENST00000342930 | 2851 | ENSP00000340338 | 505 | Protein coding | — |
| IRF5-203 | ENST00000412326 | 2848 | ENSP00000391780 | 488 | Protein coding | — |
| IRF5-204 | ENST00000419641 | 2803 | ENSP00000407935 | 489 | Protein coding | — |
| IRF5-205 | ENST00000430204 | 2796 | ENSP00000409106 | 227 | Protein coding | — |
| IRF5-206 | ENST00000453794 | 634 | ENSP00000414903 | 170 | Protein coding | — |
| IRF5-007 | ENST00000465603 | 2088 | ENSP00000418534 | 147 | Nonsense mediated decay | — |
| IRF5-012 | ENST00000473787 | 587 | ENSP00000420274 | 104 | Nonsense mediated decay | — |
| IRF5-004 | ENST00000488569 | 923 | No protein product | — | Retained intron | — |
| IRF5-006 | ENST00000461416 | 1057 | No protein product | — | Retained intron | — |

Preferably, the inhibitor of IRF5 inhibits at least one function or activity of human IRF5 variant IRF5v3/4 (referred to as IRF5-203, in Table 3). Thus, by an inhibitor of IRF5, we mean an agent that inhibits at least one function or activity of human IRF5 variant IRF5v3/4. However, it is appreciated that in some embodiments, the inhibitor of IRF5 may also inhibit at least one function or activity of another human IRF5 variant.

It is also well known that IRF5 is polymorphic, and a large number of polymorphisms, including SNPs are known. Thus, in an embodiment, the inhibitor of IRF5 also inhibits at least one function or activity of naturally-occurring variants of human IRF5v3/4 in which one or more of the amino acid residues have been replaced with another amino acid.

It is also appreciated that the IRF5 inhibitor may be one that inhibits at least one function or activity of an orthologue of IRF5 in another species, for example IRF5 from a horse, dog, pig, cow, sheep, rat, mouse, guinea pig or a primate. It will be appreciated, that when the inhibitor is administered to a particular individual, the inhibitor is one that modulates at least one function or activity of IRF5 from the same species as that individual. Thus, when the patient is a human patient, the inhibitor inhibits at least one function or activity of human IRF5, and so on.

Preferably, the patient is a human individual. However, when the patient is other than a human patient, it may be a non-human mammalian individual, such as a horse, dog, pig, cow, sheep, rat, mouse, guinea pig or primate. It is appreciated that the non-human patient may be an animal model of a human autoimmune disease or a Th1 polarising infection or a condition associated with inflammation other than asthma or allergy.

By an inhibitor of IRF5 we mean an agent that affects, e.g. inhibits, reduces or eliminates completely, any one or more functions or activities of the IRF5 protein. For example, the inhibitor may:
inhibit the binding of IRF5 to an IRF5 binding site in DNA;
interfere with or inhibit the binding of IRF5 to any of RelA, MyD88, TRAF6 or TRIM28;
inhibit the IRF5-mediated expression and/or secretion of TNF, IL-12, IL-23 and/or IL-1b from DCs and/or M1 macrophages;
inhibit or reverse the IRF5-mediated inhibition of expression and/or secretion of IL-10 from cells of the macrophage/monocyte lineage (e.g., it may induce expression and/or secretion of IL-10);
inhibit the IRF5-mediated upregulation of expression of one or more genes selected from the group consisting of CXCR3, CXCR4, CXCR5, CXCR7, EBI3, TNFSF4, TNFSF9, LTA, LTB, IFN-gamma, CCL1, CCL3, CXCL5, IL-19 and IL-32 in cells of the macrophage/monocyte lineage;
inhibit the IRF5-mediated downregulation of expression of one or more genes selected from the group consisting of CSF1R, IL-1R2, IL1RA and TGFβ in cells of the macrophage/monocyte lineage; or
inhibit or reverse the IRF5-mediated polarisation of cells of the macrophage/monocyte lineage towards the macrophage M1 phenotype (e.g., it may induce the polarisation of cells of the macrophage/monocyte lineage towards the macrophage M2 phenotype).

Preferably, the inhibitor of IRF5 has been shown to inhibit two or more of these functions or activities of IRF5, for example, three, four, five or more, or all, of these functions or activities of IRF5.

Suitable methods for determining inhibition of each of these functions or activities of IRF5 are known in the art and are described in the Examples.

By “cells of the macrophage/monocyte lineage” we include cells that are derived from monocyte precursors and include macrophages and monocytes. We also include monoctye-derived dendritic cells in humans, and bone marrow derived dendritic cells in the mouse.

Typically, the inhibitor of IRF5 may be a polynucleotide inhibitor of IRF5, a competitive inhibitor of IRF5, an agent that inhibits the expression of IRF5 in cells of the macrophage/monocyte lineage, a molecule that interferes with the IRF5-RelA interaction, or a dominant-negative mutant of IRF5.

In a preferred embodiment, the inhibitor of IRF5 may be a polynucleotide inhibitor of IRF5, which typically act to inhibit IRF5 expression. Various methodologies are known in the art for inhibiting IRF5 expression which can be applied in the context of the present invention. Suitable inhibitors of IRF5 expression include IRF5-specific RNAi, IRF5-specific short hairpin RNA (shRNA), IRF5-specific antisense (e.g., IRF5-specific morpholinos) and triplet-forming oligonucleotides, and IRF5-specific ribozymes. Thus in an embodiment, the polynucleotide inhibitor of IRF5 agent may be any of an antisense oligonucleotide, such as a morpholino, a short hairpin RNA (shRNA), a micro RNA (miRNA), a small interfering RNA (siRNA) or a ribozyme.

As is now well known in the art, suitable morpholinos, siRNA, shRNA, antisense or ribozyme agents can be made based on the knowledge of the IRF5 gene or cDNA sequence. Particular examples of suitable IRF5 siRNA sequences that may be used are provided in the Examples. IRF5 siRNA are commercially available, for example, as On-target SMMRT pool reagents from Dharmacon, USA (catalogue No. L-011706-00-0005), and from Santa Cruz Biotechnology, USA (catalogue No. sc-72044).

RNAi is the process of sequence-specific post-transcriptional gene silencing in animals initiated by double stranded RNA (dsRNA) that is homologous in sequence to the silenced gene (siRNA; Hannon et al (2002) Nature 418 (6894): 244-51; Brummelkamp et al (2002) Science 21, 21; and Sui et al (2002) Proc. Natl. Acad. Sci. USA 99, 5515-5520). The mediators of sequence-specific mRNA degradation are typically 21- and 22-nucleotide small interfering RNAs (siRNAs) which, in vivo, may be generated by ribonuclease III cleavage from longer dsRNAs. Duplex siRNA molecules selective for CG can readily be designed by reference to the amino acid sequences listed above. Typically, the first 21-mer sequence that begins with an AA dinucleotide which is at least 120 nucleotides downstream from the initiator methionine codon is selected. The RNA sequence perfectly complementary to this becomes the first RNA oligonucleotide. The second RNA sequence should be perfectly complementary to the first 19 residues of the first, with an additional UU dinucleotide at its 3′ end. Once designed based upon knowledge of the IRF5 cDNA sequence, the synthetic RNA molecules can be synthesised using methods well known in the art.

siRNAs may be introduced into cells in the patient using any suitable method, such as those described herein. Typically, the RNA is protected from the extracellular environment, for example by being contained within a suitable carrier or vehicle. Liposome-mediated transfer, e.g. the oligofectamine method, may be used.

Antisense oligonucleotides are single-stranded nucleic acids, which can specifically bind to a complementary nucleic acid sequence. By binding to the appropriate target sequence, an RNA-RNA, a DNA-DNA, or RNA-DNA duplex is formed. By binding to the target nucleic acid, antisense oligonucleotides can inhibit the function of the target nucleic acid. This may be a result of blocking the transcription, processing, poly(A) addition, replication, translation, or promoting inhibitory mechanisms of the cells, such as promoting RNA degradation. Typically, antisense oligonucleotides are 15 to 35 bases in length (Witters et al (1999) Breast Cancer Res Treat 53: 41-50 and Frankel et al (1999) J Neurosurg 91: 261-7). However, it is appreciated that it may be desirable to use oligonucleotides with lengths outside this range, for example 10, 11, 12, 13, or 14 bases, or 36, 37, 38, 39 or 40 bases. Thus, with knowledge of the IRF5 cDNA sequence, polynucleotide inhibitors of IRF5 expression can be produced using methods well known in the art.

The antisense molecules may be expressed from any suitable genetic construct and delivered to the patient. Typically, the genetic construct which expresses the antisense molecule comprises at least a portion of the IRF5 cDNA or gene operatively linked to a promoter which can express the antisense molecule in the cell. Preferably, the genetic construct is adapted for delivery to a human cell.

Ribozymes are RNA molecules capable of cleaving targeted RNA or DNA. Examples of ribozymes are described in, for example, U.S. Pat. No. 5,180,818; U.S. Pat. No. 5,168,053; U.S. Pat. No. 5,149,796; U.S. Pat. No. 5,116,742; U.S. Pat. No. 5,093,246; and U.S. Pat. No. 4,987,071, all incorporated herein by reference. Ribozymes specific for IRF5 can be designed by reference to the IRF5v3/4 cDNA sequence defined above using techniques well known in the art.

shRNA molecules may be sourced from the Sigma Aldrich Mission Library. However, shRNA molecules may be designed based upon knowledge of the IRF5 sequence for example by using a program called Oligoengine that identifies regions of an input sequence (i.e., IRF5) against which suitable oligonucleotides can be made.

Methods and routes of administering polynucleotide inhibitors, such as siRNA molecules, antisense molecules and ribozymes, to a patient, are well known in the art and described in more detail below.

It is appreciated that polynucleotide inhibitors of IRF5 may be administered directly, or may be administered in the form of a polynucleotide that encodes the inhibitor. Thus, as used herein, unless the context demands otherwise, by administering to the individual an inhibitor of IRF5 which is a polynucleotide, we include the meanings of administering the inhibitor directly, or administering a polynucleotide that encodes the inhibitor, typically in the form of a vector.

The terms “nucleic acid molecule” and “polynucleotide” may be used interchangeably, and refer to a polymer of nucleotides. Such polymers of nucleotides may contain natural and/or non-natural nucleotides, and include, but are not limited to, DNA, RNA, and PNA.

The terms “polypeptide” and “protein” are used interchangeably, and refer to a polymer of amino acid residues. Except when the context requires otherwise, such polymers of amino acid residues may contain natural and/or non-natural amino acid residues. The terms “polypeptide” and “protein” also include post-translationally modified polypeptides and proteins, including, for example, glycosylated, sialylated, acetylated, and/or phosphorylated polypeptides and proteins.

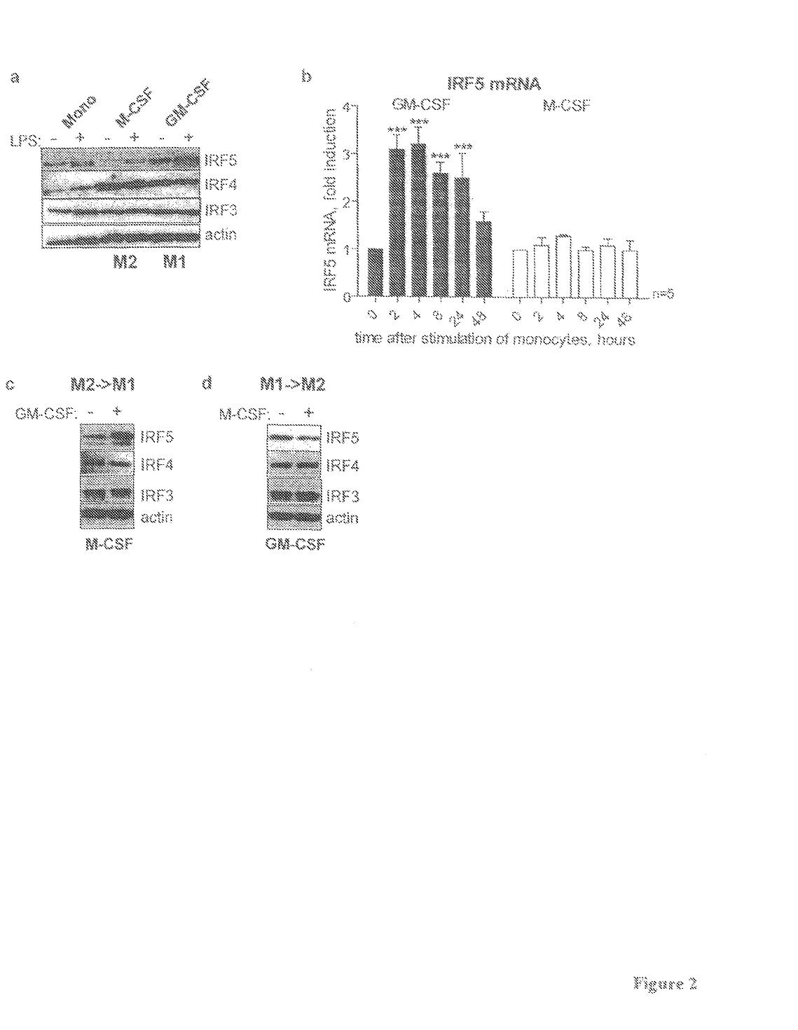

In another embodiment, the agent that inhibits the expression of IRF5 in cells of the macrophage/monocyte lineage may be macrophage-colony stimulating factor (M-CSF) or an M-CSF receptor agonist. As shown in FIG. 2c, M1 macrophages were treated with M-CSF and the level of IRF5 protein reduced.

In another embodiment, the competitive inhibitor of IRF5 is IRF4, which has been shown to promote M2 macrophage differentiation (Satoh, T. et al. (2010) Nat Immunol 11, 936-944), to negatively regulate Tol-like receptor signalling by competing with IRF5 for MyD88 interactions (Negishi, H. et al. (2005) Proc Natl Acad Sci USA 102, 15989-15994), and to target IRF5 to regulate Epstein-Barr virus transformation (Xu, D et al (2011) J. Biol. Chem. 286(20): 18261-267).

In an alternative embodiment, the inhibitor of IRF5 is not IRF4.

In a further embodiment, the inhibitor may be a molecule that interferes with the IRF5-RelA interaction, such as an IRF5 molecule that has a mutated or deleted IAD domain, which is located at residues 219-395 of IRF5v3/4. Such an inhibitor may also be considered to be a dominant-negative mutant of IRF5.

Thus, in a further embodiment, the inhibitor may be a dominant-negative mutant of IRF5. As well as those mentioned above, the dominant-negative mutant may have a mutated or deleted DNA binding domain (DBD), for example as described below in Example 2. The DBD of IRF5v3/4 is at amino acid residues 1-136. Specific examples of mutations that have dominant-negative effect include a mutation at Alanine at position 68, especially when substituted with Proline, which results in complete loss of DNA binding activity (Yang et al (2009), Plos One v4(5):e5500), see Example 2.

Suitable methods, routes and compositions for preparing polypeptide inhibitors of IRF5 and nucleic acid molecules that encode them and administering them to a patient are known in the art and described below, and include viral vectors such as adenoviral vectors.

Further inhibitors of IRF5 can be identified using the screening methods described below.

A second aspect of the invention provides a method of treating a patient having a condition selected from a compromised immune system and a Th2 polarising infection, the method comprising administering to the patient a therapeutically effective amount of IRF5, or an agonist of IRF5, or an agent that induces the expression of IRF5 in cells of the macrophage/monocyte lineage.

Typically, the patient having a compromised immune system may be a patient with too many infections, opportunistic infections with a non-pathogen, a low T cell count, and/or low Ig levels, often has allergy, and may have symptoms similar to AIDS at a late/severe stage in need of treatment.

In an embodiment, the Th2 polarising infection is a parasitic infection, such as an infection with a helminth, a flatworm, a roundworm of the genus Ascaris, Leishmania major or Trypanosoma brucei, or a bacterial infection with Neisseria meningitides or Neisseria gonorrhoeae, or a fungal infection such as Candida, or a Cryptococcus.

Infections whose treatment may be aided using the method of the second aspect of the invention include fungal infections such as Candida albicans, Cryptococcus neoformans, Aspergillus, Histoplasma capsulatum, Coccidioides immitis, Pneumocystis carinii; infections by protozoa such as Entamoeba histolytica, Giardia, Leishmania, Plasmodium, Trypanosoma, Toxoplasma gondii, and Cryptosporidium; and infections by worms such as Trichuris trichura, Trichinella spiralis, Enterobius vermicularis, Ascaris lumbricoides, Ancylostoma, Strongyloides, Filaria, Onchocerca volvulus, Loa boa, Dracuncula medinensis; Schistosoma, and Clonorchis sinensis.

Thus, this aspect of the invention may be considered to be a method of treating or aiding in the treatment of a fungal, protozoal, parasitic or worm infection, such as an infection with the organisms mentioned above, the method comprising administering to the patient a therapeutically effective amount of IRF5, or an agonist of IRF5, or an agent that induces the expression of IRF5 in cells of the macrophage/monocyte lineage.

In another embodiment, the Th2 polarising infection may be a persistent mycobacterial infection e.g., persistent Mycobacterium leprae infection, which is often due to an insufficient Th1 response. Thus, boosting the Th1 response in a patient, using the method of the second aspect of the invention, would be expected to be helpful in treating persistent mycobacterial infection. Similarly, when an infection with Vibrio e.g., Vibrio cholerae or a Mycoplasma, e.g., Mycoplasma pneumoniae is in a Th2 polarising phase, using the method of the second aspect of the invention to boost the Th1 response would be expected to be helpful.

In the context of administering IRF5 to a patient we include the meaning of administering IFR5 polypeptide, or a variant thereof having at least 90% sequence identity with the IRF5 polypeptide, or a nucleic acid molecule which encodes the IRF5 polypeptide or variant thereof.

Preferably, the IRF5 to be administered to a patient is human IRF5v3/4, and a variant thereof is a variant of human IRF5v3/4. However, in other embodiments, other IRF5 isoforms or variants thereof having at least 90% sequence identity with that isoform, may be administered to a patient.

In an embodiment, the variant of the IFR5 polypeptide has at least 91% sequence identity, or at least 92% sequence identity, or at least 93% sequence identity, or at least 94% sequence identity, or at least 95% sequence identity, or at least 96% sequence identity, or at least 97% sequence identity, or at least 98% sequence identity, or at least 99% sequence identity, with the sequence of the IFR5 polypeptide of which it is a variant. Thus, preferably, the variant of the IFR5 polypeptide has at least 91%, 92%, 93%, 94%, 95%, 96%, 97%, 98% or 99% sequence identity with the sequence of the IFR5v3/4 polypeptide. Such variants may be made, for example, using the methods of recombinant DNA technology, protein engineering and site-directed mutagenesis, which are well known in the art, and discussed in more detail below.

The percent sequence identity between two polypeptides may be determined using suitable computer programs, for example the GAP program of the University of Wisconsin Genetic Computing Group and it will be appreciated that percent identity is calculated in relation to polypeptides whose sequence has been aligned optimally. The alignment may alternatively be carried out using the Clustal W program (Thompson et al., (1994) Nucleic Acids Res 22, 4673-80). The parameters used may be as follows: Fast pairwise alignment parameters: K-tuple (word) size; 1, window size; 5, gap penalty; 3, number of top diagonals; 5. Scoring method: x percent. Multiple alignment parameters: gap open penalty; 10, gap extension penalty; 0.05. Scoring matrix: BLOSUM.

It is preferred that the variant of the IFR5 possesses at least 50% of the activity of full length human IFR5v3/4 in polarising cells of the macrophage/monocyte lineage towards the macrophage M1 phenotype. It is more preferred if the variant of IFR5 possesses at least 60%, or at least 70%, or at least 80%, or at least 90%, or at least 95%, or at least 99%, or 100% or more, of the activity of full length human IFR5v3/4 in polarising cells of the macrophage/monocyte lineage towards the macrophage M1 phenotype. This can be determined using the methods described in Example 1.

Additionally or alternatively, IRF5 activity may be measured by the ability (e.g., of the variant) to inhibit expression and/or secretion of IL-10 from cells of the macrophage/monocyte lineage; upregulate expression of one or more genes selected from the group consisting of CXCR3, CXCR4, CXCR5, CXCR7, EBI3, TNFSF4, TNFSF9, LTA, LTB, IFN-gamma, CCL1, CCL3, CXCL5, IL-19 and IL-32 in cells of the macrophage/monocyte lineage; or downregulate expression of one or more genes selected from the group consisting of CSF1R, IL-1R2, IL1RA and TGFβ in cells of the macrophage/monocyte lineage (as described in Example 1), or to induce expression and/or secretion of TNF from DCs (as described in Example 2).

Typically, and suitably, the nucleic acid molecule which encodes the IRF5 polypeptide or variant thereof is administered to the patient via a gene therapy vector, such as a viral vector that encodes the polypeptide. Many suitable vectors are known in the art and are described for example in U.S. Pat. Nos. 7,323,450 and 6,730,512 and US Patent Application No. 2010/0203027 which describe gene therapy using vectors that encode a cytokine.

In an embodiment, the agent that induces the expression of IRF5 in cells of the macrophage/monocyte lineage may be granulocyte macrophage-colony stimulating factor (GM-CSF), a GM-CSF receptor agonist, IFN-gamma, IL-23 (Li et al, ACR/ARHP 2011 Scientific Meeting, Presentation 2097), and STAT1 inducing molecules such as IFNα, β, or λ, or a cytokine or growth factor such as IL-27, EGF, or CSF.

By “GMCSF” we include the gene product of the human GMCSF gene and naturally occurring variants thereof. The nucleotide and the amino acid sequence of human GMCSF is found in GenBank Accession No. NM_{—}000758. Some naturally occurring variants of GMCSF are also listed in NM_{—}000758. GMCSF is also known as colony stimulating factor 2 (CSF2). GMCSF and analogues thereof are described in U.S. Pat. Nos. 5,229,496; 5,391,485; U.S. Pat. Nos. 5,393,870; and 5,602,007.

While it is preferred that GMCSF is human GMCSF as defined above, by GMCSF we also include GMCSF from other species. It is appreciated that for applications in which GMCSF is administered to a non-human subject, the GMCSF is preferably from the same species as the subject. If the GMCSF is administered to a human subject, the GMCSF is preferably human GMCSF or a derivative thereof.

A preferred GMCSF for the practice of this invention is sargramostim, the proper name for yeast-derived recombinant human GMCSF, sold under the trade name Leukine® produced by Immunex, Inc. Leukine® is a recombinant human GMCSF produced in a S. cerevisiae expression system. Leukine® is a glycoprotein of 127 amino acids characterised by 3 primary molecular species having molecular masses of 19,500, 16,800 and 15,500 Daltons. The amino acid sequence of Leukine® differs from natural human GMCSF by a substitution of leucine at position 23, and the carbohydrate moiety may be different from the native protein. Leukine® is suitable for subcutaneous or intravenous administration (Leukine® Package Insert Approved Text, February 1998).

Another GMCSF suitable for the practice of this invention is molgramostim, the proper name for E. coli-derived recombinant human GMCSF, sold under the trade name Leucomax® (Schering-Plough). Leukomax® is a recombinant human GMCSF produced in an E. coli expression system. Leucomax® is a water soluble, non-glycosylated protein of 127 amino acids having a molecular mass of 14,477 Daltons. The amino acid sequence of Leucomax® differs from natural human GMCSF by a substitution of isoleucine at position 100. Leucomax® is available as a powder which, once reconstituted, is suitable for subcutaneous or intravenous administration (Leucomax® Data Sheet, November 2002).

A further GMCSF suitable for the practice of this invention is regramostim, the proper name for CHO-derived recombinant human GMCSF. Regramostim is a recombinant human GMCSF of 127 amino acids that is more highly glycosylated than sargramostim.

A third aspect of the invention provides a method of treating a patient having cancer, the method comprising administering to the patient a therapeutically effective amount of IRF5, or an agonist thereof.

In an embodiment, the cancer may be a cancer, for example an epithelial cancer, such as cancer of the liver, breast, colon, lung, prostate, pancreas or skin, or may be lymphoma or leukaemia.

In the context of administering IRF5 to a patient we include the meaning of administering IFR5 polypeptide, or a variant thereof having at least 90% sequence identity with the IRF5 polypeptide, or a nucleic acid molecule which encodes the IRF5 polypeptide or variant thereof, as described above. Further preferences for the IRF5 and agonist thereof are as described above.

Polypeptides, such as the IRF5 or variant thereof, may be prepared using an in vivo or in vitro expression system. Preferably, an expression system is used that provides the polypeptides in a form that is suitable for pharmaceutical use, and such expression systems are known to the skilled person. As is clear to the skilled person, polypeptides of the invention suitable for pharmaceutical use can be prepared using techniques for peptide synthesis.

A nucleic acid molecule encoding, for example, the IRF5 or variant thereof, may be used to transform a host cell or host organism for expression of the desired polypeptide. Suitable hosts and host cells are known in the art and may be any suitable fungal, prokaryotic or eukaryotic cell or cell line or organism, for example: bacterial strains, including gram-negative strains such as Escherichia coli and gram-positive strains such as Bacillus subtilis or of Bacillus brevis; yeast cells, including Saccharomyces cerevisiae; or Schizosaccharomyces pombe; amphibian cells such as Xenopus oocytes; insect-derived cells, such SF9, Sf21, Schneider and Kc cells; plant cells, for example tobacco plants; or mammalian cells or cell lines, CHO-cells, BHK-cells (for example BHK-21 cells) and human cells or cell lines such as HeLa, COS (for example COS-7) and PER.C6® cells; as well as all other hosts or host cells that are known and can be used for the expression and production of polypeptides.

For production on an industrial scale, preferred heterologous hosts for the (industrial) production of polypeptides, such as IRF5 or variant thereof, include strains of E. coli and S. cerevisiae that are suitable for large scale expression/production/fermentation, and in particular for large scale pharmaceutical (i.e. GMP grade) expression/production/fermentation. Suitable examples are commercially available by companies such as Biovitrum (Uppsala, Sweden). Alternatively, mammalian cell lines, in particular CHO cells, can be used for large scale expression/production/fermentation, and in particular for large scale pharmaceutical expression/production/fermentation. Again, such expression/production systems are commercially available.

The choice of the specific expression system depends, in part, on the requirement for certain post-translational modifications, more specifically glycosylation. The production of a protein for which glycosylation is desired or required necessitates the use of mammalian expression hosts that have the ability to glycosylate the expressed protein. The glycosylation pattern obtained (i.e., the kind, number and position of residues attached) will depend on the cell or cell line that is used for the expression. Preferably, either a human cell or cell line is used (i.e., leading to a protein that essentially has a human glycosylation pattern) or another mammalian cell line is used that can provide a glycosylation pattern that is essentially and/or functionally the same as human glycosylation or at least mimics human glycosylation. Generally, prokaryotic hosts such as E. coli do not have the ability to glycosylate proteins, and the use of lower eukaryotes such as yeast usually leads to a glycosylation pattern that differs from human glycosylation. Nevertheless, it should be understood that all the described host cells and expression systems may be used in the invention, depending on the desired polypeptide amino acid sequence to be obtained and its desired use. Thus, according to one embodiment, the polypeptides, such as IRF5 or variant thereof, may be glycosylated. According to an alternative embodiment, it may not be glycosylated.

When expression in a host cell is used to produce the polypeptide, it can be produced either intracellullarly (e.g. in the cytosol, in the periplasm or in inclusion bodies) and then isolated from the host cells and optionally further purified; or they can be produced extracellularly (e.g. in the medium in which the host cells are cultured) and then isolated from the culture medium and optionally further purified. When eukaryotic host cells are used, extracellular production is usually preferred since this considerably facilitates further isolation and downstream processing as is well known in the art.

Many suitable promoters for expression of a desired polypeptide are known, as are many suitable vectors and many suitable secretory sequences. Suitable techniques for transforming a host cell with the nucleotide sequence encoding the desired polypeptide, and for detecting and selecting those cells that have been successfully transformed are also very well known in the art. Preferably, the host cells express, or are capable of expressing (e.g. under suitable conditions) the desired polypeptide. To produce/obtain expression of the polypeptide, the transformed host cell may generally be kept, maintained and/or cultured under conditions such that the polypeptide is expressed/produced. Suitable conditions are also well known to the skilled person and depend upon the host cell/host organism used, as well as on the regulatory elements that control the expression of the (relevant) nucleotide sequence. In addition, many suitable techniques for isolating and purifying the polypeptide once it has been expressed are known.

Less preferably, the polypeptide, such as IRF5 or variant thereof, may be made by chemical synthesis, again using methods well known in the art for many years.

Nucleic acid molecules, for examples polynucleotide inhibitors of IRF5, nucleic acid molecules encoding polynucleotide inhibitors of IRF5, and nucleic acid molecules encoding IRF5 or the variant thereof, may be prepared using methods very well known in the art of molecular biology. For example, many of the techniques used in connection with recombinant DNA, oligonucleotide synthesis, tissue culture and transformation (e.g., electroporation, lipofection), enzymatic reactions, and purification techniques are known in the art. Many such techniques and procedures are described, e.g., in Sambrook et al., Molecular Cloning: A Laboratory Manual (3^{rd} edition, Cold Spring Harbor Laboratory Press, Cold Spring Harbor, N.Y. (2001)), among other places.

In certain embodiments, polypeptides for administration to a patient, such as the IRF5 or variant thereof, may be in the form of a fusion molecule in which the polypeptide is attached to a fusion partner to form a fusion protein. Many different types of fusion partners are known in the art. One skilled in the art can select a suitable fusion partner according to the intended use of the fusion protein. Examples of fusion partners include polymers, polypeptides, lipophilic moieties, and succinyl groups. Certain useful protein fusion partners include serum albumin and an antibody Fc domain, and certain useful polymer fusion partners include, but are not limited to, polyethylene glycol, including polyethylene glycols having branched and/or linear chains. In certain embodiments, the polypeptide may be PEGylated, or may comprise a fusion protein with an Fc fragment.

In an embodiment, the polypeptide may be fused to or may comprise additional amino acids in a sequence that facilitates entry into cells (i.e. a cell-penetrating peptide). Thus, for example, the IRF5 or variant thereof or a polypeptide IRF5 inhibitor may further comprise the sequence of a cell-penetrating peptide (also known as a protein transduction domain) that facilitates entry into cells.

Thus, for example the IRF5 or variant thereof may further comprise the sequence of a cell-penetrating peptide (also known as a protein transduction domain) that facilitates entry into cells. As is well known in the art, cell-penetrating peptides are generally short peptides of up to 30 residues having a net positive charge and act in a receptor-independent and energy-independent manner (Lindgren et al (2000) Trends Pharmacol. Sci. 21(3): 99-103; Deshayes et al (2005) Curr. Pharm. Des. 11(28): 3629-38; Deshayes et al (2005) Cell Mol Life Sci. 62(16): 1839-49; Takeuchi et al (2006). ACS Chem. Biol. 1(5): 299-303), the entire disclosures of which relating to cell-penetrating peptides are incorporated herein by reference).

Among the best characterised cell-penetrating peptides is the Antennapedia-derived peptide (e.g., see Derosssi at al (1994) J. Biol. Chem. 269: 10444-50; Derosssi et al (1996) J. Biol. Chem. 271: 18188-93; and Prochiantz (1996) Curr. Opin. Neurobiol. 6: 629-634)), which is a 16-residue polypeptide (RQIKIWFQNRRMKWKK; SEQ ID No: 3) corresponding to residues 43-58 (i.e. the third helix of the homeodomain) of Antennapedia, a Drosophila transcription factor. The entire disclosure of Derosssi et al and Prochiantz relating to cell-penetrating peptides is incorporated herein by reference. An Antennapedia-derived peptide is commercially available as Penetratin™ (http://www.qbiogene.com/products/transfection/penetratin.shtml) from Quantum Biotechnologies. Penetratin™ is a 16-amino acid peptide corresponding to the third helix of the homeodomain of Drosophila Antennapedia (pAntp) protein. This peptide is able to translocate across biological membranes by an energy-independent mechanism. With the use of the Penetratin™ peptide, covalently attached peptides are internalised and conveyed to the cytoplasm and nucleus in a wide variety of cell types.

Console et al (2003) J. Biol. Chem. 278(37): 35109-14 described protein transcription domains derived from Antennapedia (SGRQIKIWFQNRRMKWKKC; SEQ ID No: 4) and HIV-1 TAT (SGYGRKKRRQRRRC; SEQ ID No: 5) that mediate the uptake of molecules such as polypeptides into cells. Suzuki at al (2002, J. Biol. Chem. 277(4): 2437-43) described arginine-rich proteins including HIV-1 Rev (34-50) and octoarginine that are efficiently translocated through the cell membrane and act as protein carriers. The sequence of these cell-penetrating peptides is listed in Table 1 of Suzuki et al (2002). Jones et al (2005, Br. J. Pharmacol. 145(8): 1093-1102) characterised the peptide-mediated delivery of four cell-penetrating peptides, including peptides derived from Antennapedia, TAT, Transportan and a polyarginine peptide. The sequence of these cell-penetrating peptides is listed in Table 1 of Jones et al (2005). Further cell-penetrating peptides include the S4₁₃-PV and Pep-1 peptides derived from dermaseptin S4 and the SV40 large T nuclear localisation sequence (Mano et al (2005) Biochem J. 390(Pt 2): 603-612). The sequence of these cell-penetrating peptides is listed in Table 1 of Mano et al (2005). De Coupade et al (2005, Biochem. J. 390: 407-418) describe ten cell-penetrating peptides of 14-22 residues in length that are able to transport other peptides to the cytoplasm or nucleus of target cells. These peptides, referred to as Vectocell® penetrating peptides, were derived from superoxide dismutase, platelet-derived growth factor, epidermal-like growth factor, intestinal mucin, CAP37, superoxide dismutase and intestinal mucin, intestinal mucin and PDGF, and apolipoprotein B and anti-DNA antibody. The sequence of the Vectocell® penetrating peptides is listed in Table 1 of de Coupade et al (2005). The entire disclosure of each of these publications that relates to cell-penetrating peptides is incorporated herein by reference.

Additionally or alternatively, the polypeptide may be fused to or may comprise additional amino acids in a sequence that facilitates entry into the nucleus (i.e., a nuclear localisation sequence (NLS), aka nuclear localisation domain (NLD)). Thus, for example, the IRF5 or variant thereof may further comprise the sequence of an NLS that facilitates entry into the nucleus. By NLS we include any polypeptide sequence that, when fused to a target polypeptide, is capable of targeting it to the nucleus. Typically, the NLS is one that is not under any external regulation (eg calcineurin regulation) but which permanently translocates a target polypeptide to the nucleus. Methods for determining whether a particular protein is capable of translocating to the nucleus are well known in the art and include, for example, immunohistological techniques. It is appreciated that, like cell-penetrating peptides, NLS sequences typically consist of one or more stretches of positively charged lysine or arginine residues and any such suitable sequence may be used, for example the NLS from SV40 large T antigen, nucleoplasmin, C-myc, the acidic M9 domain of hnRNP A1, the yeast transcription repressor Matα2, and from UsnRNPs as is well known in the art (see, for example, Kalderon et al (1984) Cell 39: 499-509; Dingwall et al (1988) J. Cell Biol. 107 (3): 841-9; Chelsky et al (1989) Mol. Cell Biol. 9(6): 2487-92; Makkerh et al (1996) Curr Biol. 6 (8): 1025-7; and Mattaj et al (1998) Annu Rev Biochem. 67: 265-306).

It is appreciated that the sequence of the cell-penetrating peptide and/or the NLS may be adjacent to the sequence of the IRF5 or variant or polypeptide inhibitor of IRF5, or these sequences may be separated by one or more amino acids residues, such as glycine residues, acting as a spacer as described in detail below.

It is also appreciated that the use of a viral vector, such as an adenoviral vector, including those discussed herein, also facilitates entry of therapeutic nucleic acid or protein into the cell nucleus.

Therapeutic proteins produced as an Fc-chimera are known in the art. For example, Etanercept, the extracellular domain of TNFR2 combined with an Fc fragment, is a therapeutic polypeptide used to treat autoimmune diseases, such as rheumatoid arthritis.

In certain embodiments, the fusion partner may be a polymer, for example, polyethylene glycol (PEG). PEG may comprise branched and/or linear chains. In certain embodiments, a fusion partner comprises a chemically-derivatised polypeptide having at least one PEG moiety attached.

The fusion partner may be attached, either covalently or non-covalently, to the amino-terminus or the carboxy-terminus of the polypeptide. The attachment may also occur at a location within the polypeptide other than the amino-terminus or the carboxy-terminus, for example, through an amino acid side chain (such as, for example, the side chain of cysteine, lysine, histidine, serine, or threonine).

In either covalent or non-covalent attachment embodiments, a linker may be included between the fusion partner and the polypeptide, such bas the IRF5 or variant thereof. Such linkers may be comprised of amino acids and/or chemical moieties. One skilled in the art can select a suitable linker depending on the attachment method used, the intended use of the polypeptide, and the desired spacing between the polypeptide and the fusion partner.

Exemplary methods of covalently attaching a fusion partner to a polypeptide include, but are not limited to, translation of the polypeptide and the fusion partner as a single amino acid sequence, and chemical attachment of the fusion partner to the polypeptide. When the fusion partner and the polypeptide are translated as single amino acid sequence, additional amino acids may be included between the fusion partner and the polypeptide as a linker. In certain embodiments, the linker is glycine-serine (“GS”). In certain embodiments, the linker is selected based on the polynucleotide sequence that encodes it, to facilitate cloning the fusion partner and the polypeptide into a single expression construct (for example, a polynucleotide containing a particular restriction site may be placed between the polynucleotide encoding the fusion partner and the polynucleotide encoding the polypeptide, wherein the polynucleotide containing the restriction site encodes a short amino acid linker sequence).

When the fusion partner and the polypeptide are covalently coupled by chemical means, linkers of various sizes can typically be included during the coupling reaction. One skilled in the art can select a suitable method of covalently attaching a fusion partner to a polypeptide depending, for example, on the identity of the fusion partner and the particular use intended for the fusion molecule. One skilled in the art can also select a suitable linker type and length, if one is desired.

Exemplary methods of non-covalently attaching a fusion partner to a polypeptide include, but are not limited to, attachment through a binding pair. Exemplary binding pairs include, but are not limited to, biotin and avidin or streptavidin, an antibody and its antigen, etc. Again, one skilled in the art can select a suitable method of non-covalently attaching a fusion partner to a polypeptide depending, for example, on the identity of the fusion partner and the particular use intended for the fusion molecule. The selected non-covalent attachment method should be suitable for the conditions under which the fusion molecule will be used, taking into account, for example, the pH, salt concentrations, and temperature.

It is appreciated that the polypeptide or nucleic acid molecule for administration to the patient may be formulated as a nanoparticle. Nanoparticles are a colloidal carrier system that has been shown to improve the efficacy of an encapsulated drug by prolonging the serum half-life. Polyalkylcyanoacrylates (PACAs) nanoparticles are a polymer colloidal drug delivery system that is in clinical development (described, for example, by Stella et al (2000) J. Pharm. Sci., 89: 1452-1464; Brigger et al (2001) Int. J. Pharm 214: 37-42; Calvo et al (2001) Pharm. Res. 18: 1157-1166; and Li et al (2001) Biol. Pharm. Bull. 24: 662-665). Biodegradable poly(hydroxyl acids), such as the copolymers of poly(lactic acid) (PLA) and poly(lactic-co-glycolide) (PLGA) are being extensively used in biomedical applications and have received FDA approval for certain clinical applications. In addition, PEG-PLGA nanoparticles have many desirable carrier features including (i) that the agent to be encapsulated comprises a reasonably high weight fraction (loading) of the total carrier system; (ii) that the amount of agent used in the first step of the encapsulation process is incorporated into the final carrier (entrapment efficiency) at a reasonably high level; (iii) that the carrier has the ability to be freeze-dried and reconstituted in solution without aggregation; (iv) that the carrier be biodegradable; (v) that the carrier system be of small size; and (vi) that the carrier enhances the particles persistence.

Nanoparticles may be synthesised using virtually any biodegradable shell known in the art. In one embodiment, a polymer, such as poly(lactic-acid) (PLA) or poly(lactic-co-glycolic acid) (PLGA) is used. Such polymers are biocompatible and biodegradable, and are subject to modifications that desirably increase the photochemical efficacy and circulation lifetime of the nanoparticle. In one embodiment, the polymer is modified with a terminal carboxylic acid group (COOH) that increases the negative charge of the particle and thus limits the interaction with negatively charged nucleic acids. Nanoparticles may also be modified with polyethylene glycol (PEG), which also increases the half-life and stability of the particles in circulation. Alternatively, the COOH group may be converted to an N-hydroxysuccinimide (NHS) ester for covalent conjugation to amine-modified compounds.

Other protein modifications to stabilise a polypeptide, for example to prevent degradation, as are well known in the art may also be employed. Specific amino acids may be modified to reduce cleavage of the polypeptide in vivo; typically, N- or C-terminal regions are modified to reduce protease activity on the polypeptide. A stabilising modification is any modification capable of stabilising a protein, enhancing the in vitro half life of a protein, enhancing circulatory half life of a protein and/or reducing proteolytic degradation of a protein. For example, polypeptides may be linked to the serum albumin or a derivative of albumin. Methods for linking polypeptides to albumin or albumin derivatives are well known in the art (e.g., U.S. Pat. No. 5,116,944).

It is appreciated that the compounds for administration to a patient, for example as described above, will normally be formulated as a pharmaceutical composition, i.e. together with a pharmaceutically acceptable carrier, diluent or excipient.

By “pharmaceutically acceptable” is included that the formulation is sterile and pyrogen free. Suitable pharmaceutical carriers, diluents and excipients are well known in the art of pharmacy. The carrier(s) must be “acceptable” in the sense of being compatible with the compound and not deleterious to the recipients thereof. Typically, the carriers will be water or saline which will be sterile and pyrogen free; however, other acceptable carriers may be used.

Since the treatment agents to be used in the above aspects of the invention, i.e., the IRF5, IRF5 inhibitors, IRF5 agonists and IRF5 inducers, act on cells of monocyte/macrophage lineage, it is preferred that they are administered systemically into the circulation where these cells are located. Thus, in a preferred embodiment, the pharmaceutical compositions or formulations for administration to a patient are formulated for parenteral administration, more particularly for intravenous administration. In a preferred embodiment, the pharmaceutical composition is suitable for intravenous administration to a patient, for example by injection.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

It is also appreciated that macrophages are already present in sites of inflammation. Thus, for the treatment of the autoimmune disease rheumatoid arthritis, the IRF5 inhibitor may be administered directly to an inflamed joint. Similarly, by way of another example, for the treatment of the autoimmune disease psoriasis, the IRF5 inhibitor may be administered directly to the skin.

Thus, in an alternative embodiment, the pharmaceutical composition is suitable for topical administration to a patient.

It is also appreciated that for the treatment of cancer, the IRF5 or agonist thereof or polynucleotide encoding the IRF5 may be administered directly to the site of the cancer, for example, injected directly into the cancer.

Preferably, the formulation is a unit dosage containing a daily dose or unit, daily sub-dose or an appropriate fraction thereof, of the active ingredient.

In human therapy, the compound will generally be administered in admixture with a suitable pharmaceutical excipient, diluent or carrier selected with regard to the intended route of administration and standard pharmaceutical practice.

The treatment agents can be administered parenterally, for example, intravenously, intra-arterially, intraperitoneally, intra-muscularly or subcutaneously, or directly into a joint, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

For parenteral administration to human patients, the daily dosage level of a compound can typically be from 1 to 1,000 mg per adult (i.e. from about 0.015 to 15 mg/kg), administered in single or divided doses. The physician will in any event determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

In certain embodiments, the compound may be applied topically in the form of a lotion, solution, cream, ointment or dusting powder, or may be transdermally administered, for example, by the use of a skin patch. For application topically to the skin, the compound can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

In certain embodiments, the compound can also be delivered by electroincorporation (EI). EI occurs when small particles of up to 30 microns in diameter on the surface of the skin experience electrical pulses identical or similar to those used in electroporation. In EI, these particles are driven through the stratum corneum and into deeper layers of the skin. The particles can be loaded or coated with the compound or can simply act as “bullets” that generate pores in the skin through which the compound can enter.

In an embodiment, when the compound is a polypeptide, it may be delivered using an injectable sustained-release drug delivery system. These are designed specifically to reduce the frequency of injections. An example of such a system is Nutropin Depot which encapsulates recombinant human growth hormone (rhGH) in biodegradable microspheres that, once injected, release rhGH slowly over a sustained period.

In an embodiment, the agents for treatment can be administered by a surgically implanted device that releases the drug directly to the required site, for example a site of inflammation in rheumatoid arthritis. Such direct application to the site of disease achieves effective therapy without significant systemic side-effects.

Polynucleotides may be administered by any effective method, for example, parenterally (e.g. intravenously, subcutaneously, intramuscularly) or by oral, nasal or other means which permit the polynucleotides to access and circulate in the patient's bloodstream. Polynucleotides administered systemically preferably are given in addition to locally administered polynucleotides, but also have utility in the absence of local administration. A dosage in the range of from about 0.1 to about 10 grams per administration to an adult human generally will be effective for this purpose.

The polynucleotide may be administered as a suitable genetic construct as is described below and delivered to the patient where it is expressed. Typically, the polynucleotide in the genetic construct is operatively linked to a promoter which can express the compound in the cell. The genetic constructs of the invention can be prepared using methods well known in the art, for example in Sambrook et al (2001).

Although genetic constructs for delivery of polynucleotides can be DNA or RNA, it is preferred if they are DNA. Preferably, the genetic construct is adapted for delivery to a human cell. Means and methods of introducing a genetic construct into a cell in an animal body are known in the art. For example, the constructs of the invention may be introduced into cells by any convenient method. Methods of delivering polynucleotides to a patient are well known to a person of skill in the art and include the use of immunoliposomes, viral vectors (including vaccinia (including the replication-deficient MVA strain), modified vaccinia, adenovirus and adeno-associated viral (AAV) vectors), and by direct delivery of DNA, e.g. using a gene-gun and electroporation. Furthermore, methods of delivering polynucleotides to a target tissue of a patient for treatment are also well known in the art.

Other methods involve simple delivery of the construct into the cell for expression therein either for a limited time or, following integration into the genome, for a longer time. An example of the latter approach includes liposomes (Nässander et al (1992) Cancer Res. 52, 646-653).

Other methods of delivery include adenoviruses carrying external DNA via an antibody-polylysine bridge (Curiel (1993) Prog. Med. Virol. 40, 1-18) and transferrin-polycation conjugates as carriers (Wagner et al (1990) Proc. Natl. Acad. Sci. USA 87, 3410-3414). In the first of these methods a polycation-antibody complex is formed with the DNA construct or other genetic construct of the invention, wherein the antibody is specific for either wild-type adenovirus or a variant adenovirus in which a new epitope has been introduced which binds the antibody. The polycation moiety binds the DNA via electrostatic interactions with the phosphate backbone. The adenovirus, because it contains unaltered fibre and penton proteins, is internalised into the cell and carries into the cell with it the DNA construct of the invention. It is preferred if the polycation is polylysine.

In an alternative method, a high-efficiency nucleic acid delivery system that uses receptor-mediated endocytosis to carry DNA macromolecules into cells is employed. This is accomplished by conjugating the iron-transport protein transferrin to polycations that bind nucleic acids. Human transferrin, or the chicken homologue conalbumin, or combinations thereof is covalently linked to the small DNA-binding protein protamine or to polylysines of various sizes through a disulphide linkage. These modified transferrin molecules maintain their ability to bind their cognate receptor and to mediate efficient iron transport into the cell. The transferrin-polycation molecules form electrophoretically stable complexes with DNA constructs or other genetic constructs of the invention independent of nucleic acid size (from short oligonucleotides to DNA of 21 kilobase pairs). When complexes of transferrin-polycation and the DNA constructs or other genetic constructs of the invention are supplied to the tumour cells, a high level of expression from the construct in the cells is expected.

High-efficiency receptor-mediated delivery of the DNA constructs or other genetic constructs of the invention using the endosome-disruption activity of defective or chemically inactivated adenovirus particles produced by the methods of Cotten at al (1992) Proc. Natl. Acad. Sci. USA 89, 6094-6098 may also be used. This approach appears to rely on the fact that adenoviruses are adapted to allow release of their DNA from an endosome without passage through the lysosome, and in the presence of, for example transferrin linked to the DNA construct or other genetic construct of the invention, the construct is taken up by the cell by the same route as the adenovirus particle. This approach has the advantages that there is no need to use complex retroviral constructs; there is no permanent modification of the genome as occurs with retroviral infection; and the targeted expression system is coupled with a targeted delivery system, thus reducing toxicity to other cell types.

It will be appreciated that “naked DNA” and DNA complexed with cationic and neutral lipids may also be useful in introducing the DNA of the invention into cells of the individual to be treated. Non-viral approaches to gene therapy are described in Ledley (1995, Human Gene Therapy 6, 1129-1144).

Methods of targeting and delivering therapeutic agents directly to specific regions of the body are well known to a person of skill in the art. For expression of nucleic acid molecules encoding the treatment agent, it may be useful to use monocyte/macrophage specific promoters in the vectors encoding the therapeutic polynucleotide. For example, lysM, csf1r, CD11c, CD68, macrophage SRA, and CD11b promoters are used in mice to direct the expression towards myeloid lineages.

It may also be desirable to be able to temporally regulate expression of the polynucleotide in the cell. Thus, it may be desirable that expression of the polynucleotide is directly or indirectly (see below) under the control of a promoter that may be regulated, for example by the concentration of a small molecule that may be administered to the patient when it is desired to activate or, more likely, repress (depending upon whether the small molecule effects activation or repression of the said promoter) expression of the antibody from the polynucleotide. This may be of particular benefit if the expression construct is stable, i.e., capable of expressing the compound (in the presence of any necessary regulatory molecules), in the cell for a period of at least one week, one, two, three, four, five, six, eight months or one or more years. Thus the polynucleotide may be operatively linked to a regulatable promoter. Examples of regulatable promoters include those referred to in the following papers: Rivera et al (1999) Proc Natl Acad Sci USA 96(15), 8657-62 (control by rapamycin, an orally bioavailable drug, using two separate adenovirus or adeno-associated virus (AAV) vectors, one encoding an inducible human growth hormone (hGH) target gene, and the other a bipartite rapamycin-regulated transcription factor); Magari et al (1997) J Clin Invest 100(11), 2865-72 (control by rapamycin); Bueler (1999) Biol Chem 380(6), 613-22 (review of adeno-associated viral vectors); Bohl et al (1998) Blood 92(5), 1512-7 (control by doxycycline in adeno-associated vector); Abruzzese et al (1996) J Mol Med 74(7), 379-92 (review of induction factors, e.g. hormones, growth factors, cytokines, cytostatics, irradiation, heat shock and associated responsive elements).

For veterinary use, the compound is typically administered as a suitably acceptable formulation in accordance with normal veterinary practice and the veterinary surgeon will determine the dosing regimen and route of administration which will be most appropriate for a particular animal.

In an embodiment, when a patient having an autoimmune disease is to be treated according to the first aspect of the invention, the invention further comprises administering to the patient at least one additional treatment agent that is suitable for treating that autoimmune disease. Thus the method may comprise administering to the patient a combined pharmaceutical composition containing the inhibitor of IRF5 and the further treatment agent. However, it is appreciated that the further treatment agent may be administered separately, for instance by a separate route of administration. Thus it is appreciated that the inhibitor of IRF5 and the at least one further treatment agent can be administered sequentially or (substantially) simultaneously. They may be administered within the same pharmaceutical formulation or medicament or they may be formulated and administered separately.

Current approved treatments for autoimmune disease include antagonists of TNF (e.g., etanercept, infliximab, adalimumab, certolizumab pegol, golimumab), IL1 (e.g., anakinra), IL6 (e.g., toclizumab), and IL12p40 (e.g., apilimod); inhibition of antigen presentation by CTLA4Ig (e.g., abatacept), and drugs with a less well understood role in autoimmunity (e.g., methotrexate).

In another embodiment, when a patient having cancer is to be treated according to the third aspect of the invention, the invention further comprises administering to the patient at least one additional anticancer agent. The method may comprise administering to the individual a combined pharmaceutical composition containing the IRF5 or variant thereof, or nucleic acid molecule encoding the IRF5 or variant, or agonist of IRF5, or agent that induces the expression of IRF5 in cells of the macrophage/monocyte lineage, and the further anticancer agent. However, it is appreciated that the further anticancer agent may be administered separately, for instance by a separate route of administration. Thus it is appreciated that the IRF5 or variant thereof, or nucleic acid molecule encoding the IRF5 or variant, or agonist of IRF5, or agent that induces the expression of IRF5 and the at least one further anticancer agent can be administered sequentially or (substantially) simultaneously. They may be administered within the same pharmaceutical formulation or medicament or they may be formulated and administered separately.

The further anticancer agent may be selected from alkylating agents including nitrogen mustards such as mechlorethamine (HN₂), cyclophosphamide, ifosfamide, melphalan (L-sarcolysin) and chlorambucil; ethylenimines and methylmelamines such as hexamethylmelamine, thiotepa; alkyl sulphonates such as busulphan; nitrosoureas such as carmustine (BCNU), lomustine (CCNU), semustine (methyl-CCNU) and streptozocin (streptozotocin); and triazenes such as decarbazine (DTIC; dimethyltriazenoimidazole-carboxamide); antimetabolites including folic acid analogues such as methotrexate (amethopterin); pyrimidine analogues such as fluorouracil (5-fluorouracil; 5-FU), floxuridine (fluorodeoxyuridine; FUdR) and cytarabine (cytosine arabinoside); and purine analogues and related inhibitors such as mercaptopurine (6-mercaptopurine; 6-MP), thioguanine (6-thioguanine; TG) and pentostatin (2′-deoxycoformycin); natural products including vinca alkaloids such as vinblastine (VLB) and vincristine; epipodophyllotoxins such as etoposide and teniposide; antibiotics such as dactinomycin (actinomycin D), daunorubicin (daunomycin; rubidomycin), doxorubicin, bleomycin, plicamycin (mithramycin) and mitomycin (mitomycin C); enzymes such as L-asparaginase; and biological response modifiers such as interferon alphenomes; miscellaneous agents including platinum coordination complexes such as cisplatin (cis-DDP) and carboplatin; anthracenedione such as mitoxantrone and anthracycline; substituted urea such as hydroxyurea; methyl hydrazine derivative such as procarbazine (N-methylhydrazine, MIH); and adrenocortical suppressant such as mitotane (o,p′-DDD) and aminoglutethimide; taxol and analogues/derivatives; cell cycle inhibitors; proteosome inhibitors such as Bortezomib (Velcade®); signal transductase (e.g. tyrosine kinase) inhibitors such as Imatinib (Glivec®), COX-2 inhibitors, and hormone agonists/antagonists such as flutamide and tamoxifen.

Clinically used anticancer agents are typically grouped by mechanism of action: Alkylating agents, Topoisomerase I inhibitors, Topoisomerase II inhibitors, RNA/DNA antimetabolites, DNA antimetabolites and Antimitotic agents. The US NIH/National Cancer Institute website lists 122 compounds (http://dtp.nci.nih.gov/docs/cancer/searches/standard_mechanism.html), all of which may be used in conjunction with the compound. They include Alkylating agents including Asaley, AZQ, BCNU, Busulfan, carboxyphthalatoplatinum, CBDCA, CCNU, CHIP, chlorambucil, chlorozotocin, cis-platinum, clomesone, cyanomorpholino-doxorubicin, cyclodisone, dianhydrogalactitol, fluorodopan, hepsulfam, hycanthone, melphalan, methyl CCNU, mitomycin C, mitozolamide, nitrogen mustard, PCNU, piperazine, piperazinedione, pipobroman, porfiromycin, spirohydantoin mustard, teroxirone, tetraplatin, thio-tepa, triethylenemelamine, uracil nitrogen mustard, Yoshi-864; anitmitotic agents including allocolchicine, Halichondrin B, colchicine, colchicine derivative, dolastatin 10, maytansine, rhizoxin, taxol, taxol derivative, thiocolchicine, trityl cysteine, vinblastine sulphate, vincristine sulphate; Topoisomerase I Inhibitors including camptothecin, camptothecin, Na salt, aminocamptothecin, 20 camptothecin derivatives, morpholinodoxorubicin; Topoisomerase II Inhibitors including doxorubicin, amonafide, m-AMSA, anthrapyrazole derivative, pyrazoloacridine, bisantrene HCL, daunorubicin, deoxydoxorubicin, mitoxantrone, menogaril, N,N-dibenzyl daunomycin, oxanthrazole, rubidazone, VM-26, VP-16; RNA/DNA antimetabolites including L-alanosine, 5-azacytidine, 5-fluorouracil, acivicin, 3 aminopterin derivatives, an antifol, Baker's soluble antifol, dichlorallyl lawsone, brequinar, ftorafur (pro-drug), 5,6-dihydro-5-azacytidine, methotrexate, methotrexate derivative, N-(phosphonoacetyl)-L-aspartate (PALA), pyrazofurin, trimetrexate; DNA antimetabolites including, 3-HP, 2′-deoxy-5-fluorouridine, 5-HP, alpha-TGDR, aphidicolin glycinate, ara-C, 5-aza-2′-deoxycytidine, beta-TGDR, cyclocytidine, guanazole, hydroxyurea, inosine glycodialdehyde, macbecin II, pyrazoloimidazole, thioguanine and thiopurine.

It is, however, preferred that the at least one further anticancer agent is selected from cisplatin, carboplatin, 5-fluorouracil, paclitaxel, mitomycin C, doxorubicin, gemcitabine, tomudex, pemetrexed, methotrexate, irinotecan, oxaliplatin, or combinations thereof.

When the further anticancer agent or combination of agents has been shown to be particularly effective for a specific tumour type, it may be preferred that the compound is used in combination with that further anticancer agent(s) to treat that specific tumour type.

Based upon their findings described in detail in the Examples, the inventors have identified a number of further uses of inhibitors of IRF5.

Accordingly, a fourth aspect of the invention provides a method of polarising cells of the macrophage/monocyte lineage towards the macrophage M2 phenotype, the method comprising administering to cells of the macrophage/monocyte lineage an inhibitor of IRF5.

A fifth aspect of the invention provides a method of inhibiting TNF secretion from dendritic cells (DCs), the method comprising administering an inhibitor of IRF5 to DCs.

A sixth aspect of the invention provides a method of inducing IL-10 expression and/or secretion from cells of the macrophage/monocyte lineage, the method comprising administering an inhibitor of IRF5 to cells of the macrophage/monocyte lineage.

A seventh aspect of the invention provides a method of inhibiting a Th1/Th17 immune response and/or inducing a Th2 immune response, the method comprising administering an inhibitor of IRF5 to cells of the macrophage/monocyte lineage.

For each of the fourth to seventh aspects of the invention, typically, the preferences for the inhibitor of IRF5 are as defined above with respect to the first aspect of the invention. Most preferred is the use of siRNA and adenoviral delivery of IRF5 mutants that block its activity, as discussed above.

In an embodiment of these aspects, the method may be performed on cells or tissues in vitro or ex vivo.

In alternative embodiment of these aspects, the method may be performed on cells or tissues in vivo.

In a further embodiment of these aspects, the method is performed ex vivo and the cells or tissues are subsequently administered to a patient in need thereof, such as a patient having an autoimmune disease or a Th1 polarising infection.

In specific embodiments of these aspects, the cells being treated with the IRF5 inhibitor are cells from an individual (who may be a patient having an autoimmune disease or a Th1 polarising infection or a condition associated with inflammation other than asthma or allergy), wherein the method is performed ex vivo and the cells are subsequently returned to the same individual.

This may be useful, for example, in suppressing an undesired immune or inflammatory response in the patient, such as a response related to transplant rejection. The method therefore includes aiding in the prevention of a disease or condition associated with transplant rejection such as graft versus host disease or host versus graft disease, for example in organ or skin transplants. In these cases, an inhibition or dampening of an immune or inflammatory response may be required. Thus, the invention includes the combating of transplant rejection.

Based upon their findings described in detail in the Examples, the inventors have also identified a number of further uses of IRF5. Accordingly, an eighth aspect of the invention provides a method of polarising cells of the macrophage/monocyte lineage towards the macrophage M1 phenotype, the method comprising administering to cells of the macrophage/monocyte lineage IRF5, or an agonist of IRF5, or an agent that induces the expression of IRF5 in cells of the macrophage/monocyte lineage.

A ninth aspect of the invention provides a method of inhibiting IL-10 secretion from cells of the macrophage/monocyte lineage, the method comprising administering to cells of the macrophage/monocyte lineage IRF5, or an agonist of IRF5, or an agent that induces the expression of IRF5 in cells of the macrophage/monocyte lineage.

A tenth aspect of the invention provides a method of inducing a Th1/Th17 immune response, and/or inhibiting a Th2 immune response, the method comprising administering to cells of the macrophage/monocyte lineage IRF5, or an agonist of IRF5, or an agent that induces the expression of IRF5 in cells of the macrophage/monocyte lineage.

An eleventh aspect of the invention provides a method of inducing expression of one or more genes selected from the group consisting of CXCR3, CXCR4, CXCR5, CXCR7, EBI3, TNFSF4, TNFSF9, LTA, LTB, IFN-gamma, CCL1, CCL3, CXCL5, IL-19 and IL-32 in cells of the macrophage/monocyte lineage, the method comprising administering to cells of the macrophage/monocyte lineage IRF5, or an agonist of IRF5, or an agent that induces the expression of IRF5 in cells of the macrophage/monocyte lineage.

It is appreciated that stimulating IRF5 can also be useful in the context of a vaccine because it engenders a beneficial effect on the immune system. Thus, a twelfth aspect of the invention provides IRF5, or an agonist of IRF5, for use as a vaccine adjuvant. This aspect of the invention includes a method of aiding in the vaccination of a patient, the method comprising administering IRF5, or an agonist of IRF5, to a patient who is being administered a vaccine. This aspect of the invention further includes a method of stimulating an immune response against an antigen in a patient, the method comprising administering an antigen and IRF5 or an agonist thereof, to the patient. This aspect of the invention also provides an agent that induces the expression of IRF5 in cells of the macrophage/monocyte lineage, other than GMCSF, for use as a vaccine adjuvant.

For each of the eighth to twelfth aspects of the invention, the preferences for the IRF5, the agonist of IRF5, and the agent that induces the expression of IRF5 in cells of the macrophage/monocyte lineage, are as described above with respect to the second aspect of the invention. In an embodiment, the IRF5 may be administered as a nucleic acid molecule that encodes the IRF5, for example as a vector, such as a viral vector, as described above with respect to the second aspect of the invention.

In an embodiment of these aspects, the method may be performed on cells or tissues in vitro or ex vivo.

In alternative embodiment of these aspects, the method may be performed on cells or tissues in vivo.

In a further embodiment of these aspects, the method is performed ex vivo and the cells or tissues are subsequently administered to a patient in need thereof, such as a patient having cancer, a compromised immune system, or a Th2 polarising infection.

In specific embodiments of these aspects, the cells being treated with the IRF5, the agonist of IRF5, or the agent that induces the expression of IRF5 are cells from an individual, for example a patient having cancer, a compromised immune system, or a Th2 polarising infection, wherein the method is performed ex vivo and the cells are subsequently returned to the same individual.

This may be useful, for example, in stimulating the immune system to mount a response that will be beneficial in the patient.

It is appreciated that in these aspects of the invention, when the patient has cancer, IRF5 or an agonist thereof is administered to the cells of the macrophage/monocyte lineage, and not an agent that induces the expression of IRF5.

There is a need to identify additional agents that can be used in the above aspects of the invention. For example, there is a need to identify additional inhibitors of IRF5.

Thus, a thirteenth aspect of the invention provides a method of identifying an inhibitor of IRF5, the method comprising:
providing IRF5 or a portion or a variant thereof, said portion or variant of IRF5 being capable of binding to full-length RelA (SEQ ID No: 7), and RelA or a portion or a variant thereof, said portion or variant of RelA being capable of binding to full-length IRF5 (SEQ ID No: 1);
providing a test agent; and
assessing the binding of IRF5 or said portion or a variant thereof with RelA or said portion or a variant thereof in the presence of the test agent,
wherein a test agent that interferes with IRF5/RelA binding may be an inhibitor of IRF5.

Typically, the variant of the IFR5 polypeptide has at least 90% sequence identity with the amino acid sequence of full-length human IRF5v3/4 (SEQ ID No: 1) as discussed above. Preferably, the variant of the IFR5 polypeptide has at least 91%, 92%, 93%, 94%, 95%, 96%, 97%, 98% or at least 99% sequence identity with the sequence of the IFR5v3/4 polypeptide. Such variants may be made, for example, using the methods of recombinant DNA technology, protein engineering and site-directed mutagenesis, which are well known in the art, and discussed in more detail below.

As described in Example 3, IRF5 interacts with RelA via its IRF Association Domain (IAD). Thus, in a preferred embodiment, the portion or variant of IRF5 preferably comprises the IAD domain of IRF5 which is located at residues 219-395 of IRF5v3/4. The amino acid sequence of the IRF5 IAD is denoted herein as SEQ ID No: 6.

Human RelA is a 548 amino acid residue protein whose sequence is listed in GenBank Accession No. AAH33522, revision dated 4 Aug. 2008, which is incorporated herein by reference. The amino acid sequence of human RelA is denoted herein as SEQ ID No: 7.

In an embodiment, the variant or portion of RelA has at least 90% sequence identity with full-length human RelA. Preferably, the variant of RelA has at least 91% sequence identity, or at least 92% sequence identity, or at least 93% sequence identity, or at least 94% sequence identity, or at least 95% sequence identity, or at least 96% sequence identity, or at least 97% sequence identity, or at least 98% sequence identity, or at least 99% sequence identity, with the sequence of full-length RelA polypeptide.

As described in Example 3, RelA interacts with IRF5 via its Dimerisation Domain (DD). Thus, in a preferred embodiment, the portion or variant of RelA preferably comprises or consists of the DD domain of RelAA which is located at residues 186-292 of the RelA amino acid sequence (as listed in GenBank Accession No. AAH33522, revision dated 4 Aug. 2008). The amino acid sequence of the human RelA DD is denoted herein as SEQ ID No: 8.

Such variants may be made, for example, using the methods of recombinant DNA technology, protein engineering and site-directed mutagenesis, which are well known in the art, and discussed herein. Furthermore, determining whether or not any specific variant or portion of RelA binds to full-length IRF5v3/4 is well within the ordinary ability of a person of skill in the art, for example using the methods describe below.

The inventors have also obtained experimental evidence that IRF5 binds to TRIM28 (see Example 4). Human TRIM28 is a 753 amino acid residue protein whose sequence is listed in GenBank Accession No. AAH52986, revision dated 7 Jan. 2010, which is incorporated herein by reference. The amino acid sequence of human TRIM28 is denoted herein as SEQ ID No: 9.

Thus, a fourteenth aspect of the invention provides a method of identifying an inhibitor of IRF5, the method comprising:
providing IRF5 or a portion or a variant thereof, said portion or variant of IRF5 being capable of binding to full-length TRIM28 (SEQ ID No: 9), and TRIM28 or a portion or a variant thereof, said portion or variant of TRIM28 being capable of binding to full-length IRF5 (SEQ ID No: 1);
providing a test agent; and
assessing the binding of IRF5 or said portion or a variant thereof with TRIM28 or said portion or a variant thereof in the presence of the test agent,
wherein a test agent that interferes with IRF5/TRIM28 binding may be an inhibitor of IRF5.

Preferences for the variant or portion of the IFR5 polypeptide are as described above in the previous aspect of the invention.

In an embodiment, the variant or portion of TRIM28 has at least 90% sequence identity with full-length human TRIM28 (SEQ ID No: 9). Preferably, the variant of TRIM28 has at least 91% sequence identity, or at least 92% sequence identity, or at least 93% sequence identity, or at least 94% sequence identity, or at least 95% sequence identity, or at least 96% sequence identity, or at least 97% sequence identity, or at least 98% sequence identity, or at least 99% sequence identity, with the sequence of full-length TRIM28 polypeptide. Such variants may be made, for example, using the methods of recombinant DNA technology, protein engineering and site-directed mutagenesis, which are well known in the art, and discussed herein. Furthermore, determining whether or not any specific variant or portion of TRIM28 binds to full-length IRF5v3/4 is well within the ordinary ability of a person of skill in the art, for example using the methods describe below.

Thus these aspects provide a method for selecting a compound that may be an inhibitor of IRF5, the method comprising the step of selecting a compound that interferes with IRF5/RelA binding or that interferes with IRF5/TRIM28 binding.

Since, as discussed herein, an inhibitor of IRF5 may be useful in treating an autoimmune disease, or a Th1 polarising infection, or a condition associated with inflammation other than asthma or allergy, these aspects. provide a method for selecting a compound that may be useful in treating an autoimmune disease or a Th1 polarising infection or a condition associated with inflammation other than asthma or allergy, the method comprising the step of selecting a compound that interferes with IRF5/RelA binding or that interferes with IRF5/TRIM28 binding.

Various methods may be used to determine binding between IRF5 and the RelA or TRIM28 proteins, or portions and variants thereof, including, for example, enzyme linked immunosorbent assays (ELISA), co-immunoprecipitation, copurification, surface plasmon resonance assays, chip-based assays, immunocytofluorescence, yeast two-hybrid technology and phage display which are common practice in the art and are described, for example, in Plant et al (1995) Analyt Biochem, 226(2), 342-348 and Sambrook et a/(2001). Other methods of detecting binding between IRF5 and RelA or TRIM28 or portions or variants thereof include ultrafiltration with ion spray mass spectroscopy/HPLC methods or other physical and analytical methods. Fluorescence Energy Resonance Transfer (FRET) methods, for example, well known to those skilled in the art, may be used, in which binding of two fluorescently-labelled entities (i.e. IRF5 and RelA or TRIM28 or portions or variants thereof) may be measured by measuring the interaction of the fluorescent labels when in close proximity to each other. In addition, proteomics-based identification of other interacting molecules, such as the mammalian MAPPIT system may be used to screen IRF5 expressing cells against prepared protein arrays. Thus, the test agent may be considered to be capable of interfering with the binding of IRF5 with RelA or TRIM28 if a reduction in the strength or level of binding is detected using any of these methods, as is well known in the art.

It will be appreciated that the test agent may be added to either the IRF5 protein or the portion or variant thereof before addition to the RelA or TRIM28 protein or portion or variant thereof, or it may be added to the RelA or TRIM28 protein or the portion or variant thereof before addition to the IRF5 protein or portion or variant thereof, and its affect on binding assessed.

It will be appreciated that it may be convenient to detectably label one or other of the IRF5 or the RelA or TRIM28, or the portion or variant thereof, so as to facilitate detection of their binding and consequently the effect of the test agent. Examples of suitable labels include a peptide label, a nucleic acid label (Kerr et al (1993) JACS vol. 115, p. 2529-2531; and Brenner & Lerner (1992) Proc. Natl. Acad. Sci. USA vol. 89, p. 5381-5383), a chemical label (Ohlmeyer et al (1993) Proc. Natl. Acad. Sci. USA vol. 90, p. 109222-10926; and Maclean et al (1997) Proc. Natl. Acad. Sci. USA vol. 94, p. 2805-2810); a fluorescent label (WO95/32425); and Sebestyen et al (1993) Pept. Proc. Eur. Pept. Symp. 22nd 1992, p. 63-64), or a radio frequency tag (Nicolaou et al (1995) Angew. Chem. Int. Ed. Engl. vol. 34, p. 2289-2291; and Moran et al (1995) JACS vol. 117, p. 10787-10788).

It is preferred that once a test agent has been shown to interfere with IRF5/RelA binding or IRF5/TRIM28 binding, it is subsequently tested to determine whether and to what extent it inhibits at least one function or activity of IRF5. Thus, in an embodiment, the invention includes the step of determining whether the test agent inhibits at least one function or activity of IRF5. Suitably, this includes determining any one or more of whether the test agent:
inhibits binding of IRF5 to an IRF5 binding site in DNA (and over 200 putative binding sites are shown in Supplementary Table S2 of Krausgruber et al (2011) Nature Immunology 12(3): 231-6, which is incorporated herein by reference); inhibits IRF5-mediated expression and/or secretion of TNF, IL-12, IL-23 and/or IL-1b from DCs and/or M1 macrophages; inhibits or reverses the IRF5-mediated inhibition of expression and/or secretion of IL-10 from cells of the macrophage/monocyte lineage (e.g., it may induce expression and/or secretion of IL-10 from cells of the macrophage/monocyte lineage);
inhibits IRF5-mediated upregulation of expression of one or more genes selected from the group consisting of CXCR3, CXCR4, CXCR5, CXCR7, EBI3, TNFSF4, TNFSF9, LTA, LTB, IFN-gamma, CCL1, CCL3, CXCL5, IL-19 and IL-32 in cells of the macrophage/monocyte lineage;
inhibits IRF5-mediated downregulation of expression of one or more genes selected from the group consisting of CSF1R, IL-1R2, IL1RA and TGFβ in cells of the macrophage/monocyte lineage; and
inhibits or reverses the IRF5-mediated polarisation of cells of the macrophage/monocyte lineage towards the macrophage M1 phenotype (e.g., it may polarise cells of the macrophage/monocyte lineage towards the macrophage M2 phenotype).

Another way to identify an inhibitor of IRF5, for example a drug-like compound or lead compound for the development of a drug-like compound that inhibits IRF5, is simply to contact a test agent with IRF5 or a suitable variant or fragment thereof, and determine whether, an activity or function of IRF5 is inhibited, for example reduced or eliminated, compared to the activity in the absence of the compound.

Thus a fifteenth aspect of the invention provides a method of identifying an inhibitor of IRF5, the method comprising providing a test agent, and determining whether the test agent inhibits any one or more of the following activities or functions of IRF5: the binding of IRF5 to RelA;
the binding of IRF5 to TRIM28;
IRF5-mediated expression and/or secretion of TNF from DCs;
IRF5-mediated inhibition of expression and/or secretion of IL-10 from cells of the macrophage/monocyte lineage;
IRF-mediated upregulation of expression of one or more genes selected from the group consisting of CXCR3, CXCR4, CXCR5, CXCR7, EBI3, TNFSF4, TNFSF9, LTA, LTB, IFN-gamma, CCL1, CCL3, CXCL5, IL-19 and IL-32 in cells of the macrophage/monocyte lineage;
IRF-mediated downregulation of expression of one or more genes selected from the group consisting of CSF1R, IL-1R2, IL1RA and TGFβ in cells of the macrophage/monocyte lineage; and
polarises cells of the macrophage/monocyte lineage towards the macrophage M2 phenotype.
wherein a test agent that inhibits at least one function or activity of IRF5 may be an inhibitor of IRF5.

In these screening methods, especially the method of the fifteenth aspect of the invention, it may be preferred that the test agent binds to IRF5. The test agent may be one that is predicted to bind to IRF5 by molecular modelling. Additionally or alternatively, the test agent may be one that has been shown to bind to IRF5. Thus, in an embodiment, the methods may include the prior step of predicting and/or determining whether the test agent binds to IRF5.

In these screening methods, the test agent may be any of a polypeptide, an antibody, a small molecule, a natural product, a peptidomimetic of the IAD domain of IRF5 (SEQ ID No: 6), a peptidomimetic of the DD domain of RelA (SEQ ID No: 8), or a nucleic acid. It is particularly preferred if the test agent is a small molecule (e.g. small molecule with a molecule weight less than 5000 daltons, for example less than 4000, 3000, 2000 or 1000 daltons, or with a molecule weight less than 500 daltons, for example less than 450 daltons, 400 daltons, 350 daltons, 300 daltons, 250 daltons, 200 daltons, 150 daltons, 100 daltons, 50 daltons or 10 daltons).

In many instances, high throughput screening of test agents is preferred and the method may be used as a “library screening” method, a term well known to those skilled in the art. Thus, the test agent may be a library of test agents. For example, the library may be a protein library produced, for example, by ribosome display or an antibody library prepared either in vivo, ex vivo or in vitro. Methodologies for preparing and screening such libraries are known in the art.

It is appreciated that in the screening methods described herein, which may be drug screening methods, a term well known to those skilled in the art, the test agent may be a drug-like compound or lead compound for the development of a drug-like compound.

The term “drug-like compound” is well known to those skilled in the art, and may include the meaning of a compound that has characteristics that may make it suitable for use in medicine, for example as the active ingredient in a medicament. Thus, for example, a drug-like compound may be a molecule that may be synthesised by the techniques of organic chemistry, less preferably by techniques of molecular biology or biochemistry, and is preferably a small molecule, which may be of less than 5000 daltons and which may be water-soluble. A drug-like compound may additionally exhibit improved selectivity and bioavailability, but it will be appreciated that these features may not be essential.

The term “lead compound” is similarly well known to those skilled in the art, and may include the meaning that the compound, whilst not itself suitable for use as a drug (for example because it is only weakly potent against its intended target, non-selective in its action, unstable, poorly soluble, difficult to synthesise or has poor bioavailability) may provide a starting-point for the design of other compounds that may have more desirable characteristics.

In an embodiment of these screening methods, an agent identified as a result of the initial screen may be modified and retested.

In a further embodiment of these screening methods, a compound having or expected to have similar properties to an agent identified as a result of the method may be tested.

In a still further embodiment of these screening methods, an agent that has been identified as a result of the method is tested for efficacy in a cell model of an autoimmune disease or of a Th1 polarising infection or of a condition associated with inflammation. A suitable cellular model includes a mixed lymphocyte reaction (macrophages mixed with T cells from another donor) as is well known in the art.

In a yet further embodiment of these screening methods, an agent that has been identified as a result of the method is further tested for efficacy in an animal model of an autoimmune disease and a Th1 polarising infection or of a condition associated with inflammation. Suitable animal models include animals with CIA, colitis and bacterial (e.g. E. coli) or viral (e.g., ‘flu) infections.

In a still yet further embodiment of these screening methods, an agent that has been identified as a result of the method, and having successfully completed testing in cellular and/or animal models, is further tested for efficacy and safety in a clinical trial for an autoimmune disease or a Th1 polarising infection or of a condition associated with inflammation other than asthma or allergy.

In a preferred embodiment, an agent that has been identified as a result of carrying out these screening methods is synthesised and purified. Typically, the synthesis and purification is carried out to pharmaceutically acceptable standards.

In a further preferred embodiment, an agent that has been identified as a result of carrying out these screening methods is packaged and presented for use in medicine, and preferably presented for use in treating an autoimmune disease or a Th1 polarising infection or of a condition associated with inflammation other than asthma or allergy.

A sixteenth aspect of the invention provides an IRF5/RelA complex comprising (i) IRF5 (SEQ ID No: 1) or a portion or variant thereof, said portion or variant comprising the IAD domain (SEQ ID No: 6) and being capable of binding to full-length RelA (SEQ ID No: 7), and (ii) RelA (SEQ ID No: 7) or a portion or variant thereof, said portion or variant being capable of binding to full-length IRF5 (SEQ ID No: 1).

In an embodiment, this aspect of the invention provides an IRF5/RelA complex comprising (i) IRF5 (SEQ ID No: 1) or a portion or variant thereof, said portion or variant comprising or consisting of the IAD domain (SEQ ID No: 6) and being capable of binding to full-length RelA (SEQ ID No: 7) or the RelA DD domain (SEQ ID No: 8); and (ii) RelA (SEQ ID No: 7) or a portion or variant thereof, said portion or variant comprising or consisting of the DD domain (SEQ ID No: 8) and being capable of binding to full-length IRF5 (SEQ ID No: 1) or the IAD domain (SEQ ID No: 6).

In an embodiment, one or both of (i) the IRF5 or said portion or variant thereof, and (ii) the RelA or said portion or variant thereof, in the IRF5/RelA complex are detectably labelled.

The complex may be useful in carrying out the initial screening step in the thirteenth aspect of the invention, and in carrying out a subsequent screening step in the fifteenth aspect of the invention.

A seventeenth aspect of the invention provides a kit of parts comprising (a) IRF5 (SEQ ID No: 1) or a portion or a variant thereof, said portion or variant comprising the IAD domain (SEQ ID No: 6) and being capable of binding to full-length RelA (SEQ ID No: 7), or a polynucleotide or expression vector encoding the same, and (b) RelA (SEQ ID No: 7) or a portion or variant thereof, said portion or variant being capable of binding to full-length IRF5 (SEQ ID No: 1), or a polynucleotide or expression vector encoding the same.

In an embodiment, this aspect of the invention provides a kit of parts comprising
(a) IRF5 (SEQ ID No: 1) or a portion or a variant thereof, said portion or variant comprising or consisting of the IAD domain (SEQ ID No: 6) and being capable of binding to full-length RelA (SEQ ID No: 7) or the RelA DD domain (SEQ ID No: 8), or a polynucleotide or expression vector encoding the same; and
(b) RelA (SEQ ID No: 7) or a portion or variant thereof, said portion or variant comprising or consisting of the DD domain (SEQ ID No: 8) and being capable of binding to full-length IRF5 (SEQ ID No: 1) or the IAD domain (SEQ ID No: 6), or a polynucleotide or expression vector encoding the same.

It is appreciated that such a kit of parts may be useful in a method of identifying an inhibitor of IRF5 as described above in the thirteenth and fifteenth aspects of the invention. Preferences for the IRF5 or portion or variant thereof, and the RelA or portion or variant thereof, as are described above.

An eighteenth aspect of the invention provides an IRF5/TRIM28 complex comprising (i) IRF5 (SEQ ID No: 1) or a portion or variant thereof, said portion or variant being capable of binding to full-length TRIM28 (SEQ ID No: 9), and (ii) TRIM28 (SEQ ID No: 9) or a portion or variant thereof, said portion or variant being capable of binding to full-length IRF5 (SEQ ID No: 1).

In an embodiment, one or both of (i) the IRF5 or said portion or variant thereof, and (ii) the TRIM28 or said portion or variant thereof, in the IRF5/TRIM28 complex are detectably labelled.

The complex may be useful in carrying out the initial screening step in the fourteenth aspect of the invention, and in carrying out a subsequent screening step in the fifteenth aspect of the invention.

A nineteenth aspect of the invention provides a kit of parts comprising (a) IRF5 (SEQ ID No: 1) or a portion or a variant thereof, said portion or variant being capable of binding to full-length TRIM28 (SEQ ID No: 9), or a polynucleotide or expression vector encoding the same, and (b) TRIM28 (SEQ ID No: 9) or a portion or variant thereof, said portion or variant being capable of binding to full-length IRF5 (SEQ ID No: 1), or a polynucleotide or expression vector encoding the same.

It is appreciated that such a kit of parts may be useful in a method of identifying an inhibitor of IRF5 as described above in the fourteenth and fifteenth aspects of the invention. Preferences for the IRF5 or portion or variant thereof, and the TRIM28 or portion or variant thereof, as are described above.

It is known that there is differential expression of IRF5 isoforms in autoimmune diseases, such as SLE, and these isoforms may bind to RelA and/or TRIM28 and lack one or more functions or activities of IRFv3/4. Thus a twentieth aspect of the invention provides a method of identifying a prognostic factor for an autoimmune disease, the method comprising:
determining whether, and to what extent, an IRF5 isoform other than IRF5v3/4 binds to RelA (SEQ ID No: 7) and/or TRIM28 (SEQ ID No: 9); and
determining whether the IRF5 isoform other than IRF5v3/4 lacks one or more functions or activities of IRF5v3/4,
   wherein an isoform that binds to RelA and/or TRIM28 and lacks one or more activities of IRFv3/4 may be a prognostic factor for an autoimmune disease.

In an embodiment, determining whether, and to what extent, an IRF5 isoform binds to RelA may comprise determining whether, and to what extent, an IRF5 isoform binds to the RelA DD domain (SEQ ID No: 8).

It is appreciated that the IRF5 isoforms identified to date, other than isoform 9, bind to RelA, which is consistent with our identification of the IAD domain of IRF5 as the RelA binding region. Accordingly, in an embodiment, determining whether an IRF5 isoform binds to RelA may comprise determining whether the IRF5 isoform contains the IAD domain, or may comprise determining whether the IRF5 isoform is an isoform other than isoform 9.

Preferences for the functions and activities of IRF5v3/4 to be tested are as described above.

A twenty-first aspect of the invention provides a method of identifying an inducer of IRF5 expression, the method comprising:
providing a test agent;
providing a reporter gene operably linked to an IRF5 promoter;
determining whether the test agent induces the expression of the reporter gene; and
determining whether a test agent that induces the expression of the reporter gene also induces at least one of the following functions or activities of IRF5:
   IRF5-mediated expression and/or secretion of TNF from DCs;
   IRF5-mediated inhibition of expression and/or secretion of IL-10 from cells of the macrophage/monocyte lineage;
   IRF-mediated upregulation of expression of one or more genes selected from the group consisting of CXCR3, CXCR4, CXCR5, CXCR7, EBI3, TNFSF4, TNFSF9, LTA, LTB, IFN-gamma, CCL1, CCL3, CXCL5, IL-19 and IL-32 in cells of the macrophage/monocyte lineage;
   IRF-mediated downregulation of expression of one or more genes selected from the group consisting of CSF1R, IL-1R2, IL1RA and TGFβ in cells of the macrophage/monocyte lineage; and
   IRF5-mediated polarisation of cells of the macrophage/monocyte lineage towards the macrophage M1 phenotype.

A promoter is an expression control element formed by a DNA sequence that permits binding of RNA polymerase and transcription to occur. Methods for the determination of the sequence of the promoter region of a gene are well known in the art. The presence of a promoter region may be determined by identification of known motifs, and confirmed by mutational analysis of the identified sequence. Usually, the promoter sequence is located in the region between the transcription start site and 5 kb upstream (5′) of the transcription start site of the IRF5 gene. More typically, it is located in the region between the transcription start site and 3 kb or 2 kb or 1 kb or 500 bp upstream (5′) of the start site, for example, located within the 250 by upstream (5′) of the IRF5 transcription start site. Details of the IRF5 promoter region are known in the art (see, e.g., Balasa et al, (2010) Int. J. Colorectal Dis, 25(5): 553-556; Lofgren et al (2010) J. Rheumatology 37(3): 574-578; Dideberg et al (2007) Hum. Mol. Genet. 16(24): 3008-3016; and Mancl et al (2005) J. Biol. Chem. 280(22): 21078-90).

Suitably, the reporter gene may be a gene encoding chloramphenicol acetyl transferase (CAT), luciferase, β-galactosidase or Green Fluorescent Protein (GFP) as are well known in the art.

In an embodiment of this screening method, an agent identified as a result of the initial screen may be modified and retested. Additionally or alternatively, a compound having or expected to have similar properties to an agent identified as a result of the method may be tested.

In a still further embodiment of this screening method, an agent that has been identified as a result of the method is tested for efficacy in a cell model of a condition selected from a compromised immune system, a Th2 polarising infection and cancer. Many suitable models are well known in the art.

In a yet further embodiment of this screening method, an agent that has been identified as a result of the method is further tested for efficacy in an animal model of a condition selected from a compromised immune system, a Th2 polarising infection and cancer. Many suitable animal models are well known in the art.

In a still yet further embodiment of this screening method, an agent that has been identified as a result of the method, and having successfully completed testing in cellular and/or animal models, is further tested for efficacy and safety in a clinical trial for a condition selected from a compromised immune system, a Th2 polarising infection and cancer.

In a preferred embodiment, an agent that has been identified as a result of carrying out this screening method is synthesised and purified. Typically, the synthesis and purification is carried out to pharmaceutically acceptable standards.

In a further preferred embodiment, an agent that has been identified as a result of carrying out this screening method is packaged and presented for use in medicine, and preferably presented for use in treating a condition selected from a compromised immune system, a Th2 polarising infection and cancer.

The invention will now be described in more detail with respect to the following Figures and Examples.

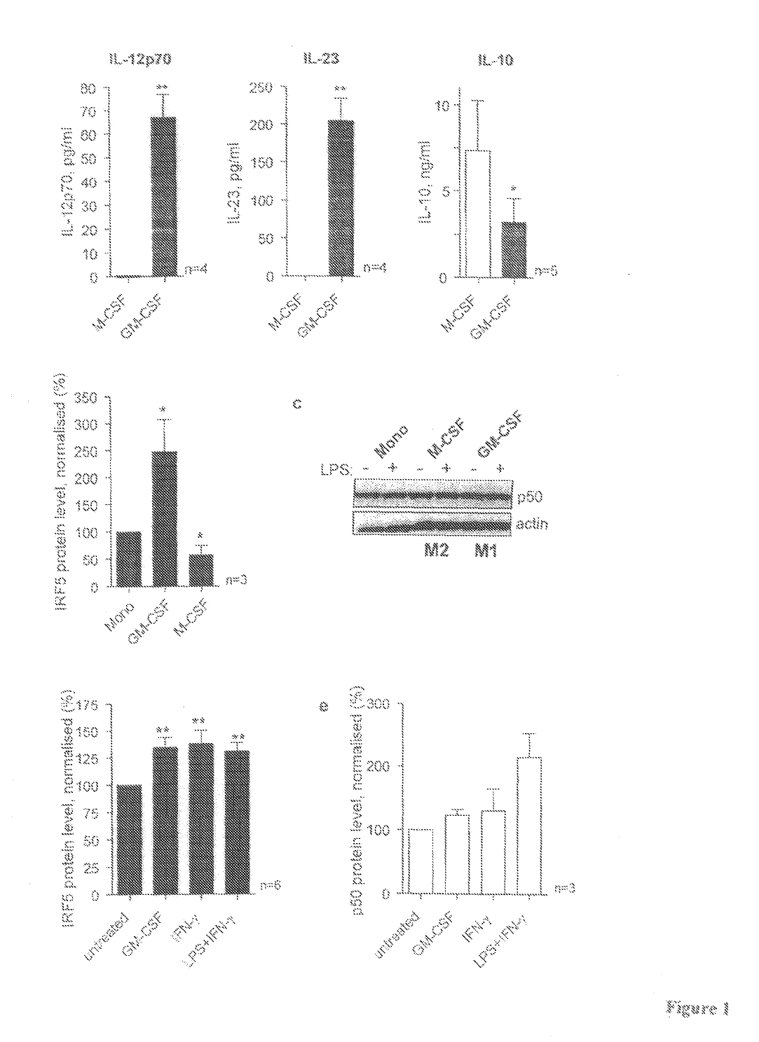

FIG. 1: IRF5 expression is induced by M1 macrophage maturation protocols

(a) M1 and M2 macrophages from the same donor were stimulated with LPS (10 ng/ml) for 24 h and the secretion of IL-12p70, IL-23 and IL-10 was determined by ELISA. Data shown are the mean±SEM from 4 independent experiments each using macrophages derived from a different donor: *p<0.05, **p<0.01 (One-way ANOVA).

(b) IRF5 protein expression was analysed in total cell lysates of monocytes, M1 and M2 macrophages by Western blotting. Densitometric analysis was performed using Quantity One software and data were normalised to actin. Shown are the mean±SEM from 3 independent experiments presented as % of increase in IRF5 protein levels relative to monocytes. *p<0.05 (One-way ANOVA with Dunnett's Multiple Comparison Post Test).

(c) p50 protein expression was analysed in total cell lysates of monocytes, M1 and M2 macrophages by Western blotting. Actin was used as a loading control. Representative blots of at least 4 independent experiments, each using cells derived from a different donor are shown.

(d, e) M2 macrophages were left untreated or treated with GM-CSF (50 ng/ml), IFN-γ (50 ng/ml), or LPS (10 ng/ml) plus IFN-γ for 24 h and total protein extracts were subjected to Western blot analysis. Densitometry was performed as in (A) and data shown are the mean±SEM from 6 independent experiments presented as % of increase in IRF5 (d) or p50 (e) protein levels relative to untreated cells. “p<0.01 (One-way ANOVA with Dunnett's Multiple Comparison Post Test).

FIG. 2: IRF5 is highly expressed in M1-like macrophages and up-regulated by GM-CSF

Total protein extracts were subjected to Western blot analysis with antibodies against IRF5, IRF4, or IRF3. Actin was used as a loading control. Representative blots of at least 4 independent experiments, each using cells derived from a different donor are shown.

(a) Monocytes (Mono) were collected at day 0 or differentiated into M1-like macrophages with GM-CSF (50 ng/ml) (GM-CSF) or M2-like macrophages with M-CSF (100 ng/ml) (M-CSF) for 5 days. Cells were either left untreated or simulated with LPS for 24 h.

(b) Monocytes were stimulated with GM-CSF (50 ng/ml) or M-CSF (100 ng/ml) for 2, 4, 8, 24 and 48 h or left untreated. The level of IRF5 mRNA was measured by RT-PCR with a corresponding TaqMan probe. Data shown are the mean±SEM of 5 independent experiments each using monocytes derived from a different donor: ***p<0.001 (Two-way ANOVA).

(c, d) For M2->M1 polarization, M2 macrophages were treated with or without GM-CSF (50 ng/ml) for 24 h. For M1->M2 polarization, M1 macrophages were treated with or without M-CSF (100 ng/ml) for 24 h. Representative blots of at least 4 independent experiments, each using cells derived from a different donor are shown

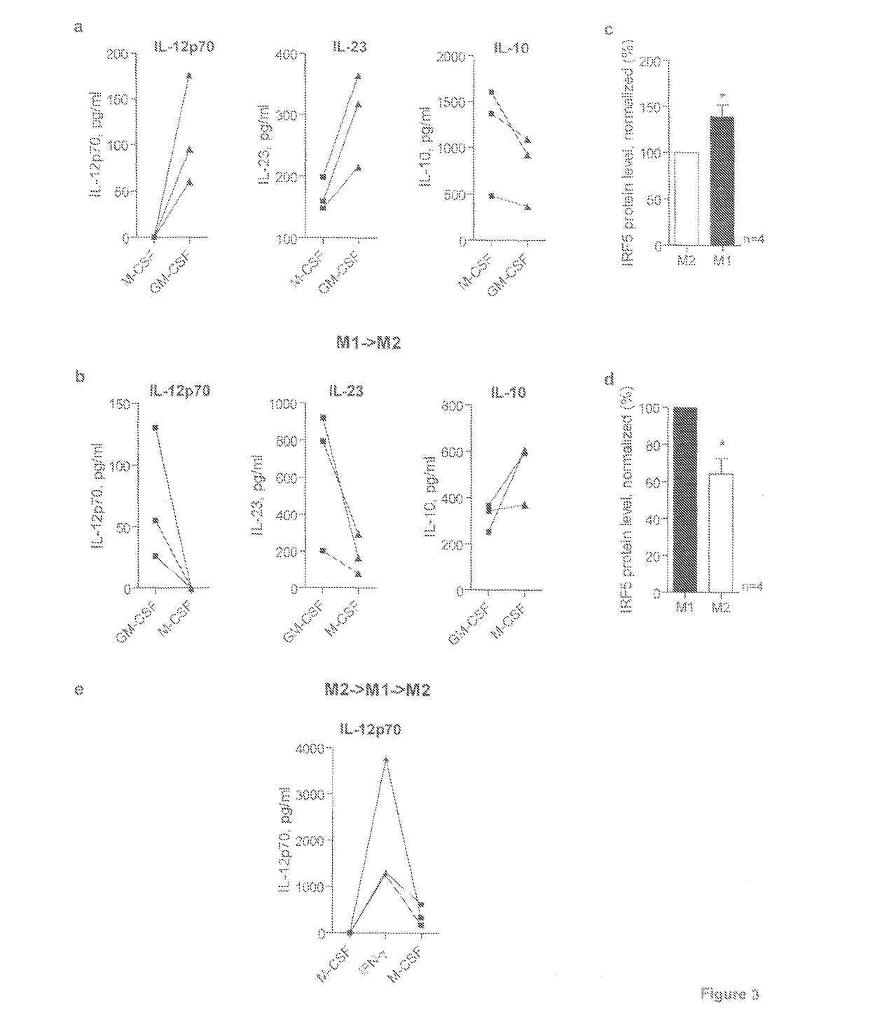

FIG. 3: Plasticity of macrophage polarization

(a, c) For M2->M1 cytokine profiles, M-CSF-derived M2 macrophages at day 5 were either left in M-CSF containing medium or exchanged for GM-CSF (100 ng/ml) containing medium and after 24 h subjected to LPS stimulation (10 ng/ml).

(b, d) For M1->M2 cytokine profiles, GM-CSF derived M1 macrophages at day 5 were either left in GM-CSF containing medium or exchanged for M-CSF (100 ng/ml) containing medium and after 24 h subjected to LPS stimulation (10 ng/ml).

(a, b) The change in secretion of IL-12p70, IL-23 and IL-10 was determined by ELISA. (c, d) The change in IRF5 protein expression was analysed by Western blotting followed by densitometric analysis using Quantity One software. The IRF5 measurements were normalised to actin. Shown are the mean±SEM from 4 independent experiments presented as % of increase (c) or decrease (d) in IRF5 protein levels relative to the initial condition: *p<0.05 (One-way ANOVA with Dunnett's Multiple Comparison Post Test).

(e) For M2->M1->M2 cytokine profiles, M2 macrophages at day 5 were either left in M-CSF containing medium, or exchanged for IFN-γ (50 ng/ml) containing medium, or further reversed to M-CSF containing medium (100 ng/ml) and after 48 h subjected to LPS stimulation (10 ng/ml).

The amount of secreted IL-12p70, IL-23 and IL-10 protein following 24 h of LPS stimulation was determined by ELISA. Data shown are the mean±SEM of 3 independent experiments each using macrophages derived from a different donor.

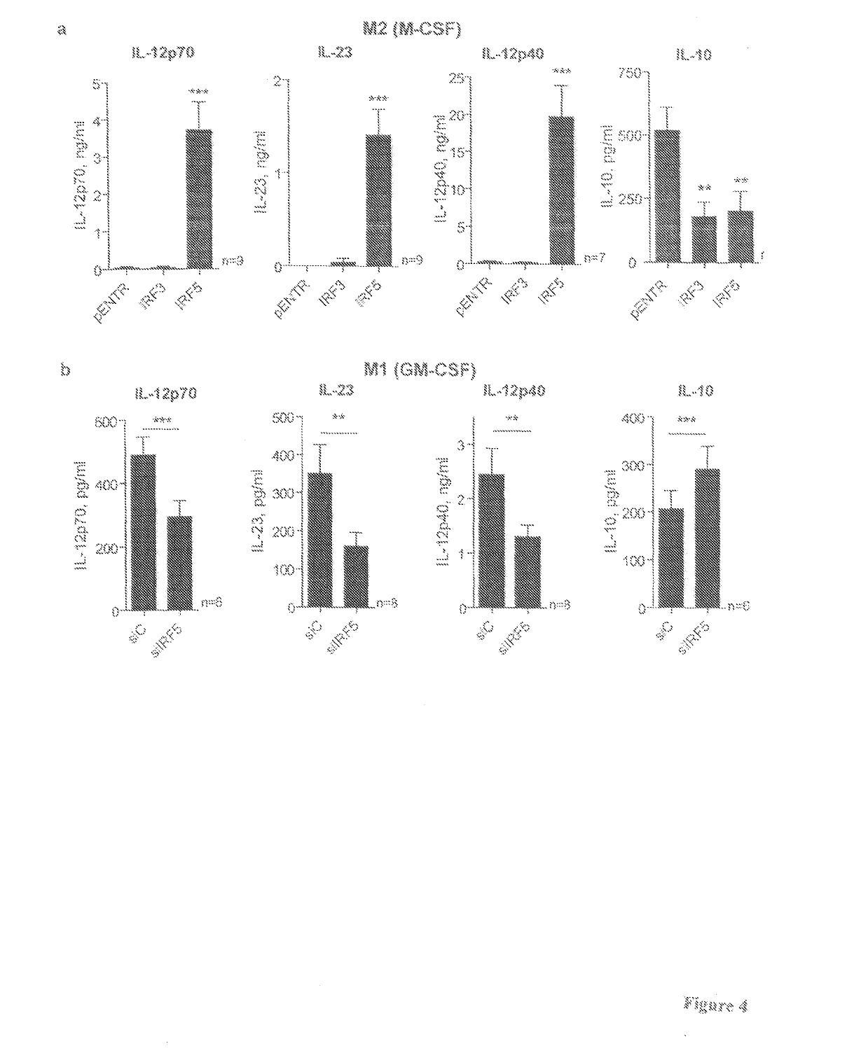

FIG. 4: IRF5 influences the production of macrophage lineage specific cytokines

(a) M2 macrophages were infected with adenoviral vectors encoding IRF5, IRF3 or empty vector (pENTR) and stimulated with LPS for 24 h. The amount of secreted IL-12p70, IL-23, IL-12p40 or IL-10 protein was determined by ELISA. Data show the trend of cytokine secretion in 7-9 independent experiments each using M2 macrophages derived from a different donor: ** p<0.01 (One-way ANOVA with Dunnett's Multiple Comparison Post Test).

(b) M1 macrophages were transfected with siRNA targeting IRF5 (siIRF5) and stimulated with LPS (10 ng/ml) plus IFN-γ (50 ng/ml) for 24 h. IL-12p70, IL-23, IL-12p40 or IL-10 secretion was compared to control cells transfected with non-targeting siRNA (siC). Data shown are the mean±SEM of 6-8 independent experiments each using M1 macrophages derived from a different donor: ***p<0.001, **p<0.01 (Student's t-test).

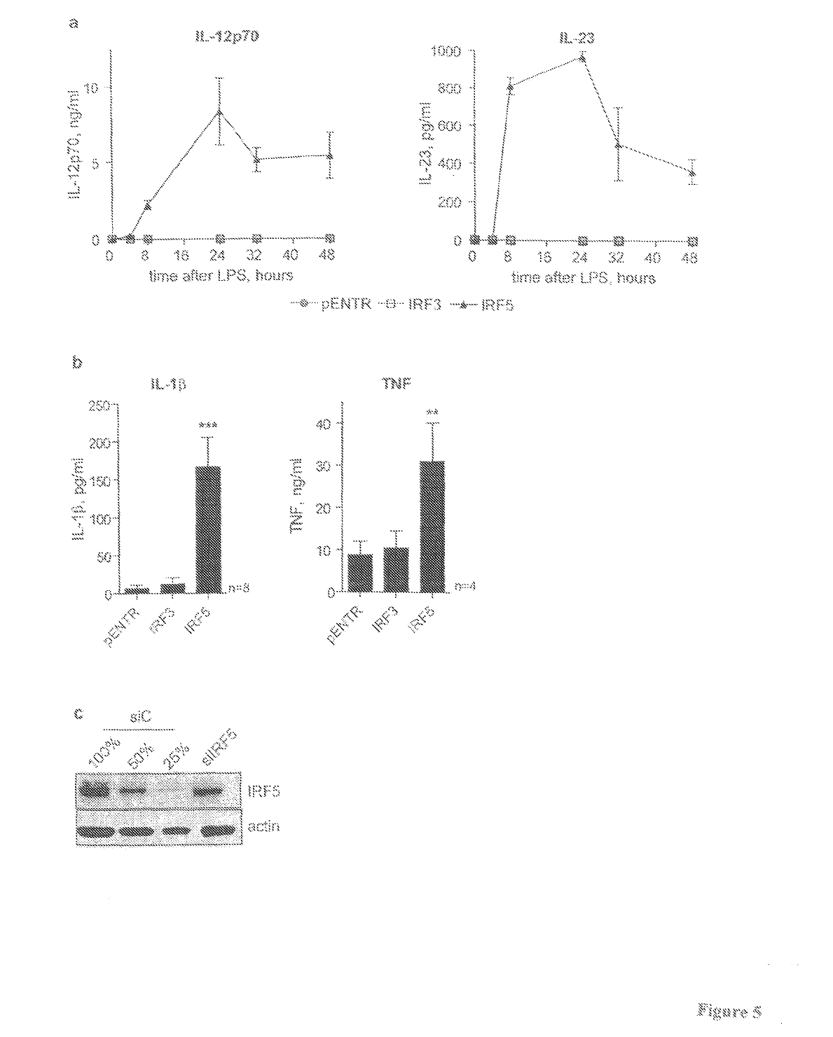

FIG. 5: IRF5 defines the production of lineage specific cytokines in human macrophages

(a) M2 macrophages were infected as in FIG. 4A and left unstimulated or stimulated with LPS (10 ng/ml) for 4, 8, 24, 32 and 48 h. The amount of secreted IL-12p70 and IL23 protein was determined by ELISA. Data shown are the mean±SD and are representative of 3 independent experiments each using macrophages derived from a different donor.

(b) M2 macrophages were infected with adenoviral vectors encoding IRF5, IRF3 or empty vector (pENTR) and stimulated with LPS for 24 h. The amount of secreted IL-Iβ and TNF protein was determined by ELISA. Data show the trend of cytokine secretion in 4-8 independent experiments each using M2 macrophages derived from a different donor: ***p<0.001, ** p<0.01 (One-way ANOVA with Dunnett's Multiple Comparison Post Test).

(c) M1 macrophages were transfected with siRNA targeting IRF5 (siIRF5) or control siRNA (siC). ˜50% of IRF5 protein was degraded estimated by serial dilutions of the siC control sample analysed by Western blotting.

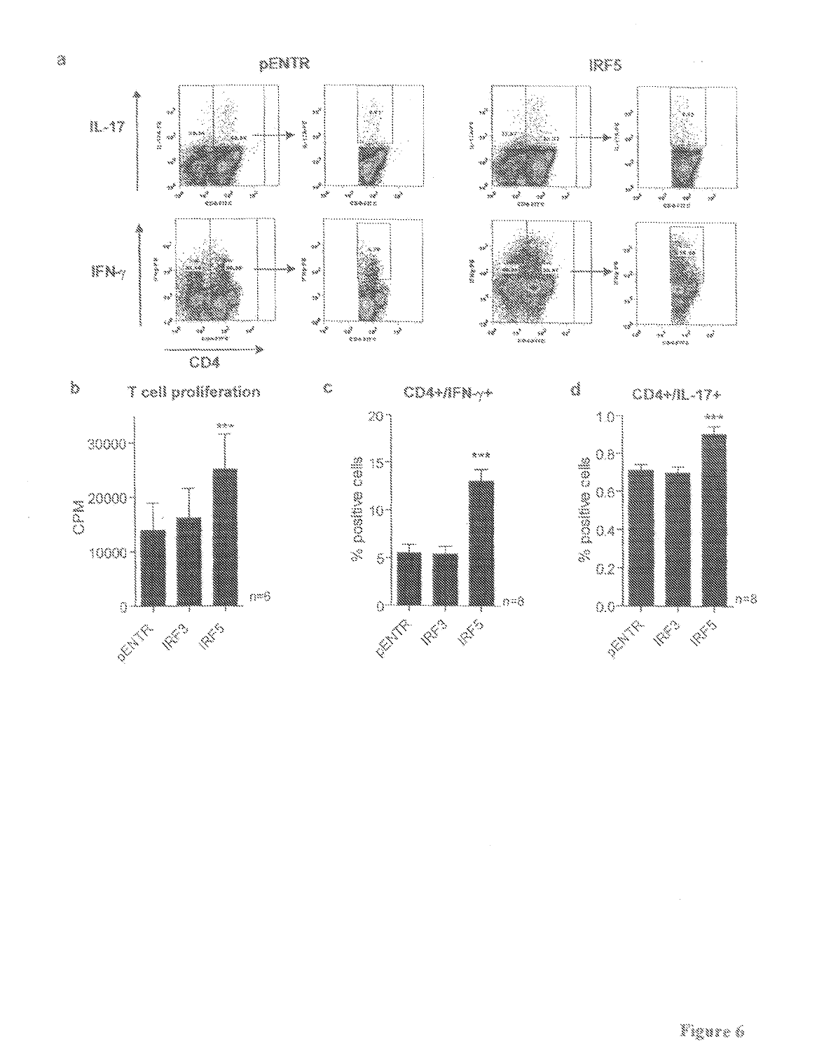

FIG. 6: IRF5 induces T cell proliferation and expression of T cell subset specific markers

(a) M2 macrophages were infected with adenoviral vectors encoding IRF5 or empty vector (pENTR) and cultured with T lymphocytes from unmatched donors. After 4 days, cells were stimulated for 3 h with PMA/ionomycine/Brefeldin A. The percentage of CD4+/IL-17+ or CD4+/IFNγ+ cells was determined by ICC staining and representative FACS plots are shown.

(b) M2 macrophages were infected with adenoviral vectors encoding IRF5, IRF3 or empty vector (pENTR) and cultured in triplicate for 72 h with T lymphocytes from unmatched donors. Cultures were pulsed with thymidine for the last 16 h to measure DNA synthesis. Control cultures contained macrophages or T-cells alone. Results are expressed as counts per minute (CPM) minus proliferation of macrophage-only cultures. Data are shown as the mean±SEM of 6 independent experiments each using cells derived from a different donor: ***p<0.001 (One-way ANOVA with Dunnett's Multiple Comparison Post Test).

(c, d) M2 macrophages were infected with adenoviral vectors encoding IRF5, IRF3 or empty vector (pENTR) and cultured with T lymphocytes from unmatched donors. After 4 days, cells were stimulated for 3 h with PMA/ionomycine/Brefeldin A and IFN-γ and IL-17 expression were determined by ICC staining. Data are shown as the percentage of IFN-γ+/IL-17− (c) or IFN-γ−/IL-17+ (d) cells±SEM of 8 independent experiments.

(e, f) M2 macrophages were infected with adenoviral vectors encoding IRF5, IRF3 or empty vector (pENTR) and cultured with T lymphocytes from unmatched donors. IFN-γ(e) or IL-17A, IL-17F, IL-21, IL-22, IL-26, IL-23R (f) mRNA expression was analysed after 2 days of co-culture. Data are shown as the mean±SEM of 6-9 independent experiments each using cells derived from a different donor: *p<0.05, **p<0.01, ***p<0.001 (One-way ANOVA with Dunnett's Multiple Comparison Post Test).

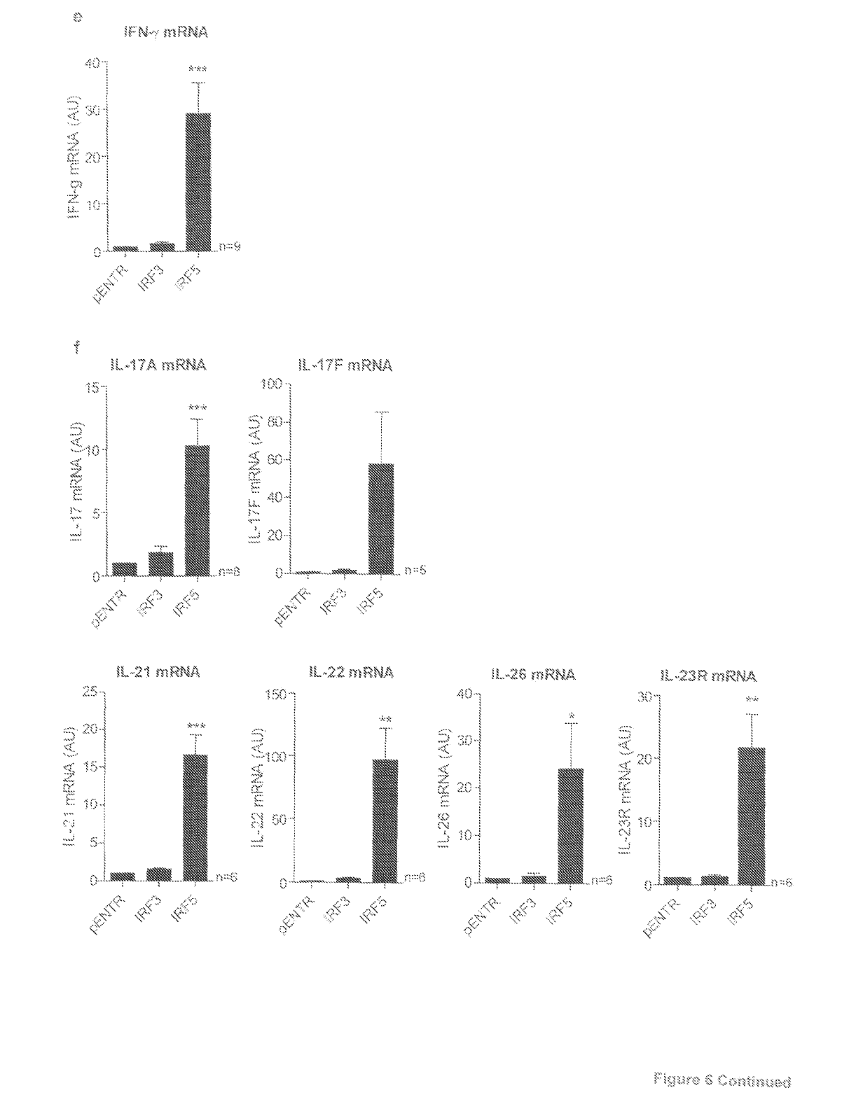

FIG. 7: IRF5 promotes lymphocyte proliferation and Th1/Th17 response

(a, d) M2-like macrophages were infected with adenoviral vectors encoding IRF5, IRF3 or empty vector (pENTR) and cultured with T lymphocytes from unmatched donors. After 4 days, cells were stimulated for 3 h with PMA/ionomycine/Brefeldin A and IFN-γ and IL-17 expression were determined by ICC staining. Data are shown as the mean fluorescence intensity (MFI)±SEM of 7 independent experiments.

(b, e) Supernatants after 4 days of co-culture were analysed for IFN-γ (b) and IL-17A (d) production. Data are shown as the mean±SEM of 6 (b) or 4 (c) independent experiments. ˜200 pg/ml of IFN-γ and no detectable IL-17 was produced by M2 macrophages infected with adenoviral vectors encoding IRF5, IRF3 or empty vector (pENTR).

(c, f) M2-like macrophages were infected with adenoviral vectors encoding IRF5, IRF3 or empty vector (pENTR) and cultured with T lymphocytes from unmatched donors. T-bet (c) and RORyT (f) mRNA expression was analysed after 2 days of co-culture. Data are shown as the mean±SEM of 6 independent experiments each using cells derived from a different donor: *p<0.05, **p<0.01, ***p<0.001 (One-way ANOVA with Dunnett's Multiple Comparison Post Test).

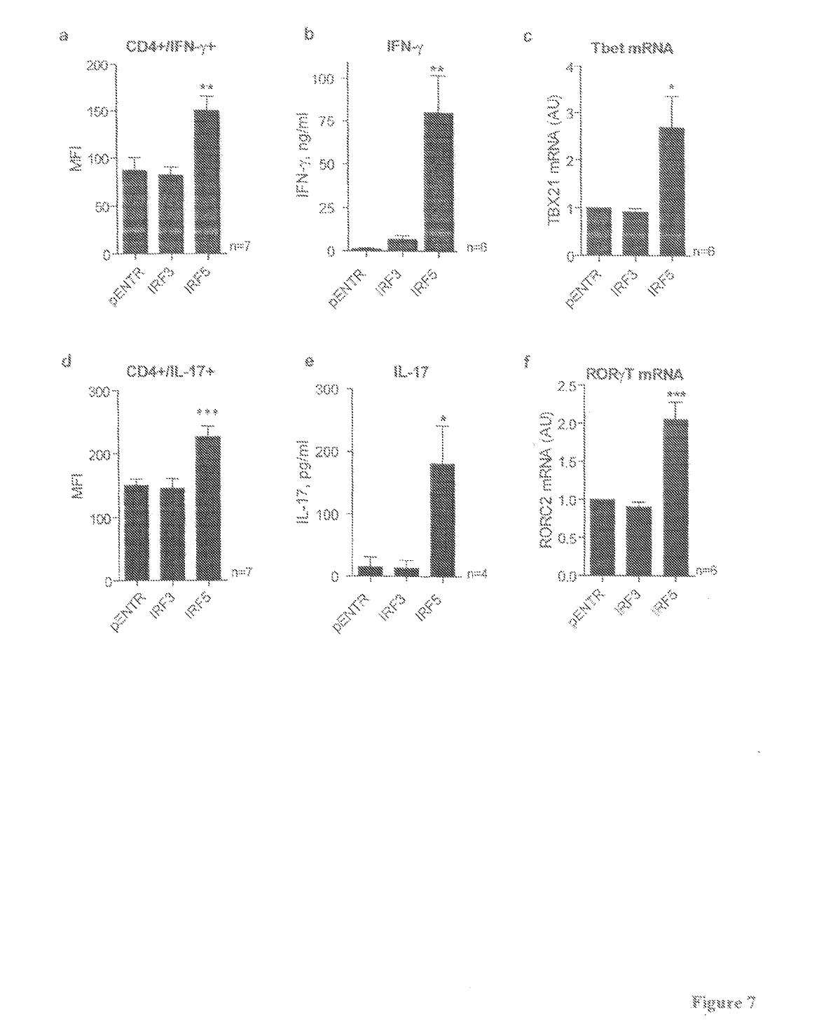

FIG. 8: IRF5 regulates mRNA expression of macrophage lineage specific cytokines

(a) M2 macrophages were infected with adenoviral vectors encoding IRF5 or IRF3 and basal cytokine mRNA expression was compared to empty vector (pENTR) control infected cells. IL-12p40, IL-12p35, IL-23p19 or IL-10 mRNA levels in unstimulated cells were analysed by q-PCR. Data shown are the mean±SEM of 3-6 independent experiments each using M2 macrophages derived from a different donor: *p<0.05, **p<0.01, ***p<0.001 (One-way ANOVA with Dunnett's Multiple Comparison Post Test).

(b) M1 macrophages were transfected with siRNA targeting IRF5 (siIRF5) and stimulated with LPS (10 ng/ml) for 8 h or left untreated (IL-10). IL-12p40, IL-12p35, IL-23p19 or IL-10 mRNA expression was compared to control cells transfected with non-targeting siRNA (siC). Data shown are the mean±SEM of 5-6 independent experiments presented as a % of reduction in cytokine mRNA levels by siIRF5: ***p<0.001, **p<0.01 (Student's t-test).

(c) M2-macrophages from 4 different donors were infected with adenoviral vectors encoding IRF5 or empty vector (pENTR) and global mRNA expression was analysed using Illumina HumanHT-12 Expression BeadChips. Heatmaps showing the fold change in M2+IRF5 cells relative to M2 cells at 0 hr for sets of M1 and M2-specific genes described in 21,27. Red indicates higher expression in M2+IRF5 and green indicates higher expression in M2 (scale shows the log 2 fold change). M1-specific genes tend to be more highly expressed in M2+IRF5 cells whereas M2-specific genes are downregulated by IRF5.

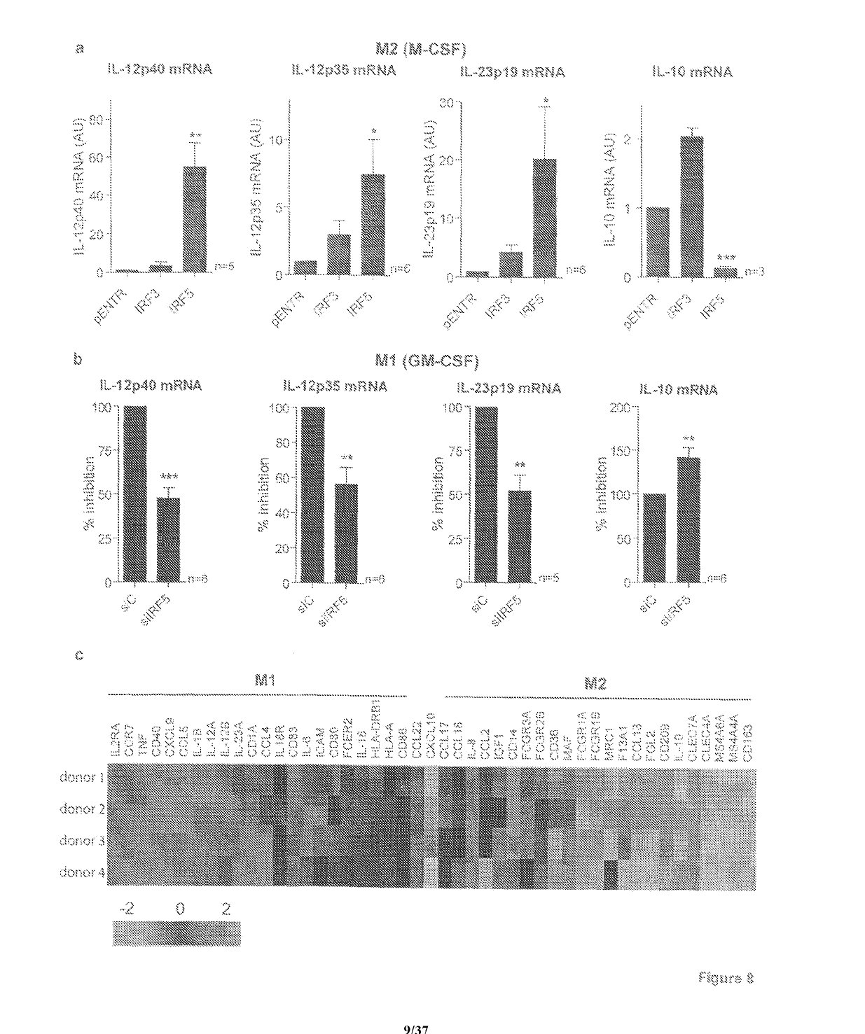

FIG. 9: IRF5 drives expression of IL12p40 mRNA and production of selected M1 and M2 cytokines

(a) M2 macrophages were infected with adenoviral vectors encoding IRF5 or empty vector (pENTR) and left unstimulated or stimulated with LPS (10 ng/ml) for 4, 8, 16 and 24 h. IL-12p40 mRNA expression was compared to unstimulated pENTR control cells. Data shown are the mean±SD and are representative of 3 independent experiments each using macrophages derived from a different donor.

(b) M1 macrophages were transfected with siRNA targeting IRF5 (siIRF5) or control siRNA (siC) and left unstimulated or stimulated with LPS (10 ng/ml) for 2, 4, 8, 16 and 24 h. IL-12p40 mRNA expression was compared to control cells transfected with non-targeting siRNA (siC). Data shown are the mean±SD of representative experiments presented as a % of reduction in IL-12p40 mRNA levels by siIRF5.

(c, d, e) M2 macrophages were infected with adenoviral vectors encoding IRF5 or empty vector (pENTR) and stimulated with LPS for 24 h. The amount of secreted CCLS (c); CCL2, CCLI3 (d) or CCL22, CXCLIO (e) protein was determined by ELISA. The amount of CD40 (c) or CD163 (c) surface expression was determined by FACS and expressed as MFI. Data are shown as the mean±SEM of 4-6 independent experiments each using M2 macrophages derived from a different donor: ** p<0.01, *p<0.05 (Student's t-test).

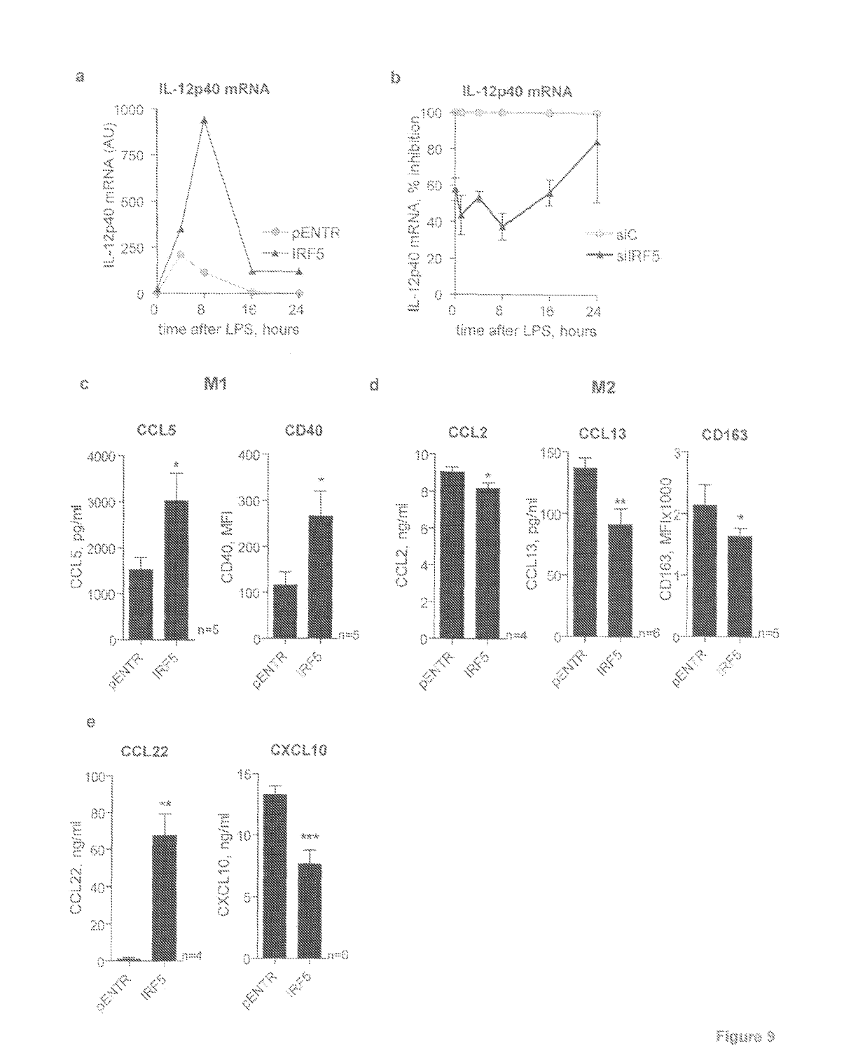

FIG. 10: Genes newly-identified as being up- or down-regulated by IRF5

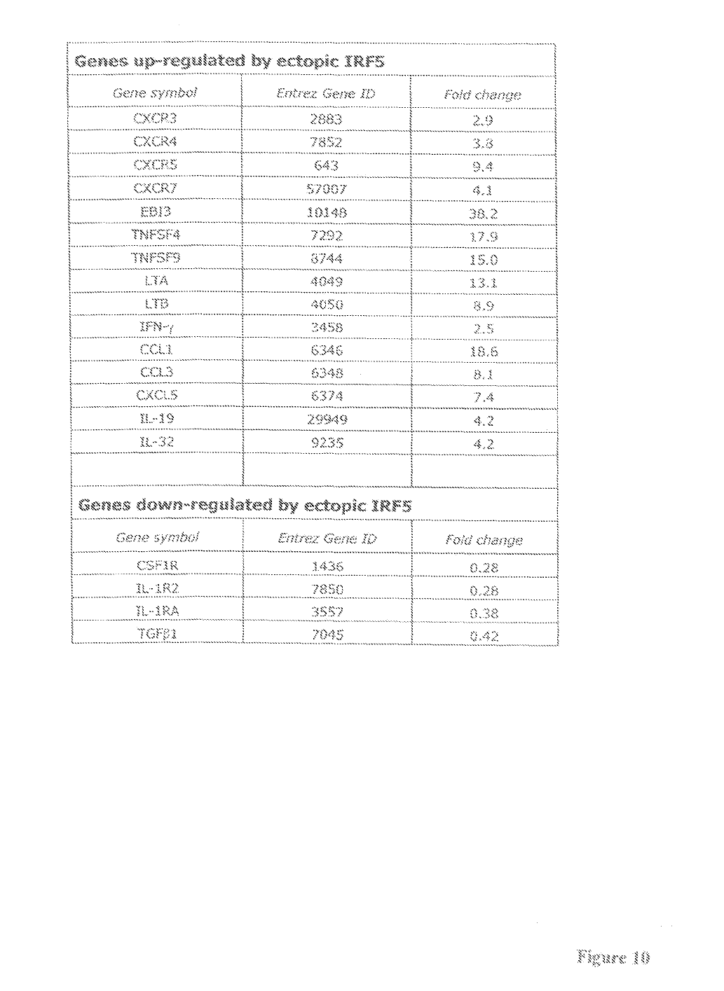

FIG. 11: IRF5 is directly involved in transcriptional regulation of lineage specific cytokines

(a-d) M1 macrophages were left unstimulated or stimulated with LPS (10 ng/ml) for 1, 2, 4, 8 or 24 h followed by ChIP with antibodies specific to IRF5 (black bars), PolII (grey bars), or IgG control (white bars). Protein recruitment to the promoters of IL-12p40 (a), IL-12p35 (b), IL-23p19 (c) or IL-10 (d) was measured and presented as mean % input relative to genomic DNA (gDNA)±SD of a representative experiment.

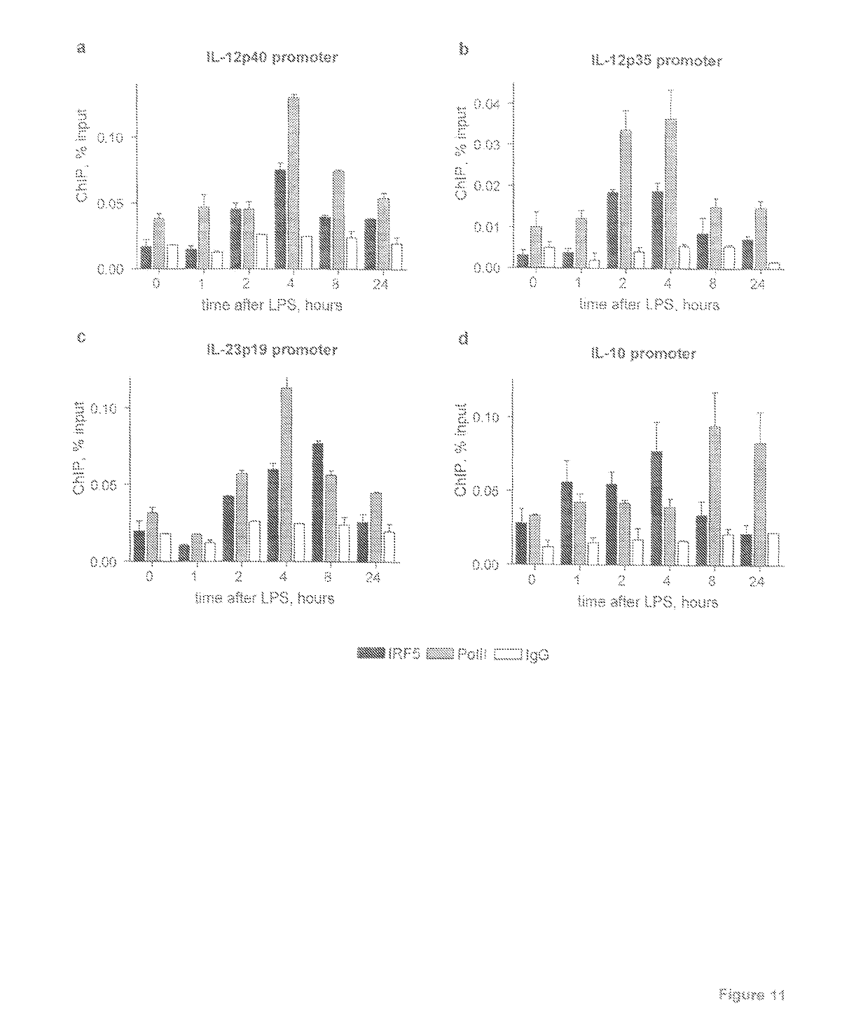

FIG. 12: IRF5 inhibits transcriptional activation of the human IL-10 gene

M2 macrophages were co-infected with (a) IL-10 wild type (IL-10-Luc wt) reporter plasmid or (b) the IL-10 plasmid in which site-specific mutations were introduced into the ISRE site at −180 to −173 (IL-10-Luc ISRE mut) and constructs coding for IRF5 (black bars), IRF5 DNA-binding mutant (IRF5ΔDBD) (grey bars) or empty vector (pENTR) (white bars). 24 h post-infection, cells were left unstimulated or stimulated with LPS (10 ng/ml) for 4 h and luciferase activity was measured. Data are presented as the mean±SEM from 3 independent experiments each using M2 macrophages derived from a different donor: **p<0.01 (One-way ANOVA with Dunnett's Multiple Comparison Post Test).

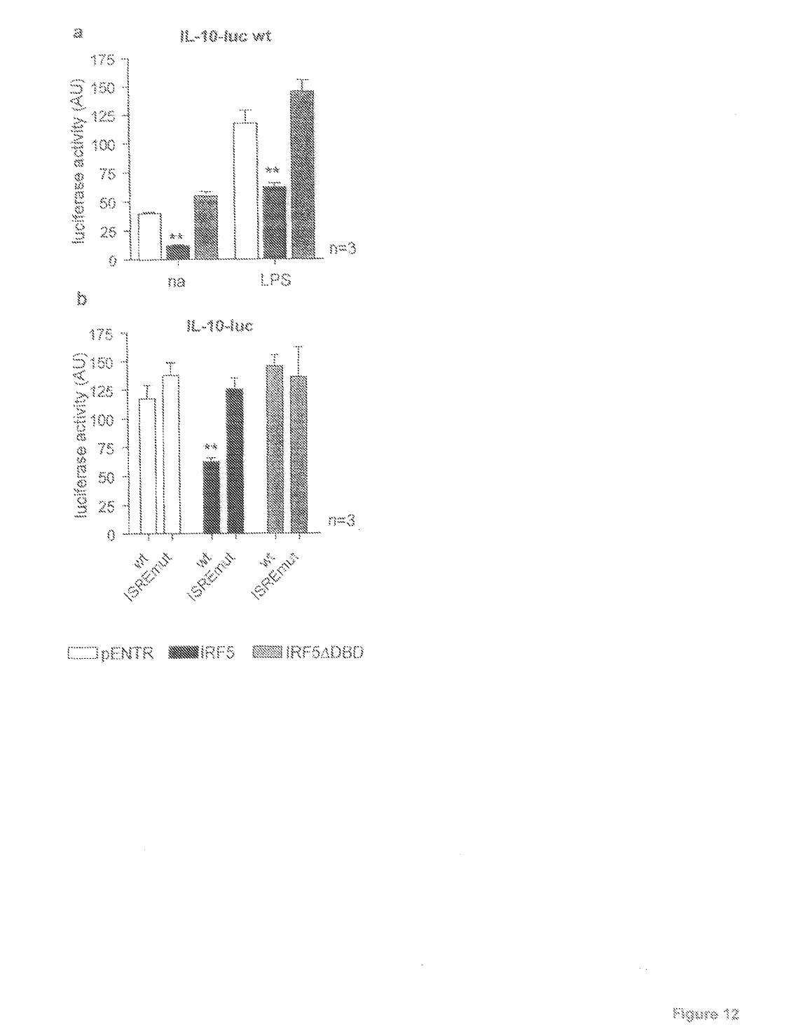

FIG. 13: IRF5 activates transcription of the human II-12p3S gene HEK-293-TLR4jMD2 cells were co-transfected with IL-12p35 wild type (IL-12p35-Luc wt) reporter plasmid or the IL-12p35 plasmid in which site-specific mutation was introduced into the ISRE site as described in Ref 37 and constructs coding for IRF5 (black bars), IRF5 DNA-binding mutant (IRF5ΔDBD) (grey bars) or empty vector (pENTR) (white bars). Luciferase activity was measured 24 h post-infection. Data are presented as the mean±SD from a representative out of 3 independent experiments.

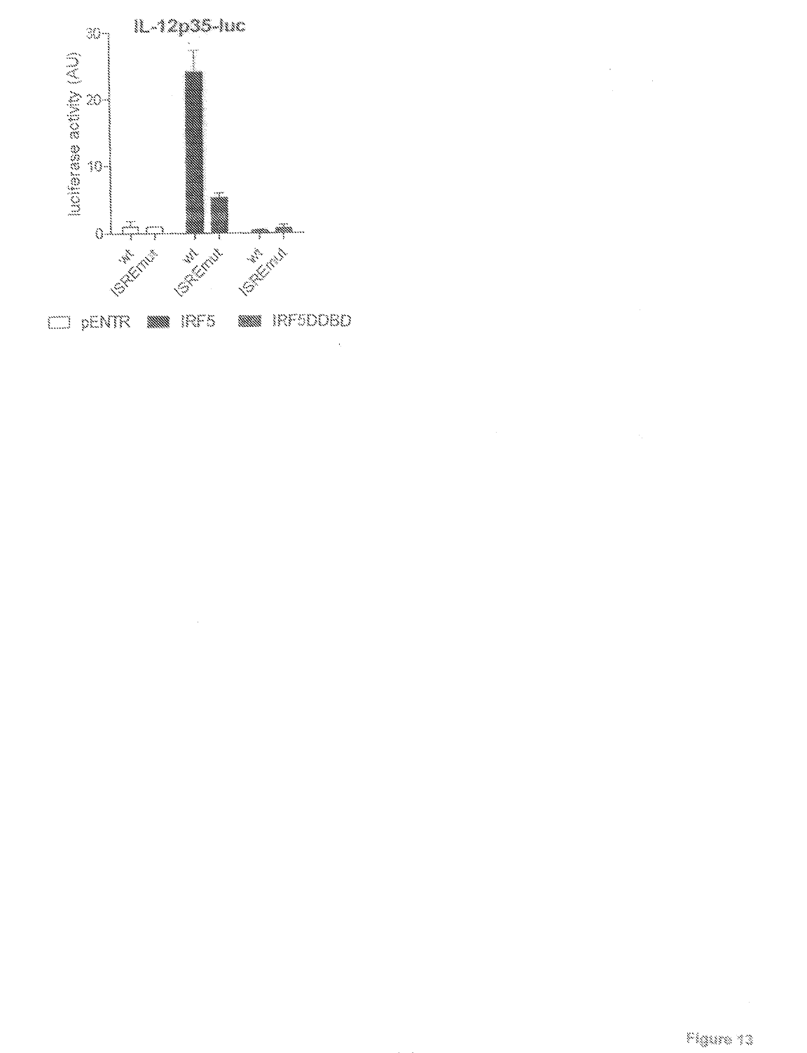

FIG. 14: Impaired transcription of M1 and Th1/Th17 cytokines in Irf5−/− mice

(a) Bone-marrow cells from C57BL/6 mice were differentiated into M1 macrophages with GM-CSF (50 ng/ml). On day 8, total protein extracts from adherent cells were subjected to Western blot analysis with antibodies against IRF5.

(b) M1 macrophages were stimulated with LPS (100 ng/ml) for 24 h and the amount of secreted 1′-12p70, 11-23 and 11-10 was determined by ELISA.

(c) Littermate wild type (n=10) and irf5−/− (n=10) mice were intraperitoneally injected with LPS (20 ug/ml). Mice were sacrificed after 3 h and serum concentrations of Il-12p40, Il-23 and Il-10 were measured by either ELISA (il-12p40, Il-23) or BDTM cytometric bead assay (Il-10). Data are shown as the mean±SEM of 8-10 serum samples from 3 independent experiments: ** p<0.01, *p<0.05 (Student's t-test).

(d) mRNA levels of selected M1 and M2 markers were analysed in peritoneal cells from LPS-injected mice in (c). Data are shown as the mean±SEM of 11 samples from 3 independent experiments: *** p<0.001, ** p<0.01, *p<0.05 (Student's t-test).

(e) Spleen cells from LPS-injected mice in (c) were cultured in the presence of anti-CD3 antibodies for 48 h. The amount of secreted Ifn-γ and II-17a was determined by ELISA. Data are shown as the mean±SEM of 4-5 spleen cultures from two independent experiments: ** p<0.01, *p<0.05 (Student's t-test).

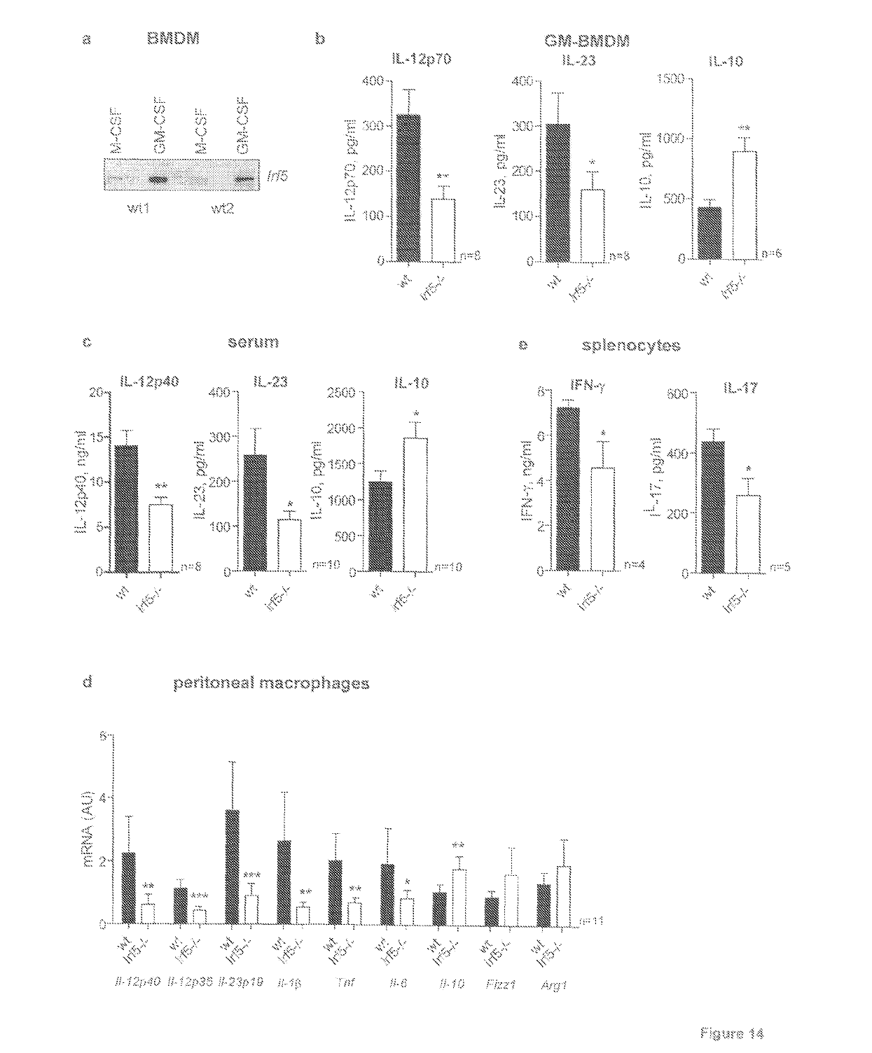

FIG. 15: Impaired production of M1 cytokines in Irf5−/− mice

Littermate wild type (n=10) and irf5−/− (n=10) mice were intraperitoneally injected with LPS (20 ug/ml). Mice were sacrificed after 3 h and serum concentrations of Il-1β, Il-6 and Tnf were measured by BDTM cytometric bead assay. Data are shown as the mean±SEM of 8-10 serum samples from 3 independent experiments: ** p<0.01, *p<0.05 (Student's t-test).

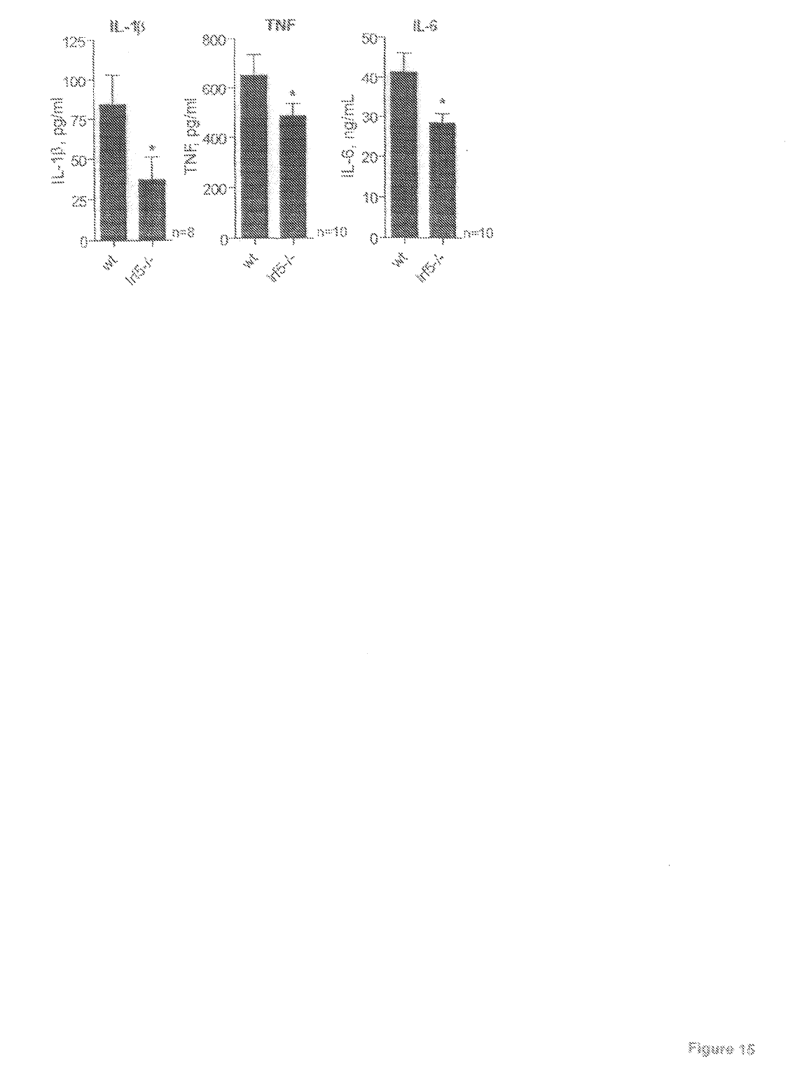

FIG. 16: IRF5 in experimental models of arthritis.

(A) Male DBA mice were intradermally immunised with bovine CII in CFA and IRF5 mRNA expression in the affected paws was measured at days 1 and 10 post-onset of arthritis. (B,C) Male C57BL/6 Irf5−/− and wild type littermates were subcutaneously immunised with mBSA followed by intra-articular injection of mBSA or PBS into the right or left knee joint, respectively. IRF5 (B) and cytokine (C) mRNA expression in mBSA and PBS treated knees was measured 2 days after intra-articular injection.

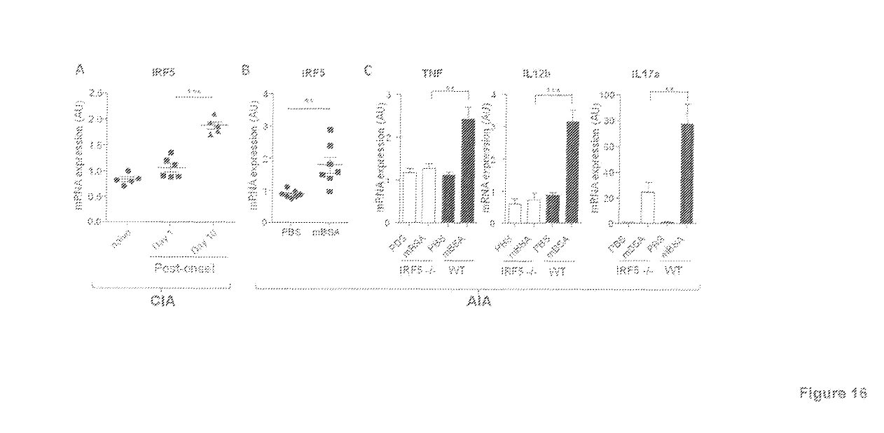

FIG. 17: IRF5 protein is highly expressed in MDDCs and controls late phase TNF secretion

(A) MDDCs and MDMs were stimulated with LPS (10 ng/ml) for 4 h and 24 h and secreted TNF was measured by ELISA. Data show mean±standard error of the mean (SEM) of 5 independent experiments each using monocytes derived from a different donor: *p<0.05, ** p<0.01 (Student's T-test).

(B) MDDCs were stimulated with LPS for 2 h and then were cultured with T lymphocytes and anti-TNFR1 or anti-IgG control antibodies were added 6 h or 24 h after co-culture start. IFN-γ secretion was determined by ELISA after 72 h of co-culture. Data show mean±SEM of 3 independent experiments.

(C) Cells were collected at day 0 (monocytes); day 1, 3, 5 and 7 (MDDCs) post differentiation with GM-CSF (50 ng/ml) and IL-4 (10 ng/ml); day 1, 3 and 5 post differentiation with M-CSF (50 ng/ml) (MDMs) and total protein extracts were subjected to Western blot analysis. p38 MAPK was used as loading control. Representative blots of 5 independent experiments each using monocytes derived from a different donor.

(D) MDMs were left untreated (cells) or infected with adenoviral vectors encoding IRF5 or empty vector (pBent), stimulated with LPS for 2 h and cultured with T lymphocytes. IFN-γ secretion was determined by ELISA after 72 h of co-culture. Data show mean±standard deviation (SD) and are representative of 3 independent experiments each using MDMs derived from a different donor.

(E) MDMs were left untreated (cells) or infected with adenoviral vectors encoding IRF5, IRF3 or empty vector (pBent) and stimulated with LPS for the indicated time. The amount of secreted TNF protein was determined by ELISA. Data show mean±SD and are representative of 3 independent experiments each using MDMs derived from a different donor.

(F) MDDCs were transfected with siRNAs targeting IRF5 (siIRF5) and stimulated with LPS (10 ng/ml) for the indicated time. TNF secretion was compared to control cells transfected with non-targeting siRNA (siC). Data shown are the mean±SD and are representative of 2 independent experiments each using MDDCs derived from a different donor.

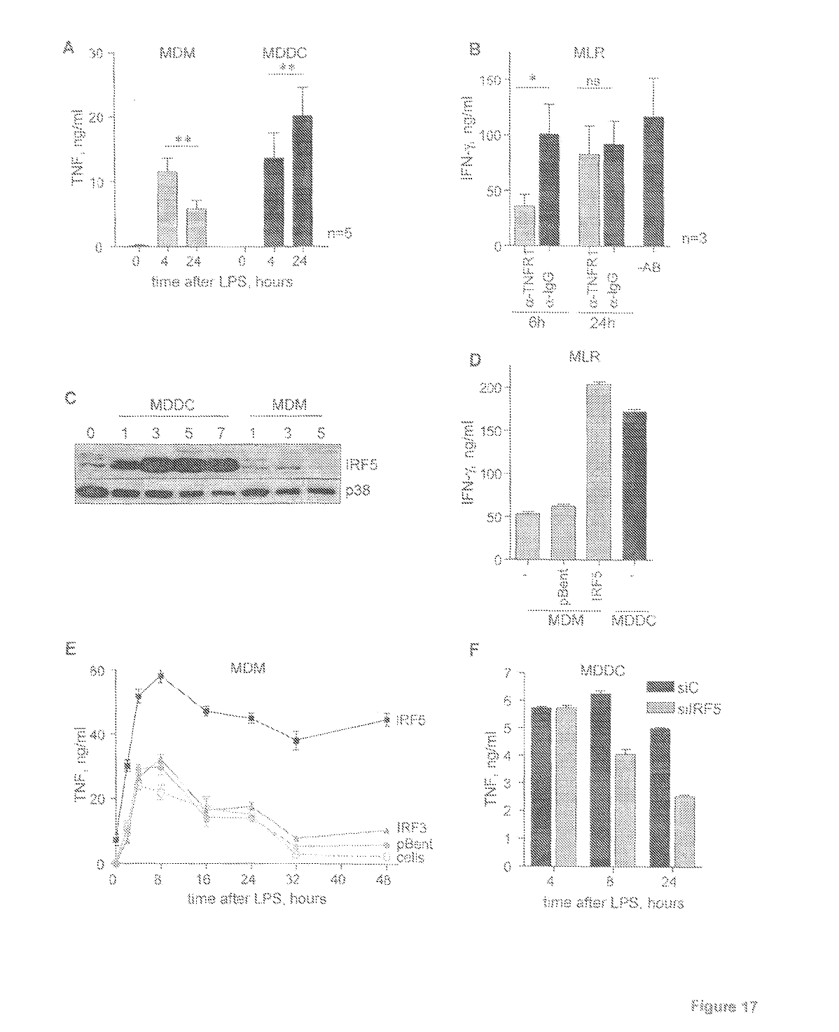

FIG. 18: Secretion of TNF is sustained in MDDCs or in IRF5-induced MOMs

(A) MDDCs and MDMs were stimulated with LPS (10 ng/ml) for 4 and 24 h and secreted TNF was measured by ELISA. Data show the trend in TNF secretion in 5 (MDMs) or 6 (MDDCs) donors.

(B) MDMs were infected with adenovirus coding for IRF5-HA and IRF3-HA The expression of each construct was determined 48 h post-infection by subjecting equal amounts of whole cell protein lysates to western blot analysis and probing with anti-HA antibodies.

(C) MDMs were infected with adenovirus coding for IRF5, IRF3 or empty vector (pBENT) and the amount of secreted TNF protein in unstimulated cells was measured by ELISA. Data show 9 independent experiments each using monocytes derived from a different donor.

(D) The 8 h post LPS supernatants from (B) were analysed by ELISA for IFN-AI secretion. Data show 2 independent experiments each using monocytes derived from a different donor.

(E) MDDCs were transfected with siRNAs targeting IRF5 (siIRF5) or control siRNA (siC). −50% of IRF5 protein was degraded estimated by serial dilutions of the siC control sample analysed by Western blotting.

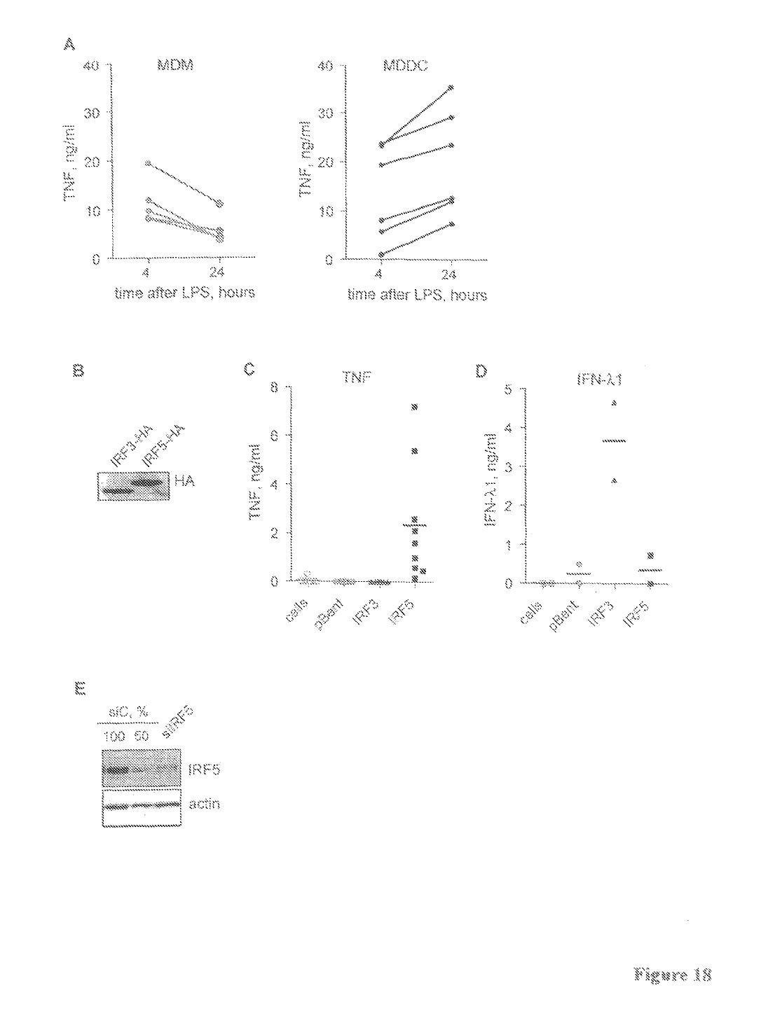

FIG. 19: IRF5 is involved in transcriptional regulation of TNF

(A) MDDCs were transfected with siRNAs targeting IRF5 (siIRF5), RelA (siRelA) or both (si(IRF5+RelA)) and stimulated with LPS (10 ng/ml) for the indicated time. TNF mRNA expression was compared to control cells transfected with non-targeting siRNA (siC).

Data shown are the mean±SD and are representative of 4 independent experiments each using MDDCs derived from a different donor.

(B) HEK-293 cells were co-transfected with the TNF 5′ wt/3′ wt reporter plasmid and equal amounts of expression plasmids encoding for human IRF5, RelA, IRF3 or empty vector (pBent). 48 h post-transfection cells were harvested and luciferase activity was measured as described. Data are presented as a fold over pBent±SEM from 4 independent experiments: *p<0.05, ** p<0.01 (One-way ANOVA).

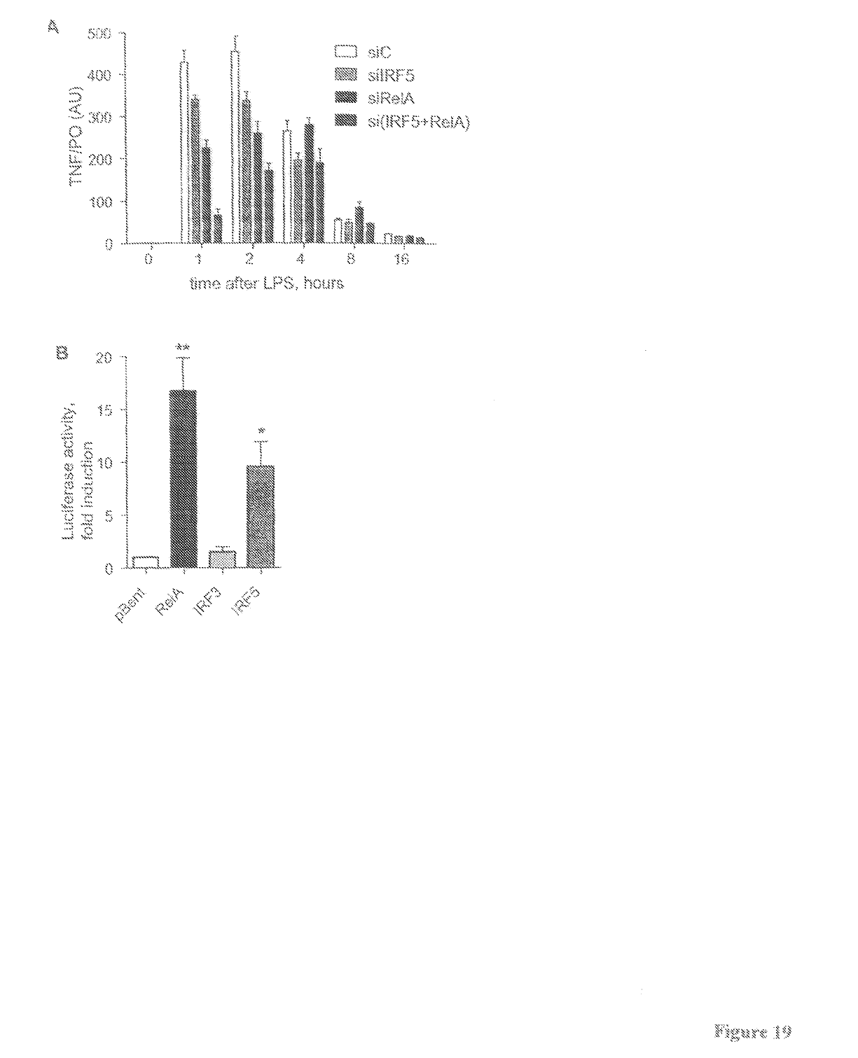

FIG. 20: LPS-induced expression of TNF mRNA is IRF5 dependent

(A) MODCs and MDMs from the same donor were stimulated with LPS for the indicated time and TNF mRNA expression was determined by 2-standard curve RT-PCR. Data shown are from a representative experiment.

(B) MDDCs were transfected with siRNAs targeting IRF5 (siIRF5) or control siRNA (siC). −60% of IRF5 mRNA was degraded and affected LPS-induced TNF mRNA expression in MDDCs. Data shown are the mean±SEM of 4 independent experiments presented as a % of reduction in TNF mRNA levels by siIRF5: *p<0.05, ** p<0.01 (Student's T-test).

(C) HEK-293 cells were co-transfected with the TNF 5′ wt/3′ wt reporter plasmid and equal amounts of expression plasmids encoding NF-κB subunits or empty vector (pBent).

(D) HEK-293 cells were co-transfected with the TNF 5′ wt/3′ wt reporter plasmid and equal amounts of expression plasmids encoding IRF5, IRF5 IDBD, IRF5 A68P or empty vector (pBent).

(C,D) Data are presented as a fold over pBent±SD from a representative experiment.

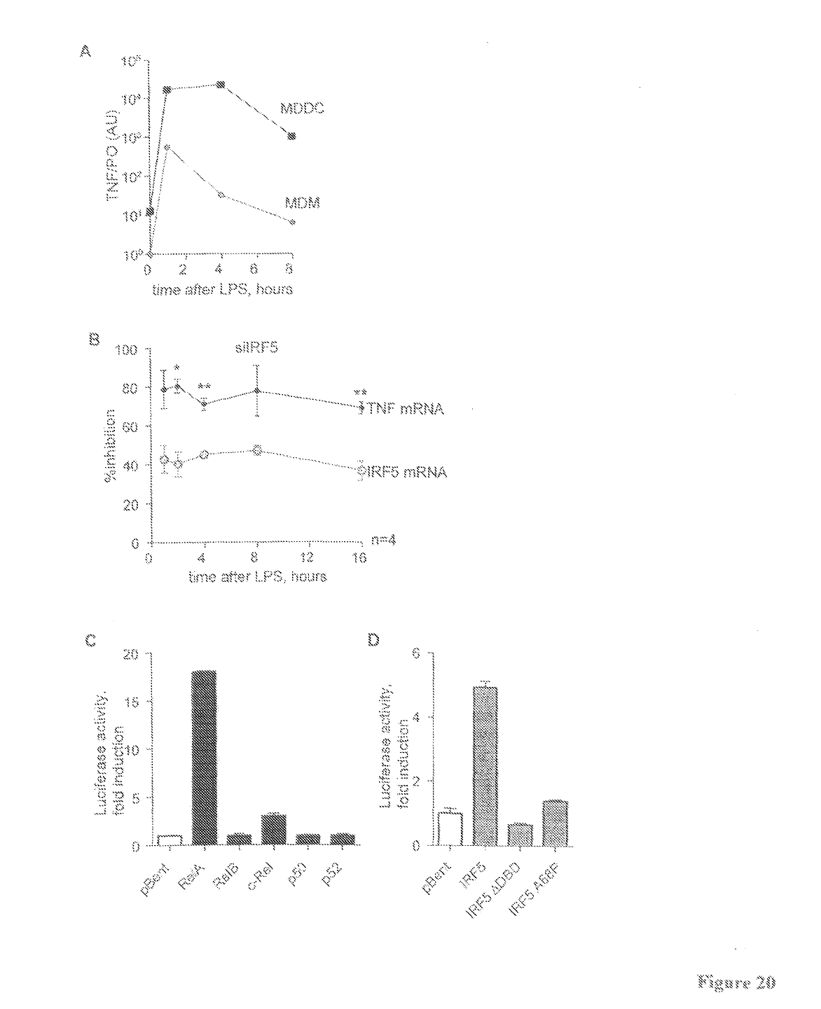

FIG. 21: IRF5 is recruited to the 5′ upstream and 3′ downstream region of TNF

(A) Schematic of the TNF locus. Protein coding and non-coding exons are shown in black and white. Putative ISREs and are allocated as white ovals; κB sites—as black circles. The approximate amplicon size of primer sets spanning the TNF locus (A to L) are indicated by black lines. CO—a control primer set containing neither an ISRE nor a κB site.

(B, C) HEK-293-TLR4-Md2/CD14 cells were left unstimulated or stimulated with LPS (1 μg/ml) for 4 and 24 h and analysed by ChIP with antibodies specific to IRF5 (B) or RelA (C).

(D, E) MDDCs were left unstimulated or stimulated with LPS for 1 h and 4 h followed by ChIP with antibodies specific to IRF5 (D) or RelA (E).

(F) Co-recruitment of RelA and IRF5 to region H was assessed by re-ChIP analysis with antibodies against RelA followed by IRF5-specific antibodies.

(B-F) Data show mean % input relative to genomic DNA (gDNA)±SD of a representative experiment. -AB—a no antibody control.

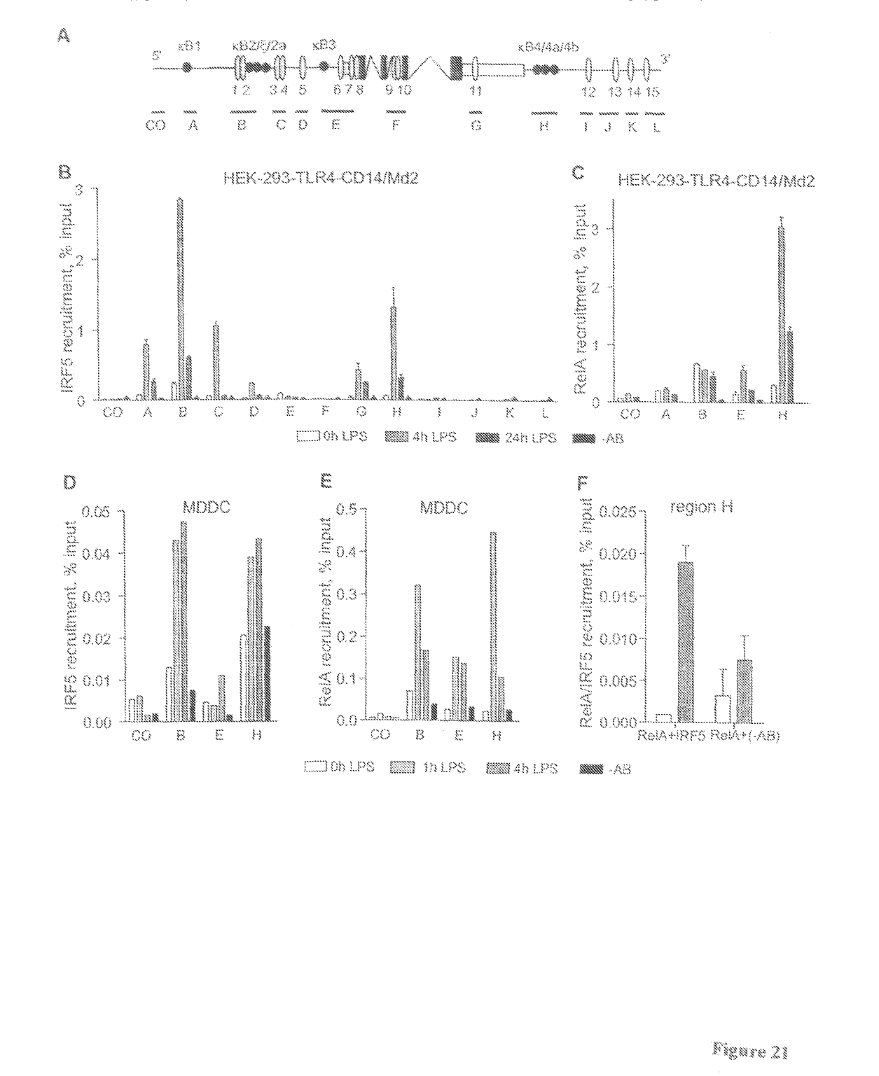

FIG. 22: Location of ISRE and kB sites in the TNF locus relative to transcription start site (TSS) and to ChIP amplicons used in the locus mapping.

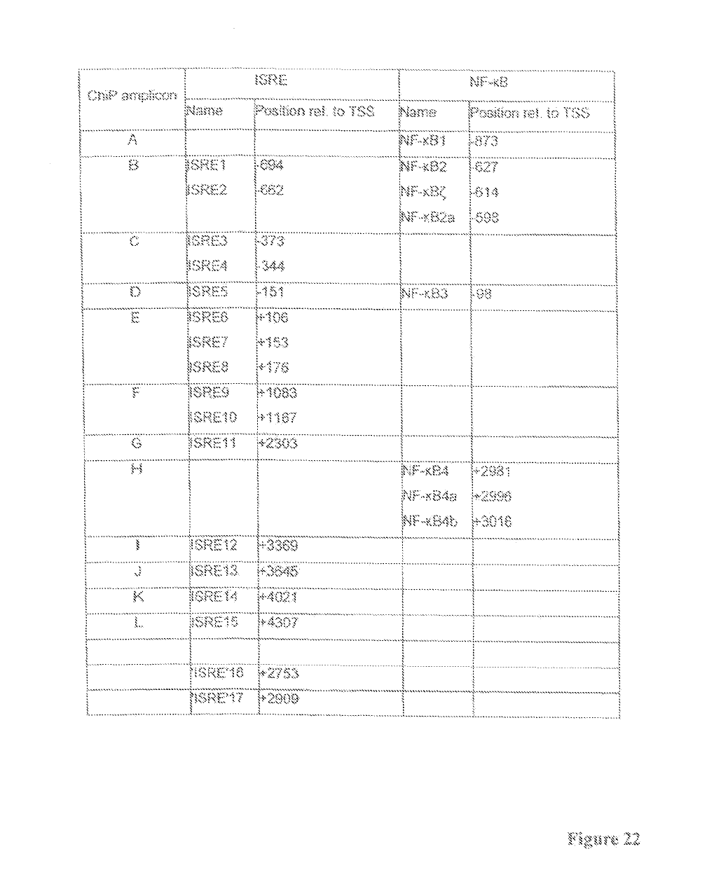

FIG. 23: LPS-induced recruitment of IRF5 and RelA to the TNF locus in MDDCs

(A) Recombinant purified IRF5-DBD was used in an EMSA with radioactive probe corresponding to the selected ISRE and κB sites. PRDI-III (IFN-˜ promoter) was used as a positive control.

(B, C) IRF5 and RelA are recruited to the 5′ upstream region Band 3′ downstream region H of the TNF gene. MDDCs were left untreated or stimulated with LPS for 1 h and 4 h followed by ChIP with antibodies specific to IRF5 (B) or RelA (C).

(D) LPS induces a rapid transcription of nascent TNF RNA. MDDCs were left untreated or stimulated with LPS for 0.5 h, 2 h and 4 h followed by ChIP with antibodies specific to Pol II and primers in the 3′ downstream of the TNF gene. (B-D) Data are shown as the mean±SEM of 5 (IRF5), 4 (RelA) or 3 (Pol II) independent experiments each using MDDCs derived from a different donor: *p<0.05, ** p<0.01 (One-way ANaYA).

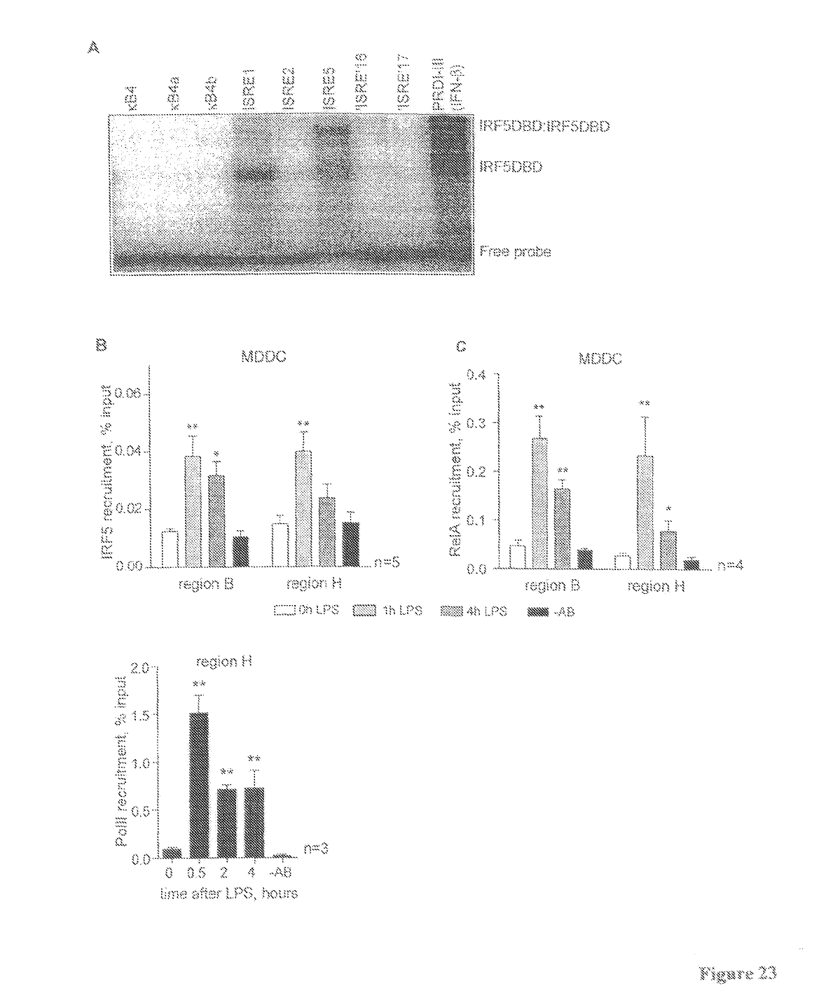

FIG. 24: IRF5 specifically interacts with RelA

(A) HEK-293-TLR4-Md2/CD14 cells were transfected with human IRF5 tagged with onestrep tag (N-terminus) and HA tag (C-terminus) (lanes 1,3) or an empty vector pBent (lanes 2,4) and fixed with formaldehyde. Crosslinks were reversed by heating and immunoblotted for bait IRF5 (anti-HA antibodies), or NF-κB subunits and tubulin.

(B) HEK-293-TLR4-Md2/CD14 cells were transfected with RelA-FLAG (lane 1) or BAP-FLAG (lane 2) Cells lysates were immunoprecipated with M2 anti-FLAG sepharose and immunobloted for bait RelA (anti-FLAG antibodies) or IRF5.

(C) MDDCs were stimulated with LPS for 1 h or left untreated. The endogenous interaction between RelA and IRF5 was examined by immunoprecipitation (IP) with anti-IRF5 antibody and immunoblotting with anti-RelA antibody. -AB—a mock IP.

(D) Nuclear pellet from triton extracted HEK-293-TLR4-Md2/CD14 cells was solubilised with DNasel and endogenous interaction between RelA and IRF5 was examined after IP as in (C).

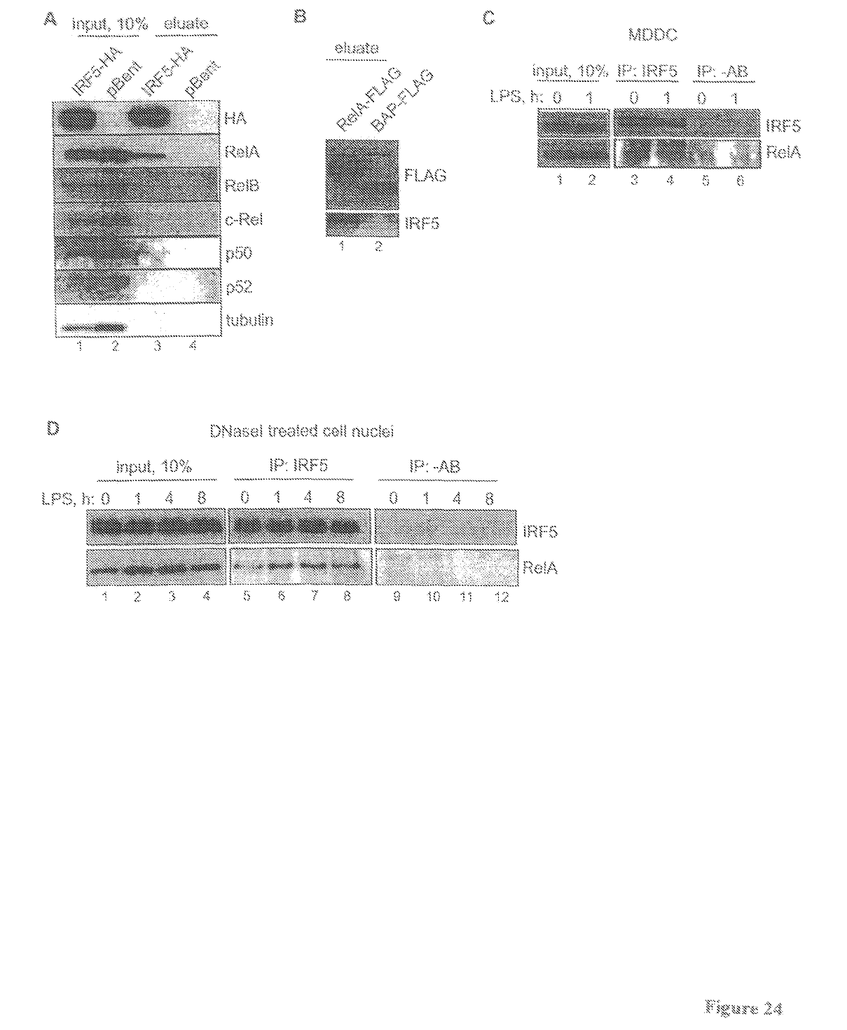

FIG. 25: RelA is required for IRF5-mediated activation of TNF

(A-C) HEK-293-TLR4-Md2/CD14 cells were transfected with siRNA against RelA (siRelA) or with non-targeting siRNA (siC) and used in ChIP analysis of RelA and IRF5 recruitment. Data indicate mean % input relative to gDNA±SD of a representative experiment. -AB—a no antibody control.

(A) 75% of RelA protein was degraded estimated by serial dilutions of the siC control sample analysed by Western blotting.

(B) Reduction in LPS-induced RelA recruitment to region H in siRelA treated cells.

(C) Reduction in LPS-induced IRF5 recruitment to region H in siRelA treated cells.

(D) HEK-293-TLR4-Md2/CD14 cells were transfected with the RelA, IRF5 and MyD88 expression constructs together with the TNF 5′ upstream/luciferase/TNF 3′ downstream reporter plasmids: 5′ wt/3′ wt—wild type construct, 5′ mut/3′ wt—mutated κBζ (GTGAATTCCC (SEQ ID No: 10)->tTGAATTCCC (SEQ ID No: 11)), κBζ (GTGATTTCAC (SEQ ID No: 12)->aTccTTTCAC (SEQ ID No: 13)), and κB2a (GGGCTGTCCC (SEQ ID No: 14)->taGCTGTGCCC (SEQ ID No: 15)) sites in the TNF 5′ upstream; 5′ wt/3′ mu—mutated κB4 (GGGAATTTCC (SEQ ID No: 16)->cGcAATgTgC (SEQ ID No: 17)) and κB4a (GGGAATTCCA (SEQ ID No: 18)->cGcAAgTgCA (SEQ ID No: 19)) sites in the TNF 3′ downstream; 5′ mut/3′ mut—all κB sites mutated. Data show means±SD and are a representative of 3 independent experiments, each performed in triplicate.

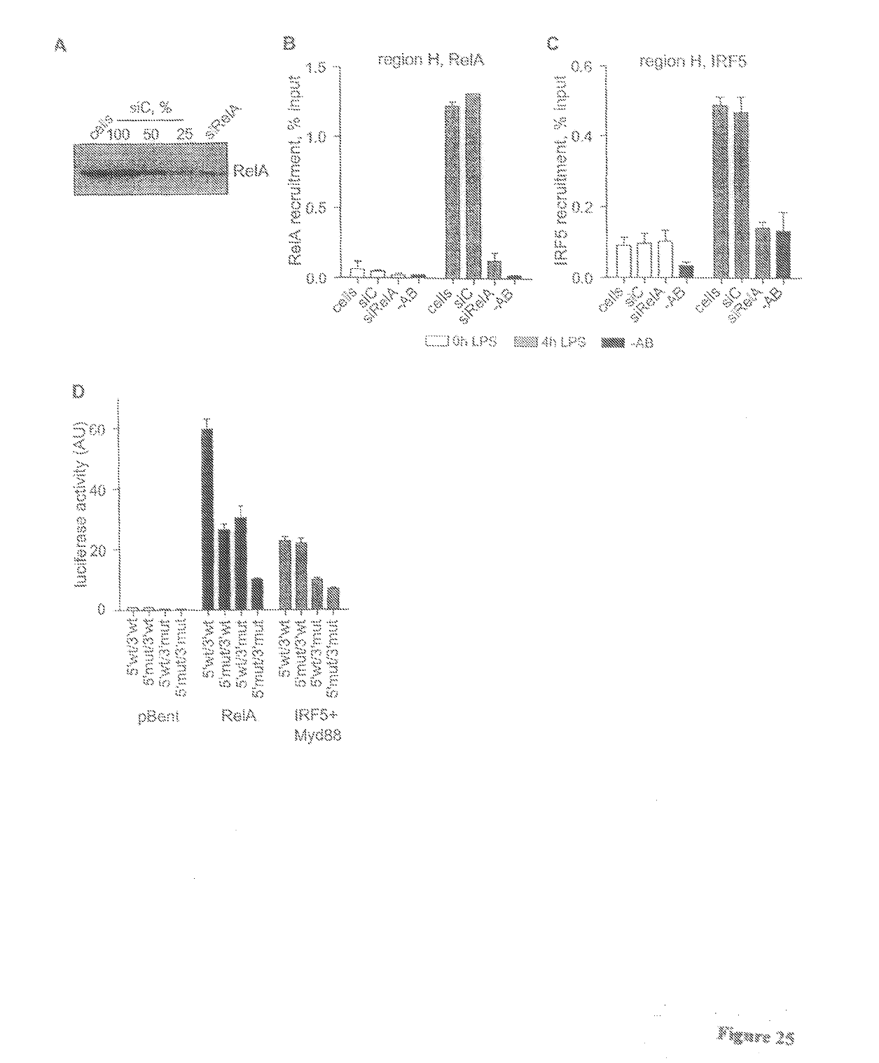

FIG. 26: IRF5 recruitment to the TNF 5′ upstream region is partly dependent on RelA

HEK-293-TLR4-Md2/CDI4 cells were transfected with siRNA against RelA (siRelA) or with non-targeting siRNA (siC) and used in ChIP analysis of RelA and IRF5 recruitment. Minimal reduction in LPS-induced IRF5 recruitment to the 5′ upstream region B in siRelA treated cells. Data indicate mean % input relative to gDNA±SD of a representative experiment.

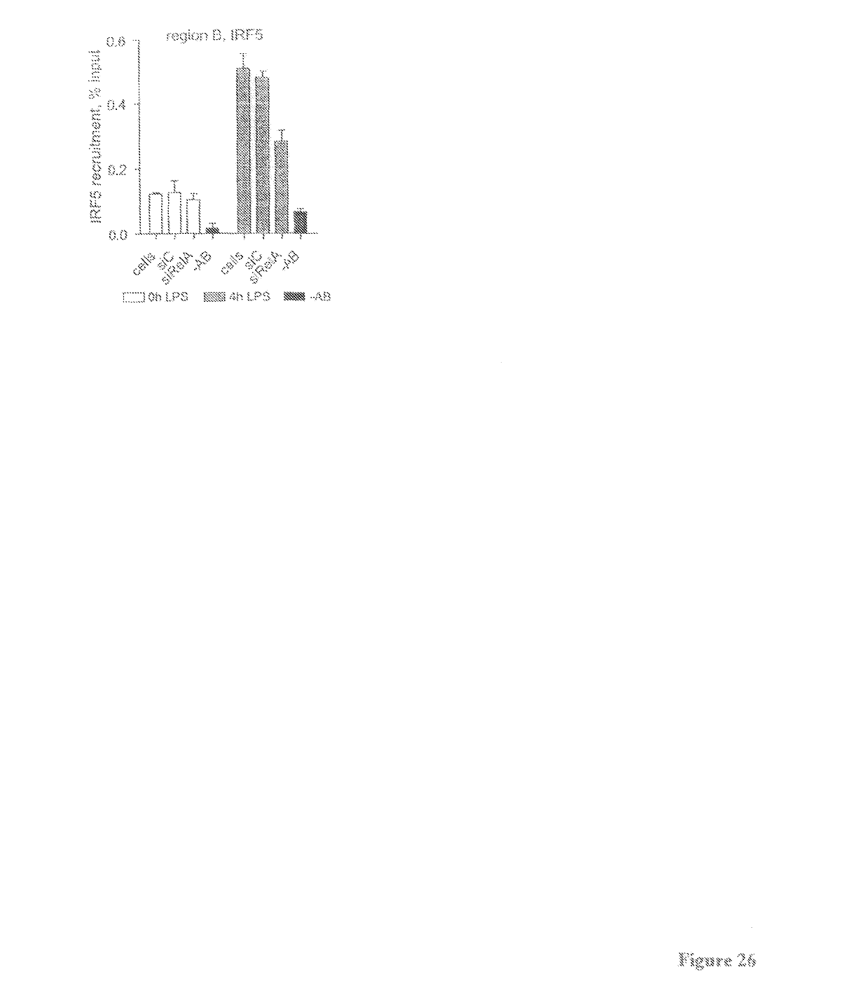

FIG. 27: NF-κB and IRF factors in human myeloid cells

(A) Monocytes, MDMs and MDDCs were either left untreated or simulated with LPS for 24 h and total protein extracts were subjected to western blot analysis. Actin was used as loading control. Representative blots of 4 independent experiments, each using cells from a different donor. The image is a composite made of two fragments of the same gel linked together.

(B) IRF5 can be detected in the nucleus of resting MDDCs. Western blot analysis of nuclear fractions of MDDCs upon stimulation with LPS (10 ng/ml) with antibodies against IRF5 or IRF3. Actin was used as a loading control.

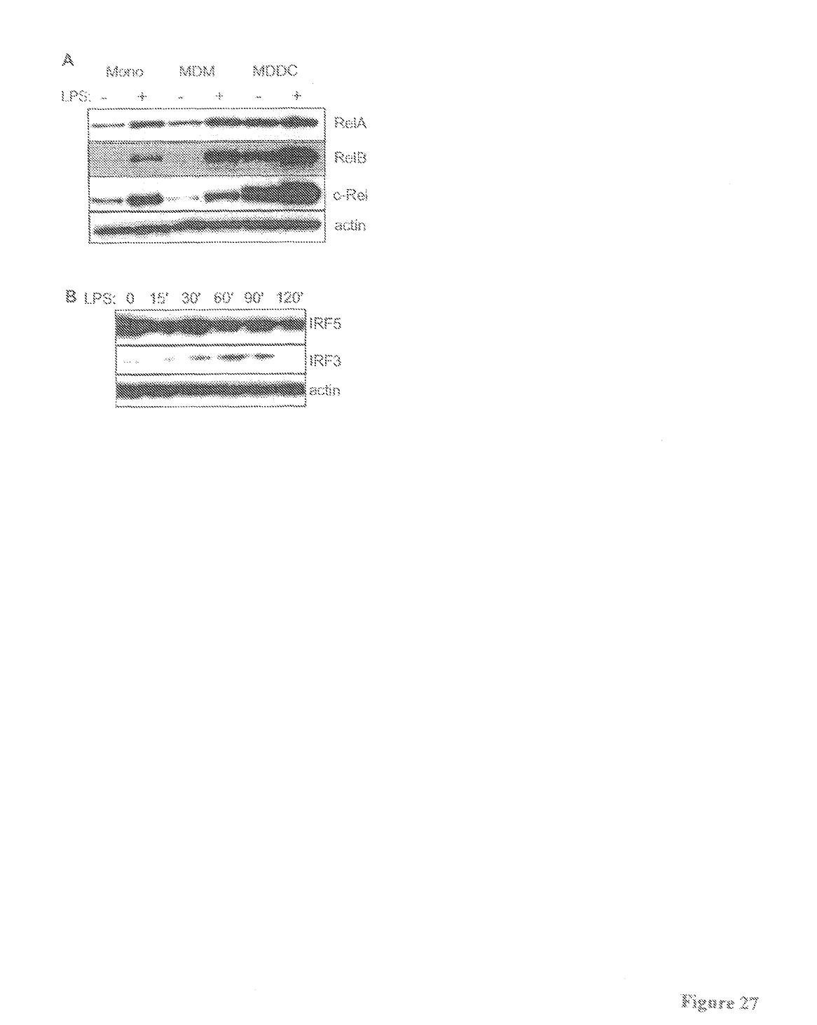

FIG. 28: Accumulation of Pol II at the transcription start side of TNF

MDDCs were left untreated or stimulated with LPS for 0.5 h, 2 h and 4 h followed by ChIP analysis with antibodies specific to Pol II. Data are shown as the mean±SEM of 3 independent experiments each using MDDCs derived from a different donor: *p<0.05, ** p<0.01 (One-way ANOY A).

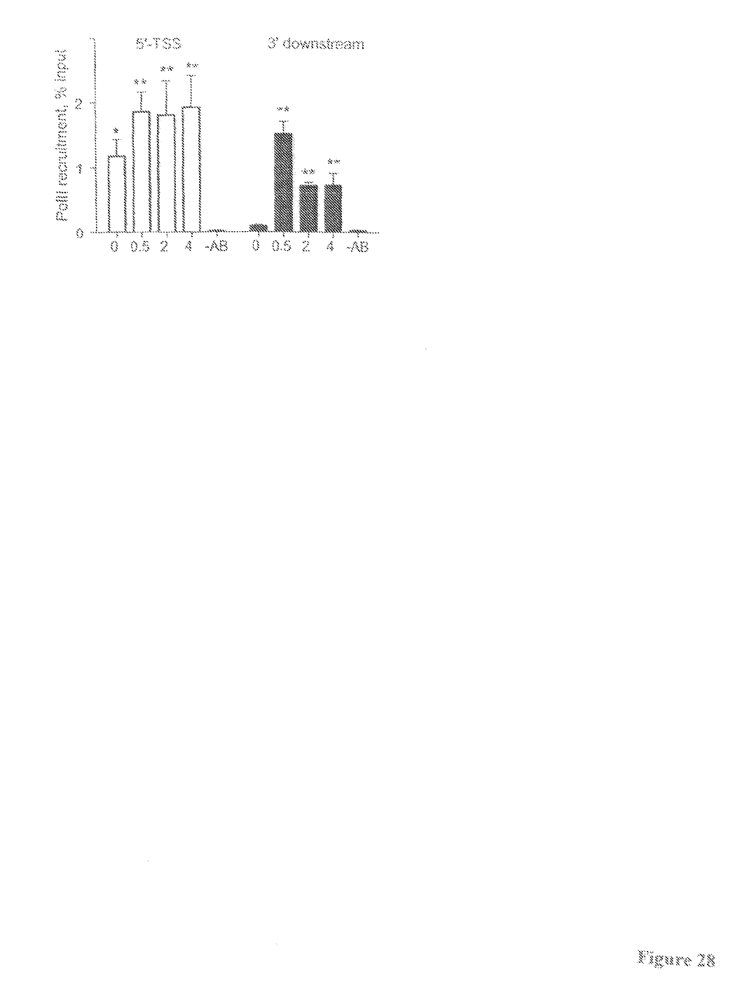

FIG. 29: A model for IRF5-RelA mediated induction of TNF in myeloid cells

LPS-induced recruitment of NF-κB RelA-containing complexes (grey ovals) to the 5′ upstream and 3′ downstream regions leads to transient TNF expression in MDMs. The mechanisms of transmitting activating signal from 3′ downstream to Pol II requires further investigation. IRF5 binding to DNA at the 5′ upstream and to RelA at the 3′ downstream establishes the possibility for region circularisation and recycling of Pol II molecules leading to sustained TNF expression in MDDCs.

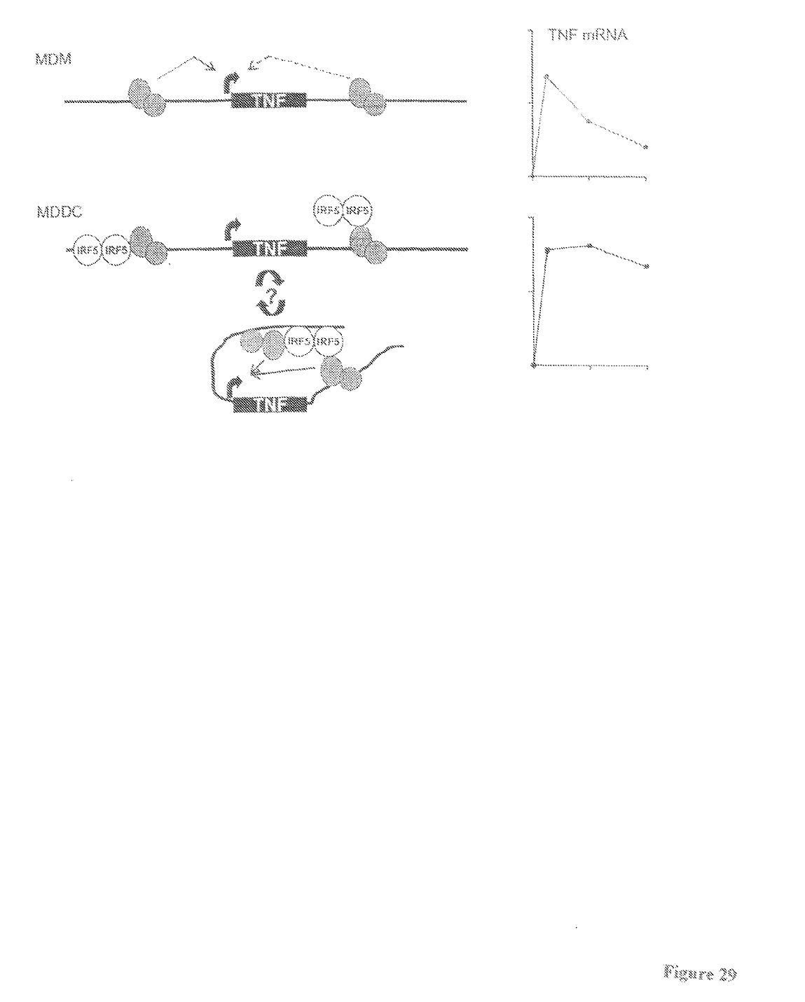

FIG. 30: is a schematic of One-Strep Protein:Protein Interaction Analysis

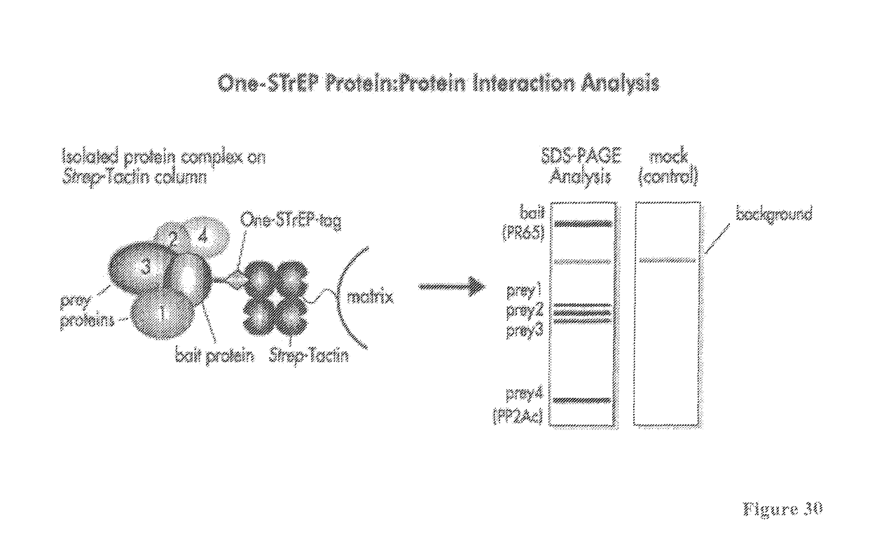

FIG. 31: Truncated mutants of IRF5 were cloned fused to the One-Strep tag at the N-terminus

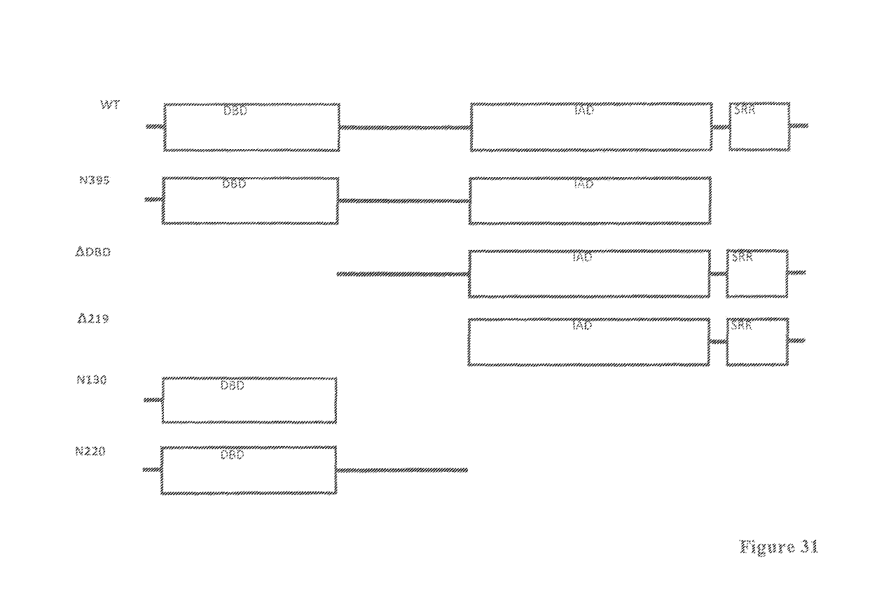

FIG. 32: Results obtained from the One-Strep analysis of the IRF5-RELA Interaction Interface.

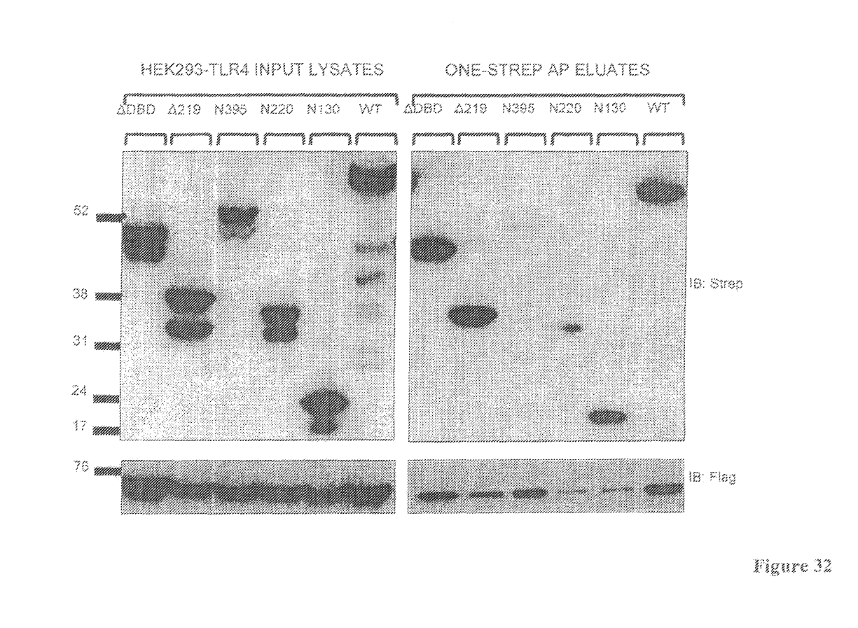

FIG. 33: Rel A dimerisation domain interacts with IRF5.

FIG. 34: Human IRF5 sequences.

(A) The amino acid sequence of human IRF5v3/4 (SEQ ID No: 1).

(B) The cDNA sequence of human IRF5 isoforms 3 and 4 (SEQ ID No: 2).

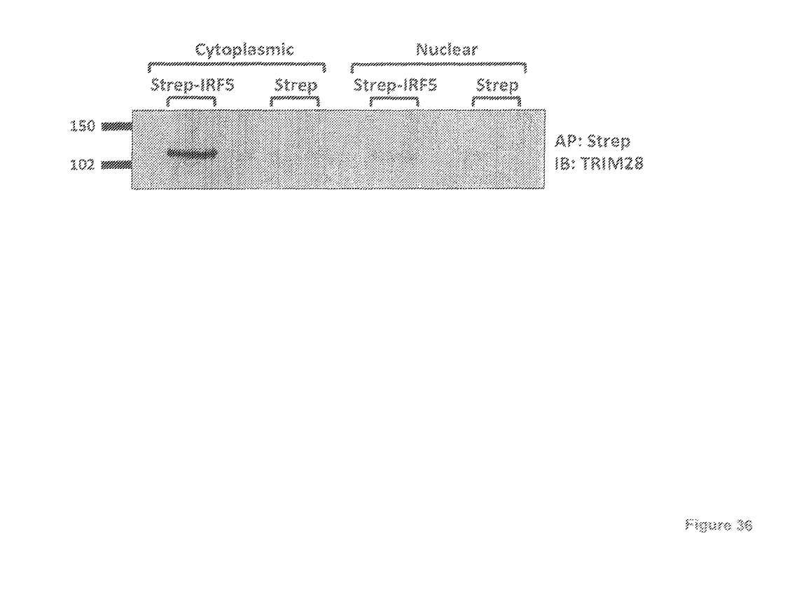

FIG. 345: SDS-PAGE gel showing IRF5 protein-protein interactions.

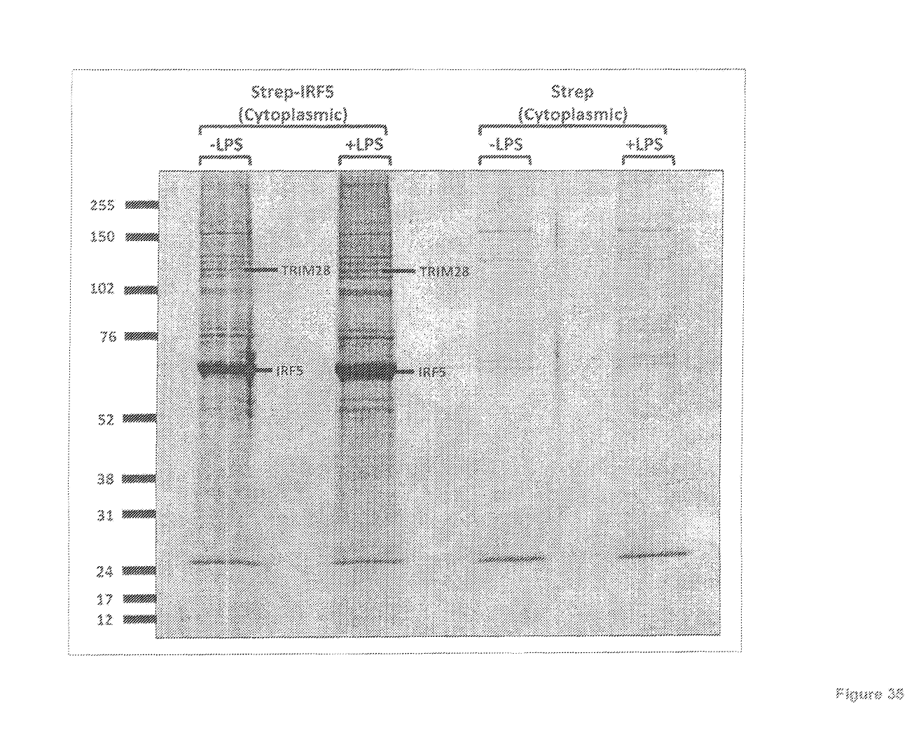

FIG. 36: Western blot confirming TRIM28 as an IRF5 interacting protein.

### EXAMPLE 1

### IRF5 Promotes Inflammatory Macrophage Polarization And TH1/TH17 Response

The information in Example 1 has been published by the inventors as Krausgruber et al “IRF5 promotes inflammatory macrophage polarization and T_{H}1-T_{H}17 responses”. (2011) Nature Immunology 12(3): 231-6, incorporated herein by reference.

### SUMMARY

Genetic polymorphisms in the interferon regulatory factor 5 (IRF5) gene, leading to increased IRF5 mRNA expression, are associated with a number of autoimmune diseases, including systemic lupus erythematosus and rheumatoid arthritis. Here we show that expression of IRF5 in macrophages is reversibly induced by inflammatory stimuli and contributes to plasticity of macrophage polarization. High levels of IRF5 are characteristic of pro-inflammatory M1 (IL-12^{high}, IL-23^{high}, IL-10^{low}) macrophages, in which it directly activates transcription of IL-12p40/p35 and IL-23p19 and represses IL-10 genes. Consequently, these macrophages set up the environment for a potent Th1/Th17 response. Global gene expression analysis demonstrates that exogenous IRF5 up- or down-regulates expression of established human markers of M1 or M2 (IL-12^{low}, IL-23^{low}, IL-10^{high}) macrophages respectively. Together our data suggest a critical role for IRF5 in M1 macrophage polarization and defines a novel function for IRF5 as a transcriptional repressor of IL-10.

### Introduction

Macrophages are a heterogeneous population of immune cells that are essential for the initiation and resolution of pathogen- or tissue damage-induced inflammation¹. They demonstrate remarkable plasticity that allows them to efficiently respond to environmental signals and change their phenotype and physiology in response to cytokines and microbial signals². These changes can give rise to populations of cells with distinct functions, which are phenotypically characterised by production of pro-inflammatory and anti-inflammatory cytokines³. Based on the Th1/Th2 polarization concept⁴ these cells are now referred to as M1 (classic) macrophages, that produce pro-inflammatory cytokines and mediate resistance to pathogens and tissue destruction, and M2 (alternative) macrophages, that produce anti-inflammatory cytokines and promote tissue repair and remodelling as well as tumour progression^{3, 5}.

The activation of a subset defining transcription factor (TF) is characteristic of a particular T cell lineage commitment: T-bet is associated with Th1, GATA3 with Th2, FOXP3 with Treg and RORγT with Th17 cells⁶. Dendritic cells (DCs) also employ subset-selective expression of IRF4 and IRF8 for their commitment. IRF4 is expressed at high levels in CD4⁺ DCs but low in pDCs. As a consequence, the CD4⁺ DC population is absent in irf4^{-/-} mice. Conversely, IRF8 is expressed at high levels in pDCs and CD8⁺ DCs, thus irf8^{-/-} mice are largely devoid of these DC subsets⁷. However, transcription factors underlying macrophage polarization remain largely undefined. Activation of NF-κB p50 has been previously associated with inhibition of M1 polarizing genes⁸, whereas CREB mediated induction of C/EBPβ has been shown to upregulate M2-specific genes⁹. More recent evidence suggests that, in mice, IRF4 may control M2 macrophage polarization by stimulating the expression of selected M2 macrophage markers¹⁰.

IRF5, another member of the IRF family, has diverse activities, such as activation of type I IFN genes, inflammatory cytokines, including TNF, IL-6, IL-12 and IL-23, and tumour suppressors¹¹. Consequently, IRF5 deficient mice are resistant to lethal endotoxic shock¹². Human IRF5 is expressed in multiple splice variants with distinct cell type-specific expression, cellular localization, differential regulation and functions¹³. Moreover, genetic polymorphisms in the IRF5 gene, leading to expression of several unique isoforms or increased expression of IRF5 mRNA, is implicated in autoimmune diseases including systemic lupus erythematosus (SLE), rheumatoid arthritis (RA), Sjogren's syndrome, multiple sclerosis and inflammatory bowel disease¹⁴⁻¹⁸. Here we show a role for IRF5 in determining M1 macrophage lineage commitment. M1 macrophages are characterised by high level of IRF5, expression of which is induced during their differentiation with either GM-CSF or IFN-γ/LPS. Forced expression of IRF5 in M2 macrophages drives global expression of M1-specific cytokines, chemokines and co-stimulatory molecules and leads to a potent Th1/Th17 response. Conversely, the induction of IL-12, IL-23, IL-16, TNF is impaired in human M1 macrophages with levels of IRF5 expression reduced by siRNA knock-down or in the peritoneal macrophages of the Irf5−/− mice. We provide the first insights into the molecular mechanisms behind its direct transcriptional activation of IL-12p40, IL-12p35 and IL-23p19 genes. We also discover a new function of IRF5 as a transcriptional inhibitor of IL-10 and other selected M2-specific molecules. Our data suggest that activation of IRF5 expression defines macrophage lineage commitment by driving M1 macrophage polarization. Our data, together with the results of Satoh et al demonstrating a role for IRF4 in controlling M2 macrophage markers¹⁰, establish a new paradigm for macrophage polarization and highlight the potential for therapeutic interventions via modulation of the IRF5-IRF4 balance.

### Materials and Methods

### Plasmids

Expression constructs encoding full length human IRF3, IRF5v3/v4, and IRF5ΔDBD were described in²². The vectors encoding IRF5 and IRF3 expressed similar levels of proteins, but only IRF5 resulted in a significant increase in TNF secretion, while only IRF3 induced type III IFNs²². IL-10 promoter driven luciferase-reporter constructs were previously described²⁸. The IL-12p35 wild-type and IL-12p35 ISRE mutant promoter constructs⁵⁰ were a kind gift from Prof Xiaojing Ma (Cornell University, USA). The sequences and restriction maps are available upon request.

### Mice

The generation of IRF5^{-/-} mice has been described¹². For generation of BMDMs/GM-CSF, bone marrow of wild-type or IRF5^{-/-} was cultured in RPMI (PAA, USA) supplemented with 50 ng/ml recombinant mouse GM-CSF (Preprotech, UK). After 8 days, adherent cells were washed with PBS, re-plated and stimulated with 100 ng/ml LPS (Alexis Biochemicals, USA). For in vivo experiment, littermate wild-type and IRF5^{-/-} mice were intraperitoneally injected with 20 ug LPS in 200 ul sterile PBS. Mice were sacrificed after 3 h and serum was collected. Spleens were removed and cultured in DMEM supplemented with 10 ng/ml anti-CD3 antibodies (BD Bioscience, USA) for 48 h.

### Cell Culture

Enriched populations of human monocytes were obtained from the blood of healthy donors by elutriation as described previously²². M1 and M2 macrophages were obtained after 5 days of culturing human monocytes in RPMI 1640 (PAA, USA) supplemented with 50 ng/ml GM-CSF or 100 ng/ml M-CSF (Peprotech, UK). Cells were stimulated with 10 ng/ml LPS (Alexis Biochemicals, USA) or 10 ng/ml LPS plus 50 ug/ml IFN-γ (Peprotech, UK). For “priming” experiments M1 macrophages at day 5 were simulated for 24 h with M-CSF (100 ng/ml). Similarly, M2 macrophages at day 5 were stimulated for 24 h with GM-CSF (50 ng/ml); IFN-γ (50 ng/ml) or LPS (10 ng/ml) plus IFN-γ (50 ng/ml).

### Measurement of Cytokine Production

Cytokine secretion was quantified with specific ELISAs for human IL-12p40, IL-12p70, IL-10, IFN-γ, TNF, CXCL10, IL-1β (BD Bioscience, USA); IL-23, CCL2 (eBioscience); CCL5, CCL13, CCL22 (R&D Systems) and IL-17A, IL-4 (Insight Biotechnology). Mouse cytokine secretion was quantified with specific ELISAs for Il-12p70, Il-23 and Il-10 (eBioscience); Ifn-γ, Il-17a (BD Bioscience, USA) and serum levels of mouse Il-1β, TNF, Il-6 and Il-10 were determined by BD™ cytometric bead assay (BD Bioscience, USA) on a FACS Canto II (BD Bioscience).

### Mixed Lymphocyte Reaction

Human macrophages were plated in 96-well plate at 2×10⁴ cells/well. T lymphocytes were isolated from the blood of healthy donors by elutriation, analysed by FACS and used if purity was >90%. T lymphocytes were added to macrophages at 5×10⁵ cells/well. Control cultures contained medium, T lymphocytes or M2 macrophages alone. After 72-96 h of co-culture supernatants were collected for detection of cytokines. For proliferation experiments, cells were pulsed with 1 μCi of [3H]thymidine (Amersham Biosciences, USA) 16 h before harvest and DNA synthesis was measured by [3H]thymidine incorporation using a Beckman beta scintillation counter (Beckman Instruments, USA).

### RNA Interference

siRNA-mediated knockdown was performed using On-target plus SMART pool reagent (Dharmacon, USA, catalogue No. L-011706-00-0005) designed to target human IRF5. DharmaFECT I® (Dharmacon, USA) was employed as the siRNAs transfection reagent according to manufacturers' instructions.

### Adenoviral Infection

Infections of M2 macrophages were performed as described previously²².

### RNA Extraction and Quantitative Real-Time RT-PCR

Total RNA was extracted from cells using a QiaAmp RNeasy mini kit (Qiagen, Germany) according to manufacturer's instructions. cDNA was synthesised from total RNA using SuperScript® III Reverse Transcriptase (Invitrogen, USA) and 18-mer oligo dTs (Eurofins MWG Operon, UK). The gene expression was analysed by ΔΔCt method based on the quantitative real-time PCR with TaqMan primer sets for human IL-12p35, IL-12p40, IL-23p19, IL-10, IFN-γ, IL-17A/F, IL-21, IL-22, IL-26, IL-23R, TBX21 (for T-bet), Mrc1, Arg1, Rentla (for Fizz1) and PO (Applied Biosystems) in an ABI 7900HT machine (Applied Biosystems, USA). RORγt was detected by SybrGreen with the primer set for the human RORC2 gene (RORC2_F1: TGAGAAGGACAGGGAGCCAA (SEQ ID No: 20); RORC2_R1: CCACAGATTTTGCAAGGGATCA (SEQ ID No: 21)).

### Luciferase Gene Reporter Assay

Infections of M2 macrophages were performed in 96-well plates in triplicate at a multiplicity of infection of 50:1. Cells were seeded in serum-free, antibiotics-free RPMI containing the desired number of viral particles in a final volume of 50 μl. Cells were infected with expression constructs coding for IRF5, IRF5ΔDBD or empty vector and after 6 h followed by infection with IL-10 luciferase constructs. Cells were allowed to recover for 24 hours before experimental assay. Co-transfections of HEK-293-TLR4/MD2 cells with the IL-12p35 wild-type and IL-12p35 ISRE mutant constructs were performed as described previously²².

### Total Protein Extracts and Western

Total protein extracts were prepared as previously described²². Equal amounts of proteins were resolved by SDS-PAGE and analysed with antibodies against IRF5 (ab2932 or ab21689, Abcam, UK), IRF3 (sc-9082×), IRF4 (sc-28696), p50 (sc-114x), RelA (sc-372x), all form Santa Cruz, USA, and actin (A5541, Sigma, USA).

### Flow Cytometry

For surface staining of T cells, cells were stained for 30 min at 4° C. with anti-CD4-FITC and anti-CD8-Per-CP-Cy5 (BD Bioscience). For intracellular cytokine staining (ICC), cells were stimulated for 3-4-h with phorbol myristate acetate (PMA), ionomycine and Brefeldin A (Sigma-Aldrich). Cells were stained for cell surface markers, fixed in Cytofix (BD Bioscience) and permeabilized using PBS containing 1% FCS, 0.01% sodium azide, and 0.05% saponin and stained with anti-IFN-γ-PB and anti-IL-17-PE (eBioscience). For surface staining, macrophages were incubated for 30 at 4° C. with anti-CD40-APC (eBioscience) and anti-CD163-PE (R&D Systems). Samples were run on a FACS Canto II (BD Bioscience) and analysed using FlowJo software (TreeStar).

### Chromatin Immunoprecipitation

ChIP assays were carried out essentially as previously described²² using antibodies against IRF5 (ab2932, Abcam, UK), Pol II (sc-899, Santa Cruz, USA) or IgG control (PP64, Milipore, USA). The immuno-precipitated DNA fragments were then interrogated by real-time PCR using SYBR®Premix Ex Taq II™ master mix (Takara Bio, USA) and the following primers:

IL12-p35 locus:
(SEQ ID No: 22)
(TCATTTTGGGCCGAGCTGGAG
and
(SEQ ID No: 23)
TACATCAGCTTCTCGGTGACACG);
IL-12p40 locus:
(SEQ ID No: 24)
(TCCAGTACCAGCAACAGCAGCAGA
and
(SEQ ID No: 25)
GTAGGGGCTTGGGAAGTGCTTACCTT);
IL-23p19 locus:
(SEQ ID No: 26)
(ACTGTGAGGCCTGAAATGGGGAGC
and
(SEQ ID No: 27)
ACTGGATGGTCCTGGTTTCATGGGAGA)
and
IL-10 locus:
(SEQ ID No: 28)
(CCTGTGCCGGGAAACCTTGATTGTGGC
and
(SEQ ID No: 29)
GTCAGGAGGACCAGGCAACAGAGCAGT).

Data were analysed using an ABI 7900HT software (Applied Biosystems, USA).

### Microarray Data Analysis

Gene expression data were obtained by hybridising a total of 24 samples from 6 experimental groups (n=4 per group) to IIlumina HumanHT-12 Expression BeadChips.

Raw data were exported from the IIlumina GenomeStudio software (v1.0.6) for further processing and analysis using R statistical software (Team, 2010) (v2.10) and BioConductor packages. Raw signal intensities were background corrected using array-specific measures of background intensity based on negative control probes, prior to being transformed and normalised using the ‘vsn’ package (Huber et al, 2002). Quality control analyses did not reveal any outlier samples. The dataset was then filtered to remove probes not detected (detection score <0.95) in any of the samples, resulting in a final dataset of 25,620 probes. Statistical analysis was performed using the Linear Models for Microarray Analysis (limma) package (Smyth et al, 2005). Differential expression between the experimental groups was assessed by generating relevant contrasts corresponding to the relevant comparisons. Raw p-values were corrected for multiple testing using the false discovery rate controlling procedure of Benjamini and Hochberg (1995), adjusted p-values below 0.01 were considered significant. Significant probe lists were then annotated using the relevant annotation file (HumanHT-12_V3_{—}0_R2_{—}11283641_A) that was downloaded from the Illumina website (http://www.illumina.com) for further biological investigation.

### Bioinformatics and Statistical Analyses

Nucleotide sequences were inspected with transcription factor binding site searching software JASPAR (http://jaspar.cgb.ki.se/) (Vlieghe et al, 2006) and Genomatrix (http://www.genomatix.de/) for the presence of putative ISRE sites (as shown in Supplementary Table S2 of Krausgruber et al (2011), which is incorporated herein by reference). Statistical analysis was performed using One-way ANOVA with Dunnett's multiple comparison post test or Student's T-test where appropriate (*p<0.05, **p<0.01, ***p<0.001).

### Results

### IRF5 is Highly Expressed in Human M1 Macrophages

The M1 macrophage phenotype is induced by Interferon gamma (IFN-γ) followed by stimulation with bacterial products like lipopolysaccharide (LPS) or by treatment of monocytes with granulocyte-macrophage colony-stimulating factor (GM-SCF) (FIG. 1a and Ref¹⁹⁻²¹). We examined the levels of IRF5 expression in primary human monocytes or in monocyte derived macrophages and observed an increase in the population differentiated with GM-CSF (FIG. 2a, FIG. 1b). Furthermore, treatment of monocytes with GM-CSF but not M-CSF resulted in upregulation of IRF5 mRNA expression within 4 h post stimulation (FIG. 2b). To account for possible differences in macrophage in vitro differentiation protocols, we analysed the level of IRF5 in macrophages treated with either IFN-γ alone or in combination with LPS for 24 h and found that these were similar to the ones in GM-CSF treated cells (FIG. 1d). The expression of IRF4, shown while this manuscript was under submission to control polarisation towards M2 phenotype 10, was equally induced during monocyte differentiation into M1 or M2 macrophages (FIG. 2a). The expression of IRF3, another member of the IRF family central to the innate immune response, was not affected by differentiation into macrophage subtypes (FIG. 2a).

Thus, we concluded that IRF5 is induced in pro-inflammatory M1 macrophages independently of the in vitro differentiation protocol, whereas the levels of IRF4 and IRF3 are comparable between the macrophage populations. Of interest, we observed no significant difference between M1/M2 macrophages in the basal or LPS-induced levels of NF-κB p50 protein, previously implicated in macrophage polarisation towards M2 phenotype⁸ (FIGS. 1c,e).

### IRF5 Expression is Plastic During Human Macrophage Polarization

To examine whether IRF5 may contribute to plasticity of macrophage polarisation, we attempted to convert one population into another by culturing M2 macrophages with GM-CSF and M1 macrophages with M-CSF. As expected, treatment of M2 macrophages with GM-CSF or IFN-γ led to production of M1 phenotypic markers upon LPS stimulation (M2->M1) (FIG. 3a). Conversely, treatment of M1 macrophages with M-CSF led to production of M2 phenotypic markers upon LPS stimulation (M1->M2) (FIG. 3b). Significantly, M2->M1 conversion of macrophages led to an increase in IRF5 protein levels (FIG. 2c, FIG. 3c), whereas M1->M2 macrophages had reduced levels of IRF5 (FIG. 2d, FIG. 3d). Once again, the levels of IRF4 or IRF3 appeared to be unchanged (FIGS. 2c, d).

These results demonstrate that expression of IRF5 is quickly adapted to the varying concentrations of environmental stimuli, suggesting that this factor may participate in establishing plasticity of macrophage polarization.

### IRF5 Influences the Production of Human Macrophage Lineage Specific Cytokines

We next investigated whether IRF5 would directly induce expression of M1 macrophage phenotypic markers. Bioactive IL-12p70 and IL-23 were detected in M2 macrophages infected with the adenoviral expression construct encoding for human IRF5 (variant 3/4)¹³, but minimal with IRF3 or an empty vector²² (FIG. 4a). The up-regulation of IL-12p70 and IL-23 was reflected by a vast increase in secretion of the p40 subunit shared by the two cytokines (FIG. 4a). The secretion of both IL-12p70 and IL-23 peaked at 24 h post LPS stimulation and remained sustained up to at least 48 h (FIG. 5a). We also observed a significant increase in production of other key pro-inflammatory cytokines such as IL-1 and TNF by IRF5-expressing macrophages (FIG. 5b). Remarkably, IL-10 production in the IRF5 expressing cells was noticeably reduced (FIG. 4a). We also observed IL-10 protein inhibition in cells over-expressing IRF3 (FIG. 4a), which may represent a negative feedback regulation of IL-10 expression²³, since the main direct target of IRF3, IFN-β, induces IL-10²⁴. The complementary experiment targeting endogenous IRF5 in M1 macrophages by RNA interference (RNAi) (FIG. 5c) resulted in significant inhibition of IL-12p70 and IL-23 and increase of IL-10 (FIG. 4b). Moreover, secretion of IL-12p40 was also reduced in these cells (FIG. 4b), consistent with the data obtained in mouse myeloid cells deficient in Irf5¹². Taken together, IRF5 influences M1 macrophage polarisation by equipping the cells with the IL-12^{high}, IL-23^{high}, IL-10^{low} cytokine profile.

### IRF5 Promotes Human Th1/Th17 Response

One of the hallmarks of M1 macrophage polarisation is acquired antigen presenting features leading to efficient Th1 response^{20, 21}. To examine whether IRF5-aided the polarisation of T lymphocyte proliferation, fate or activation state, human M2 macrophages were infected with IRF5, IRF3 expression constructs or an empty vector and exposed to human T lymphocytes extracted and purified from peripheral blood of major histocompatibility complex (MHC) unmatched donors in a mixed lymphocyte reaction (MLR). Total T lymphocyte proliferation was determined 3 days after infection by measuring thymidine incorporation, while activation of specific T cell subsets was analysed by Fluorescence Activated Cell Sorting (FACS) using appropriate antibodies (FIG. 6a). Proliferation of T lymphocytes (FIG. 6b) was considerably higher when co-cultured with IRF5 expressing macrophages. Furthermore, only IRF5 expressing macrophages provided the cytokine environment for Th1 expansion and activation, assessed by increased number of IFN-γ producing CD4⁺ cells (FIG. 7a and FIG. 6c) and mRNA (FIG. 8e) and protein (FIG. 7b) expression of IFN-\tilde{γ} In these cultures we also observed expansion and activation of Th17 cells, assessed by increased number of IL-17 producing CD4⁺cells (FIG. 7d and FIG. 6d), secretion of IL-17A (FIG. 7e) and mRNA expression of IL-17A/F, IL-21, IL-22, IL-26 and IL-23R (FIG. 6f). In line with recent studies demonstrating that IL-23 enhanced the emergence of IL-17+/IFN-γ+ population of T cells²⁵, about 25% of IL-17 positive cells were also positive for IFN-γ (data not shown), supporting a close developmental relationship of human Th17 and Th1 cells²⁶. mRNA expression of Th1/Th17 subset defining transcription factors, i.e. T-bet and RORyt was significantly induced in T cells co-cultured with IRF5 expressing macrophages (FIGS. 7c and 7f). Of interest, expression of GATA3 and FOXP3 mRNA was reduced in the presence of IRF5 expressing macrophages (data not shown). Hence, IRF5 promotes T lymphocyte proliferation and activation of the Th1/Th17 lineages, but does not induce Th2 or Treg lineages.

### IRF5 is Directly Involved in Transcriptional Regulation of Human Lineage Specific Cytokines

IRF5 is a transcription factor which can bind to the regulatory regions of target genes and modulate their expression. Here we determined whether the role of IRF5 in differential regulation of IL-12p70, IL-23 and IL-10 cytokine secretion was a direct consequence of its function as a transcription factor. mRNA expression of IL-12p40, IL-12p35 and IL-23p19 was strongly induced in M2 macrophages infected with adenoviral vector constructs encoding for IRF5, but not IRF3 or an empty vector (FIG. 8a). Moreover, the IRF5-driven IL-12p40 mRNA expression was sustained until at least 16 h post LPS stimulation (FIG. 9a). Consistent with the protein secretion data, expression of IL-10 mRNA was inhibited by IRF5 (FIG. 8a). However, expression of IL-10 mRNA was not altered by IRF3, suggesting the lack of a direct role for IRF3 in IL-10 transcription. RNAi-mediated inhibition of endogenous IRF5 in M1 macrophages reduced IL12p40, p35 and IL23p19 mRNA expression 8 h post LPS stimulation in cells from multiple blood donors (FIG. 8b). IL12p40 was strongly inhibited throughout the analysed time course, even 16 h post LPS stimulation (FIG. 9b). The expression of IL-10 mRNA was increased in the cells with knocked-down levels of IRF5 (FIG. 8b).

To formally define the global expression profile induced by IRF5, we carried out genome-wide expression analysis, in which M2 macrophages transduced with ectopic IRF5 were compared to previously defined human M1 and M2 macrophage subsets^{21, 27}. We found that expression of about 90% of known human polarization specific markers was driven by IRF5 (FIG. 8c). IRF5 induced 20 M1-specific and inhibited 19 M2-specific genes encoding cytokines, chemokines, co-stimulatory molecules and surface receptors (FIG. 8c) resulting in higher or lower production of corresponding proteins (FIGS. 9c, d). Moreover, we identified a number of novel IRF5-regulated genes that are likely to contribute to the main functional features of macrophage subsets, such as phagocytosis and antigen presentation (FIG. 10).

Next, we investigated the LPS-induced recruitment of IRF5 to the respective promoter loci. All IRF family members share a well-conserved N-terminal DNA binding domain (DBD) that recognises IFN-stimulated response elements (ISREs). A computational analysis of the regions −2000 nt 5′ upstream and +1000 nt downstream of the transcription start site (TSS) of IL-12p40/p35, IL-23p19, IL-10 and other IRF5-regulated genes (FIGS. 10 and 11) led to the identification of several ISREs (as shown in Supplementary Table S2 of Krausgruber et al (2011), which is incorporated herein by reference). Primers, encompassing these ISREs, were designed and used in quantitative ChIP experiments in M1 macrophages stimulated with LPS for 0, 1, 2, 4, 8, and 24 h. We observed LPS-induced enrichment of IRF5 to the IL-12p40/p35 and IL-23p19 promoter regions up to 8 h post stimulation, matching the kinetics of Pol II recruitment to the genes (FIGS. 11a-c). On the contrary, at the IL-10 promoter region LPS-induced IRF5 recruitment took place between 1 and 4 h post stimulation, whereas Pol II could bind to the region only 8 h post stimulation (FIG. 11d), suggesting a new inhibitory role for IRF5 in transcriptional regulation of selected genes.

Taken together, IRF5 regulates transcription of IL-12p40/p35, IL-23p19 and IL-10 genes via recruitment to their promoter regions. It also influences the expression of the majority of human lineage defining cytokines.

### IRF5 Inhibits Transcription of the Human IL-10 Gene

To investigate whether IRF5 can directly repress transcription of the IL-10 gene, we used an adenovirus construct with a gene-reporter in which the luciferase-reporter construct was flanked with 195 nt 5′ upstream of the IL-10 gene (IL-10-luc wt)²⁸. The IL-10-luc wt construct was co-infected with HA-tagged IRF5 or empty vector pENTR into M2 macrophages and luciferase activities were quantified. IRF5 expressing cells showed a significant decrease in luciferase activity in both un-stimulated and 4 h post LPS (FIG. 12a). To confirm the importance of IRF5 binding to the IL-10 promoter, we generated a mutant of IRF5 lacking the DNA binding domain (IRF5 ΔDBD). The IRF5 ΔDBD was no longer able to inhibit the IL-10 wt luciferase reporter (FIG. 12a). To further explore the molecular mechanism of IRF5-mediated suppression of IL-10 transcription, we introduced point mutations into the identified ISRE (−182/−172 nt relative to the TSS) and co-infected IL-10-luc ISRE mut construct it together with HA-tagged IRF5 and empty vector pENTR into M2 macrophages. The IL10-luc ISRE mut showed a different response to the wild type in that ectopic IRF5 was no longer able to suppress luciferase activity (FIG. 12b), suggesting that IRF5 is inhibiting IL-10 by direct binding to the IL-10 promoter ISRE. This is opposite to the positive regulatory activity of IRF5 at the TNF²² and IL-12p35 promoters (FIG. 13).

Here, we have shown the first evidence that IRF5 can act not only as a transcriptional activator, but also as a suppressor of selected target genes, in this case the anti-inflammatory mediator IL-10. The mode of inhibition is mediated by direct binding of IRF5 to the promoter region of IL-10 and likely engagement of yet to be identified novel co-factors.

### IRF5 Plays a Major Role in Mouse In Vivo Model of M1 Polarizing Inflammation

Similarly to their human counterparts, GM-CSF differentiated mouse bone marrow derived macrophages (GM-BMDMs) had higher levels of IRF5 expression compared to the M-CSF derived cells (M-BMDMs) (FIG. 14a) and were the only cells secreting IL-12p70 and IL-23 (data not shown). Consequently, GM-BMDMs from Irf5^{-/-} animals secreted significantly less IL-12p70, IL-23 or more IL-10 in response to LPS stimulation (FIG. 14b). No difference in IL-10 secretion was observed in M-BMDMs from wild type or Irf5−/− animals (data not shown).

To investigate the functional role of IRF5 in in vivo model of M1 polarizing inflammation, Irf5−/− mice were challenged with an intra peritoneal administration of LPS. Within 3 h of a sub-lethal dose of LPS injection, we observed a significant difference in the serum level of selected cytokines between wild type and Irf5−/− mice: consistent with the human data, secretion of IL-12p40, IL-23 (FIG. 14c), IL-1β, TNF, as well as IL-6 (FIG. 15), was reduced but of IL-10 was elevated (FIG. 14c). The animals injected with PBS secreted no cytokines. The number of macrophages recruited in the peritoneal cavity of LPS-challenged mice was similar in wild type and Irf5−/− animals (data not shown), but the expression of genes encoding M1 macrophage markers, i.e. Il-12p35/p40, Il-23p19, Il-1b, Tnf and Il-6, was significantly impaired in these cells (FIG. 14d). The expression of genes encoding M2 markers in Irf5−/− animals, i.e. Il-10, arginase 1 (Arg1), Fizz1 and Ym-1, was either significantly increased or showed a positive trend (FIG. 14d and data not shown). In addition, in splenocytes from the LPS-challenged Irf5−/− animals cultured ex vivo for additional 48 h we observed the noticeably affected levels of IFN-γ and IL-17 (FIG. 14e).

### Induction of IRF5 Expression in Experimental Models of Arthritis:

We used two mouse models of arthritis to assess the behaviour of IRF5 during their progression: (1) collagen induced arthritis (CIA), in which mice are intradermally immunised with type II collagen (CII) emulsified in complete Freund's adjuvant (CFA) and about 3 weeks later develop systemic, polyathritic disease, which affects multiple joints including those of the paw; (2) antigen-induced arthritis (AIA), in which mice develop a chronic mono-arthritic disease in the joint after subcutaneous immunization with methylated BSA (mBSA) antigen, followed 7d later by intra-articular injection of mBSA into one knee joint (Asquith et al (2009); Inglis et al (2007). Both models demonstrated a significant increase in the IRF5 mRNA expression with the arthritis flare (FIGS. 16A, B). Moreover, during AIA IRF5 deficient mice have impaired expression of TNF, a key mediator of inflammation in RA, and other M1 cytokines (IL-12, IL-23, IL-1β, IL-6), as well as IL-17 in their affected joints (FIG. 16C).

In summary, our data together with the previously reported role of IRF5 in LPS-induced lethal endotoxic shock¹², support a major role of IRF5 in establishing pro-inflammatory macrophage phenotype in animal models of M1 polarizing inflammation, e.g., arthritis.

### Discussion

Macrophages are key mediators of the immune response during inflammation. Plasticity and functional polarization are hallmarks of the macrophage system resulting in phenotypic diversity of macrophage lineage populations²⁹. Taking into account that the deficiency of IRF5 in mice leads to diminished production of IL-12p40 and IL-23p19^{11, 12}, universal markers of M1 macrophage subsets, we investigated whether IRF5 is involved in macrophage polarisation. We demonstrate that IRF5 is indeed a major factor defining macrophage lineage commitment: it is highly expressed in M1 macrophages and induces a characteristic gene expression and cytokine secretion profile, and promotes a robust Th1/Th17 response. We also unravel a new regulatory role for IRF5 as an inhibitor of M2 macrophage markers' expression. Finally, IRF5 contributes to the macrophage system plasticity, i.e. modulation of its levels leads to the conversion of one macrophage subset phenotype into the other.

The rapid and potent transcriptional response developed by macrophages encountering microbial stimuli, such as LPS, or subsequently cytokines, is orchestrated by many TFs. Among them are class III TFs, such as PU.1, C/EBPb, RUNX1, IRF8, which are lineage-specific transcriptional regulators turned on during macrophage differentiation³⁰. The combinatorial expression of these proteins specifies the macrophage phenotype via constitutive activation or repression of genes and chromatin remodelling at inducible loci. For instance, PU.1 is required for maintaining H3K4me1 enhancer marks at macrophage-specific enhancers³¹. But only a small proportion of the macrophage transcriptome is altered by cell polarization²⁷ and among the genes differentially expressed between the M1 and M2 subsets are those regulated by IRF5, such as IL-12p40, IL-12p35, IL-23p1β, TNF, macrophage inflammatory protein 1a, Rantes, CD1a, CD40, CD86, CCR7 (FIG. 11c). Another member of the IRF family, IRF4, known to inhibit IRF5 activation by competing for interaction with Myd88³², has been recently reported to control the expression of prototypical mouse M2 macrophage markers¹⁰. Of interest, we found that the expression of IRF4 is equally induced by M-CSF or GM-CSF differentiation (FIG. 2) and is further enhanced by exposure to IL-4³³. IRF5 expression, on the other hand, is specifically induced by GM-CSF or IFN-γ (FIGS. 1, 2), but is unresponsive to IL-4 (data not shown). Thus, IRF5 and IRF4 may be classified as class III TFs but with a caveat that they define specific macrophage subsets rather than the global macrophage lineage. NF-κB proteins, in particularly c-Rel and RelA, are important for expression of M1-specific cytokines^{34, 35}. We have recently shown that IRF5 and RelA cooperate in induction of the TNF gene²². It is interesting to speculate that the genes encoding IL-12, IL-23 subunits and other M1-specific markers might be under similar joint transcriptional control. Thereby, IRF5 may participate in the combinatorial assembly with macrophage-specific TFs, e.g. PU.1, and environmentally induced NF-κB³¹, to define the activity of specifically M1 enhancers.

The role of IRF5 in the inhibition of IL-10 gene transcription is novel and important in view of its well documented immunosuppressive activity. IL-10^{-/-} mice develop spontaneous autoimmune diseases and show increased resistance to infection³⁶. IL-10 represses immune responses by down-regulating inflammatory cytokines like TNF³⁷ and is important for generation of Treg cells, that act to suppress activation of the immune system and thereby maintain immune system homeostasis and tolerance to self-antigens³⁸. Major producers of IL-10 include M2 macrophages, B cells and T cells³⁹, whereas M1 macrophages and DCs are only weak producers²¹. Ectopic expression of IRF5 in M2 macrophages reduces IL-10 secretion upon LPS stimulation (FIG. 4d) and also affects mRNA expression of IL-10 and a number of other markers of human M2 macrophage phenotype, such as mannose receptor C type I, insulin-like growth factor 1, CCL2, CCL13, CD163, MCSF receptor and macrophage scavenger receptor 1 (FIG. 11c). Consistent with other studies⁴⁰ we find no expression of the most widely used prototypical mouse M2 markers (Arg1, Ym1, Fizz 1) in human macrophages (data not shown), while their expression in mouse LPS-elicited peritoneal macrophages showed a positive trend in the absence of IRF5 (FIG. 14). Of interest, expression of some chemokines, defined as M1 (CXCL10) or M2 (CCL17, CCL18, CCL22) markers in mouse macrophages did not follow the expected pattern of IRF5 dependence, i.e. induction for M1 and inhibition for M2 (see middle sector in FIGS. 8c and 9e), possibly reflecting on species-specific gene repertoire⁴¹. While human M1 but not M2 macrophages have been shown to secrete high levels of CCL22²¹, there is some controversy in the literature to whether CXCL10 is a marker of M1 or M2 macrophage phenotypes^{21, 24, 27}, our data agree more with the latter model.

The swift modulation of IRF5 expression and cytokine production by CSFs can help to explain the remarkable plasticity of macrophages in adjusting their phenotype in response to environmental signals². M-CSF is constitutively produced by several cell types, including fibroblasts, endothelial cells, stromal cells and osteoblasts. It is likely that this steady state production of M-CSF is polarising macrophages towards the M2 phenotype by keeping the IRF5 expression low (FIG. 1). By contrast, GM-CSF production by the same cell types requires stimulation and occurs usually at a site of inflammation or infection, which is also characterised by high levels of IFN-γ. Resolution of inflammation may once again coincide with predominance of M-CSF and switch in IRF5-driven cytokine production, as treatment of M2->M1 macrophages with M-CSF restores the original M2 phenotype (M2->M1->M2) (FIG. 3e). Activation of both the GM-CSF and IFN-γ receptors stimulates the Janus kinase-signal transducer and activator of transcription (JAK-STAT) pathway²⁹, and an ISRE element within the IRF5 promoter can bind STAT1/STAT2¹³, suggesting a possible mechanism for GM-CSF- and IFNγ-induced IRF5 expression. Consequently, high levels of IRF5 results in macrophage phenotype polarization towards M1 (FIGS. 2, 3). Significantly, IRF5 induces expression of IFN-γ mRNA (FIG. 10), pointing to an autocrine loop in macrophage polarization.

IRF5 expressing macrophages promote T lymphocyte proliferation and activation and drive them towards Th1 and Th17 phenotypes via secretion of IL-12⁴² and IL-23/IL-1β⁴³ respectively (FIG. 7). Th1 cells constitutively express IFN-γ and T-bet, Th17-RORγT, IL-23R, IL-17A/F, IL-21, IL-22 and IL-26. All these Th1/Th17 markers are up-regulated in the presence of IRF5-expressing macrophages (FIGS. 6e, f and 7). Human Th17 cells seem to exhibit different features from murine Th17 cells: while murine Th17 originate from a precursor common to Treg cells when IL-6 is produced in combination with TGF-β, human Th17 cells originate from CD161+CD4+ precursors in the presence of IL-23 and IL-1β, with little involvement of IL-6 and indirect role for TGF-β⁴³. Perhaps not surprisingly, dependence of IL-6 expression on IRF5 is much greater in mouse macrophages (FIGS. 8 and 14).

Both T cell subsets promote cellular immune function and have the capacity to cause inflammation and autoimmune diseases, such as inflammatory bowel disease and collagen-induce arthritis (CIA)^{44, 45}. Significantly, higher levels of Irf5 mRNA have been found in splenic cells from certain autoimmune-prone mouse strains than in non-autoimmune mice⁴⁶, while IRF5 deficient mice show impaired production of Th1/Th17 cytokines (FIG. 14). This points out towards a possible broad effect of therapies targeting the induction of IRF5 expression by macrophages, for example by targeting IRF5 inducing stimuli. Related to this, GM-CSF deficient mice fail to develop arthritis despite making a normal humoral immune response to the arthritogenic stimulus⁴⁷ and the blockade of GM-CSF in wild-type mice controls disease activity and levels of pro-inflammatory mediators in the joints⁴⁸.

In summary, a distinct systemic role of IRF5 in macrophages is the orchestration of transcriptional activation of pro-inflammatory cytokines, chemokines and co-stimulatory molecules leading to efficient effector T cell response, rather than induction of type I IFN-induced transcriptional network⁴⁹. Our data establish a new paradigm for macrophage polarization and designate the IRF5-IRF4 regulatory axis as a new target for therapeutic intervention: inhibition of IRF5 activity would specifically affect pro-inflammatory cytokine expression and decrease the number of effector T cells.

### REFERENCES FOR EXAMPLE 1

1. Gordon, S. & Taylor, P. R. Monocyte and macrophage heterogeneity. Nat Rev Immunol 5, 953-964 (2005).
2. Mosser, D. M. & Edwards, J. P. Exploring the full spectrum of macrophage activation. Nat Rev Immunol 8, 958-969 (2008).
3. Gordon, S. Alternative activation of macrophages. Nat Rev Immunol 3, 23-35 (2003).
4. Romagnani, P., Annunziato, F., Piccinni, M. P., Maggi, E. & Romagnani, S. Th1/Th2 cells, their associated molecules and role in pathophysiology. Eur Cytokine Netw 11, 510-511 (2000).
5. Martinez, F. O., Sica, A., Mantovani, A. & Locati, M. Macrophage activation and polarization. Front Biosci 13, 453-461 (2008).
6. Korn, T., Bettelli, E., Oukka, M. & Kuchroo, V. K. IL-17 and Th17 Cells. Annu Rev Immunol 27, 485-517 (2009).
7. Tamura, T. et al. IFN regulatory factor-4 and -8 govern dendritic cell subset development and their functional diversity. J Immunol 174, 2573-2581 (2005).
8. Porta, C. et al. Tolerance and M2 (alternative) macrophage polarization are related processes orchestrated by p50 nuclear factor kappaB. Proc Natl Acad Sci USA 106, 14978-14983 (2009).
9. Ruffell, D. et al. A CREB-C/EBPbeta cascade induces M2 macrophage-specific gene expression and promotes muscle injury repair. Proc Natl Acad Sci USA 106, 17475-17480 (2009).
10. Satoh, T. et al. The Jmjd3-Irf4 axis regulates M2 macrophage polarization and host responses against helminth infection. Nat Immunol 11, 936-944 (2010).
11. Ouyang, X. et al. Cooperation between MyD88 and TRIF pathways in TLR synergy via IRF5 activation. Biochem Biophys Res Commun 354, 1045-1051 (2007).
12. Takaoka, A. et al. Integral role of IRF-5 in the gene induction programme activated by Toll-like receptors. Nature 434, 243-249 (2005).
13. Mancl, M. E. et al. Two discrete promoters regulate the alternatively spliced human interferon regulatory factor-5 isoforms. Multiple isoforms with distinct cell type-specific expression, localization, regulation, and function. J Biol Chem 280, 21078-21090 (2005).
14. Dideberg, V. et al. An insertion-deletion polymorphism in the interferon regulatory Factor 5 (IRF5) gene confers risk of inflammatory bowel diseases. Hum Mol Genet. 16, 3008-3016 (2007).
15. Dieguez-Gonzalez, R. et al. Association of interferon regulatory factor 5 haplotypes, similar to that found in systemic lupus erythematosus, in a large subgroup of patients with rheumatoid arthritis. Arthritis Rheum 58, 1264-1274 (2008).
16. Graham, R. R. et al. A common haplotype of interferon regulatory factor 5 (IRF5) regulates splicing and expression and is associated with increased risk of systemic lupus erythematosus. Nat Genet. 38, 550-555 (2006).
17. Kristjansdottir, G. et al. Interferon regulatory factor 5 (IRF5) gene variants are associated with multiple sclerosis in three distinct populations. J Med Genet. 45, 362-369 (2008).
18. Miceli-Richard, C. et al. Association of an IRF5 gene functional polymorphism with Sjogren's syndrome. Arthritis Rheum 56, 3989-3994 (2007).
19. Fleetwood, A. J., Lawrence, T., Hamilton, J. A. & Cook, A. D. Granulocyte-macrophage colony-stimulating factor (CSF) and macrophage CSF-dependent macrophage phenotypes display differences in cytokine profiles and transcription factor activities: implications for CSF blockade in inflammation. J Immunol 178, 5245-5252 (2007).
20. Hoeve, M. A. et al. Divergent effects of IL-12 and IL-23 on the production of IL-17 by human T cells. Eur J Immunol 36, 661-670 (2006).
21. Verreck, F. A., de Boer, T., Langenberg, D. M., van der Zanden, L. & Ottenhoff, T. H. Phenotypic and functional profiling of human proinflammatory type-1 and anti-inflammatory type-2 macrophages in response to microbial antigens and IFN-gamma- and CD40L-mediated costimulation. J Leukoc Biol 79, 285-293 (2006).
22. Krausgruber, T. et al. IRF5 is required for late-phase TNF secretion by human dendritic cells. Blood 115, 4421-4430 (2010).
23. Hammer, M. et al. Dual specificity phosphatase 1 (DUSP1) regulates a subset of LPS-induced genes and protects mice from lethal endotoxin shock. J Exp Med 203, 15-20 (2006).
24. Fleetwood, A. J., Dinh, H., Cook, A. D., Hertzog, P. J. & Hamilton, J. A. GM-CSF- and M-CSF-dependent macrophage phenotypes display differential dependence on type I interferon signaling. J Leukoc Biol 86, 411-421 (2009).
25. Ahern, P. P. et al. Interleukin-23 drives intestinal inflammation through direct activity on T cells. Immunity 33, 279-288 (2010).
26. Nistala, K. et al. Th17 plasticity in human autoimmune arthritis is driven by the inflammatory environment. Proc Natl Acad Sci USA 107, 14751-14756 (2010).
27. Martinez, F. O., Gordon, S., Locati, M. & Mantovani, A. Transcriptional profiling of the human monocyte-to-macrophage differentiation and polarization: new molecules and patterns of gene expression. J Immunol 177, 7303-7311 (2006).
28. Ziegler-Heitbrock, L. et al. IFN-alpha induces the human IL-10 gene by recruiting both IFN regulatory factor 1 and Stat3. J Immunol 171, 285-290 (2003).
29. Hamilton, J. A. Colony-stimulating factors in inflammation and autoimmunity. Nat Rev Immunol 8, 533-544 (2008).
30. Medzhitov, R. & Horng, T. Transcriptional control of the inflammatory response.
Nat Rev Immunol 9, 692-703 (2009).
31. Ghisletti, S. et al. Identification and Characterization of Enhancers Controlling the Inflammatory Gene Expression Program in Macrophages. Immunity (2010).
32. Negishi, H. et al. Negative regulation of Toll-like-receptor signaling by IRF-4. Proc Natl Acad Sci USA 102, 15989-15994 (2005).
33. El Chartouni, C., Schwarzfischer, L. & Rehli, M. Interleukin-4 induced interferon regulatory factor (Irf) 4 participates in the regulation of alternative macrophage priming. Immunobiology 215, 821-825 (2010).
34. Sanjabi, S., Hoffmann, A., Liou, H. C., Baltimore, D. & Smale, S. T. Selective requirement for c-Rel during IL-12 P40 gene induction in macrophages. Proc Natl Acad Sci USA 97, 12705-12710 (2000).
35. Mise-Omata, S. et al. A proximal kappaB site in the IL-23 p19 promoter is responsible for RelA- and c-Rel-dependent transcription. J Immunol 179, 6596-6603 (2007).
36. Saraiva, M. & O'Garra, A. The regulation of IL-10 production by immune cells. Nat Rev Immunol 10, 170-181 (2010).
37. Fiorentino, D. F., Zlotnik, A., Mosmann, T. R., Howard, M. & O'Garra, A. IL-10 inhibits cytokine production by activated macrophages. J Immunol 147, 3815-3822 (1991).
38. Wing, K. & Sakaguchi, S. Regulatory T cells exert checks and balances on self tolerance and autoimmunity. Nat Immunol 11, 7-13 (2010).
39. Mosser, D. M. & Zhang, X. Interleukin-10: new perspectives on an old cytokine. Immunol Rev 226, 205-218 (2008).
40. Schneemann, M. & Schoeden, G. Macrophage biology and immunology: man is not a mouse. J Leukoc Biol 81, 579; discussion 580 (2007).
41. Ponting, C. P. The functional repertoires of metazoan genomes. Nat Rev Genet. 9, 689-698 (2008).
42. Oppmann, B. et al. Novel p19 protein engages IL-12p40 to form a cytokine, IL-23, with biological activities similar as well as distinct from IL-12. Immunity 13, 715-725 (2000).
43. Romagnani, S., Maggi, E., Liotta, F., Cosmi, L. & Annunziato, F. Properties and origin of human Th17 cells. Mol Immunol 47, 3-7 (2009).
44. Murphy, C. A. et al. Divergent pro- and antiinflammatory roles for IL-23 and IL-12 in joint autoimmune inflammation. J Exp Med 198, 1951-1957 (2003).
45. Yen, D. et al. IL-23 is essential for T cell-mediated colitis and promotes inflammation via IL-17 and IL-6. J Clin Invest 116, 1310-1316 (2006).
46. Shen, H. et al. Gender-dependent expression of murine Irf5 gene: implications for sex bias in autoimmunity. J Mol Cell Biol 2, 284-290 (2010).
47. Campbell, I. K. et al. Protection from collagen-induced arthritis in granulocyte-macrophage colony-stimulating factor-deficient mice. J Immunol 161, 3639-3644 (1998).
48. Cook, A. D., Braine, E. L., Campbell, I. K., Rich, M. J. & Hamilton, J. A. Blockade of collagen-induced arthritis post-onset by antibody to granulocyte-macrophage colony-stimulating factor (GM-CSF): requirement for GM-CSF in the effector phase of disease. Arthritis Res 3, 293-298 (2001).
49. Lacaze, P. et al. Combined genome-wide expression profiling and targeted RNA interference in primary mouse macrophages reveals perturbation of transcriptional networks associated with interferon signalling. BMC Genomics 10, 372 (2009).
50. Liu, J., Cao, S., Herman, L. M. & Ma, X. Differential regulation of interleukin (IL)-12 p35 and p40 gene expression and interferon (IFN)-gamma-primed IL-12 production by IFN regulatory factor 1. J Exp Med 198, 1265-1276 (2003).

### SUPPLEMENTARY REFERENCES FOR EXAMPLE 1

Team, R. D. C. in Foundation for Statistical Computing, Vol. Vienna Austria, 2010).
Huber, W., von Heydebreck, A., Sultmann, H., Poustka, A. & Vingron, M. Variance stabilization applied to microarray data calibration and to the quantification of differential expression. Bioinformatics 18 Suppl1, 596-104 (2002).
Smyth, G. K. et al. Limma: linear models for microarray data, in Bioinformatics and Computational Biology Solutions using Rand Bioconductor 397-420 (Springer, New York, 2005).
Benjamini, Y. & Hochberg, Y. Controlling the False Discovery Rate: A Practical and Powerful Approach to Multiple Testing. Journal of the Royal Statistical Society. Series B (Methodological) 57, 289-300 (1995).
Vlieghe, D. et al. A new generation of JASPAR, the open-access repository for transcription factor binding site profiles. Nucleic Acids Res 34, D95-97 (2006).
Asquith, D. L., Miller, A. M., McInnes, I. B. & Liew, F. Y. Animal models of rheumatoid arthritis. Eur J Immunol 39, 2040-2044 (2009).
Inglis, J. J., et al. Collagen-induced arthritis in C57BL/6 mice is associated with a robust and sustained T-cell response to type II collagen. Arthritis Res Ther 9, R113 (2007).

### EXAMPLE 2

### Irf5 is Required for Late-Phase TNF Secretion by Human Dendritic Cells

The information in Example 2 has been published by the inventors as Krausgruber et al “IRF5 is required for late-phase TNF secretion by human dendritic cells”. Blood 115: 4421-4430 (2010), incorporated herein by reference.

### Abstract

Spatially and temporally controlled expression of inflammatory mediators is critical for an appropriate immune response. In this study we define the role for Interferon Regulatory Factor 5 (IRF5) in secretion of Tumour Necrosis Factor (TNF) by human dendritic cells (DCs). We demonstrate that DCs but not macrophages have high levels of IRF5 protein and that IRF5 is responsible for the late phase expression of TNF, which is absent in macrophages. Sustained TNF secretion is essential for robust T cell activation by DCs. Systematic bioinformatic and biochemical analyses of the TNF gene locus map two sites of IRF5 recruitment: 5′ upstream and 3′ downstream of the TNF gene. Remarkably, while IRF5 can directly bind to DNA in the upstream region, its recruitment to the downstream region depends on the protein-protein interactions with NF-κB RelA. This study provides new insights into diverse molecular mechanisms employed by IRF5 to regulate gene expression and implicates RelA-IRF5 interactions as a putative target for cell-specific modulation of TNF expression.

### Introduction

TNF is one of the major cytokines responsible for effector immune functions. As well as playing a central role in host defence against infection, TNF is a major factor in the pathogenesis of chronic inflammatory disease such as rheumatoid arthritis (RA). Consequently, tightly controlled regulation of its expression is critical for an appropriate immune response. This occurs at the transcriptional and post-transcriptional levels, with transcriptional regulation showing specificity for both stimulus and cell type¹. The NF-κB family of transcription factors (TFs) plays a major role in transcriptional up-regulation of the TNF gene by lipopolysaccharide (LPS) in both mouse and human myeloid cells²⁻⁵.

Regulation of transcription for many immune genes in response to Toll-like receptor (TLR) signalling involves a combination of NF-κB and IRF factors⁶. IRFs appear to provide a mechanism for conferring signal specificity to a variety of target gene subsets, with IRF3 being essential for type I interferon (IFN) response⁷ and IRF5 playing a key role in induction of pro-inflammatory cytokines, including TNF, IL-6 and IL-12⁸. Consequently, IRF5^{-/-} mice show resistance to lethal shock induced by CpG-B or LPS⁸.

Unlike other IRF family members, IRF5 contains two nuclear localisation signals (NLS), one in the N- and the other in the C-terminus of the protein. This results in low levels of nuclear translocation and therefore weak trans-activation activity of IRF5 even in unstimulated cells⁹. The molecular pathways leading to IRF5 activation are not well understood, but it was shown that TLR signalling induces the formation of MyD88-IRF5-TRAF6 complexes⁸, and is probably followed by phosphorylation of specific sites within IRF5 C-terminal autoinhibitory domain¹⁰.

Human IRF5 is expressed as multiple spliced variants with distinct cell type-specific expression, cellular localization, differential regulation and dissimilar functions¹¹. Moreover, genetic polymorphisms in the IRF5 gene leading to expression of several unique isoforms have been implicated in autoimmune diseases including systemic lupus erythematosus (SLE), RA and Sjogren's syndrome¹²⁻¹⁵. IRF5 mRNA expression has been detected in B cells, DCs, monocytes, natural killer cells (NK) but not in T cells¹¹, yet little is known about the IRF5 protein expression in these cells.

Here we demonstrate that human monocytes acquire high levels of IRF5 protein during differentiation into dendritic cells (MDDCs) but not macrophages (MDMs). This leads to a sustained secretion of TNF by MDDCs compared to MDMs and efficient activation of T cells. IRF5 is recruited to both upstream and downstream regions of the gene following LPS induction and its cooperative action with NE-□B RelA is important for maintaining the TNF gene transcription. Remarkably, IRF5 displays two independent modes of transcriptional activity: direct binding to DNA and indirect recruitment via the formation of a protein complex with RelA. Our results provide novel insights into the molecular basis for cell specificity in TNF production by human immune cells and highlight RelA-IRF5 interactions as a novel target for cell-specific modulation of TNF expression.

### Material and Methods

### Plasmids

Expression constructs encoding full length human IRF3, IRF5v3/v4, and NF-κB subunits tagged with HA-tag in modified pENTR vector (pBent) were described in Ref¹⁶. IRF5ΔDBD, IRF5A68P mutants were generated. The constructs were recombined into pAD/PL DEST vector (Invitrogen, USA) for adenovirus production and subsequent delivery into human myeloid cells. IRF5-HA fragment was subsequently transferred into the modified pBent vector containing one-strep-tag. The 5′ wt/3′ wt and 5′ wt/3′ mut TNF luciferase-reporter constructs were used to generate 5′ mut/3′ wt and 5′ mut/3′ mut constructs with mutated sites κB2/2ξ/2a⁵. IRF5 DBD (aa 1-131) were PCR amplified and cloned into bacterial expression vector pET21d (Novagen, UK). All constructs were verified by sequencing.

### Cell Culture

All reagents used for cell culture were tested for endotoxin and only in use if the endotoxin levels were <20 pg/ml (Lonza, Switzerland). All cell cultures were maintained at 37° C. in 5% CO2 and 95% humidity in the appropriate media supplemented with 10% foetal calf serum (Gibco, USA) and 1% penicillin/streptomycin (PAA, USA). HEK-293-TLR4-CD14/Md2 cells (Invivogen, USA) were cultured in DMEM (PAA, USA) supplemented with 10 mg/ml of Blasticidin and 50 mg/ml of HygroGold™ (Invivogen, USA) as per manufacturer's instruction. Enriched populations of human monocytes were obtained from the blood of healthy donors by elutriation as described previously⁴. MDMs and MDDCs were obtained after 5-7 days of culturing human monocytes in RPMI 1640 (PAA, USA) supplemented with 100 ng/ml macrophage-colony stimulating factor (M-CSF) or 50 ng/ml granulocyte macrophage-colony stimulating factor (GM-CSF) and 10 ng/ml IL-4 (Peprotech, UK). MDMs, MDDCs and cell lines were stimulated with 100 ng/ml of LPS (Alexis Biochemicals, USA) unless indicated otherwise.

### ELISA

Cytokine secretion was quantified with specific ELISAs for human TNF (BD Bioscience), human IFN-γ (BD Bioscience) and human IFN-α1/IL-29 (R&D Systems) according to manufacturer's instructions. Absorbance was read at 450 nm by a spectrophotometric ELISA plate reader (Labsystems Multiscan Biochromic) and analysed using Ascent Labsystems software. All samples were analysed in triplicate in a volume of 50p1.

### Mixed Lymphocyte Reaction

Human MDDCs were plated in 96-well, flat-bottom tissue plates at 2×10⁴ cells/well. T lymphocytes were isolated form the blood of healthy donors by elutriation, analysed by FACS and used if purity was >90%. T lymphocytes were added to MDDCs at 5×10⁵, such as the final MDDC:T cell ratio was 1:25. Control cultures contained medium or T lymphocytes, or MDDCs alone. 10 μg/ml of anti-TNFR1 antibody (MAB 625, R&D Systems) or IgG control antibody (MAB 002, R&D Systems) was added to the co-cultures after 6 h or 24 h. Cultures were established in duplicate and incubated at 37° C. in 5%. CO₂ for a total of 72 h. Following culture, supernatants were collected and stored at −20° C. for detection of cytokines.

### RNA Interference

siRNA-mediated knockdown was performed using On-target plus SMART pool reagents (Dharmacon, USA) designed to target human IRF5 and NF-κB RelA. Lipofectamine™ RNAiMAX (Invitrogen, USA) and DharmaFECT I® (Dharmacon, USA) were employed as the siRNAs transfection reagents for HEK-293-TLR4-Md2/CD14 cells and MDDCs respectively according to manufacturers' instructions. Multiple siRNAs were used to validate the knock-down specificity and exclude off target effects.

### Adenoviral Infection

Infections of MDMs were performed in 96-well plates in triplicate at a multiplicity of infection of 50:1. Cells were seeded in serum-free, antibiotics-free RPMI containing the desired number of viral particles in a final volume of 50:1. The plates were incubated overnight at 37° C. followed by aspiration of the supernatants and replacement with 100 μl of standard media per well. Cells were allowed to recover for a further 24 hours before experimental assay.

RNA Extraction and Quantitative Real-Time RT-PCR

Total RNA was extracted from cells using a QiaAmp RNA Blood mini kit (Qiagen, Germany) according to manufacturer's instructions. cDNA was synthesised from total RNA using SuperScript® III Reverse Transcriptase (Invitrogen, USA) and 18-mer oligo dTs (Eurofins MWG Operon, UK). The gene expression was analysed by 2-standard curve or ΔΔCt methods where appropriate based on the quantitative real-time PCR with TaqMan primer sets for human TNF and PO (Applied Biosystems) in a Corbett Rotor-gene 6000 machine (Corbett Research Ltd, UK).

### Luciferase Gene Reporter Assay

HEK-293-TLR4-CD14/Md2 cells were seeded into poly-lysine coated 96-well plates at a density of 30,000 cells per well. Next day, cells were transfected with 10 ng of the indicated expression vector, 50 ng of TNF luciferase reporter and 50 ng of pEAK8-Renilla using Lipofectamine 2000 protocol (Invitrogen). Total amount of DNA was kept 120 ng per well. 48 hours after transfection the activity of the reporters were measured using Dual-Glo Luciferase system (Promega, USA) optimized for 96-well plate format according to the manufacturer's protocol. Each experiment was performed in triplicate.

### Nuclear and Total Protein Extracts and Western

Cells were grown on 10 cm² dishes, exposed to vehicle, agents and reactions terminated by washing cells twice with ice cold PBS. Cells were then removed by scraping and transferred to Eppendorf tubes. Nuclear or total protein extracts were prepared as previously described⁵. Equal amounts of proteins were resolved by Novex Tris-glycine gel (Invitrogen, USA), transferred onto Hybond-N membranes (Amersham Biosciences, USA) and subjected to incubation with antibodies against IRF5 (ab2932, Abcam, UK) followed by followed by detection with HRP-conjugated secondary antibodies and the chemiluminescent substrate solution ECL (GE Healthcare, USA).

EMSA
Oligonucleotide probes were radiolabeled with
[α-32P]dCTP (Perkin Elmer, USA):

| | | |
|---|---|---|
| κB4 | F: | agctGGGCATGGGAATTTCCAACTCT; |
| | | (SEQ ID No: 30) |
| | R: | agctGAGTTGGAAATTCCCATGCCC; |
| | | (SEQ ID No: 31) |
| κB4a | F: | agctAACTCTGGGAATTCCAATCCTT; |
| | | (SEQ ID No: 32) |
| | R: | agctAAGGATTGGAATTCCCAGAGT T; |
| | | (SEQ ID No: 33) |
| κB4b | F: | agctCTTGCTGGGAAAATCCTGCAG; |
| | | (SEQ ID No: 34) |
| | R: | agctGCTGCAGGATTTTCCCAGCA AG; |
| | | (SEQ ID No: 35) |
| ISRE1 | F: | agctGAAGCCAAGACTGAAACCAGCATTA; |
| | | (SEQ ID No: 36) |
| | R: | agctTAATGCTGGTTT CAGTCTTGGCTTC; |
| | | (SEQ ID No: 37) |
| ISRE2 | F: | agctCCGGGTCAGAATGAAAGAAGAAGG; |
| | | (SEQ ID No: 38) |
| | R: | agctC CTTCTTCTTTCATTCTGACC CGGT; |
| | | (SEQ ID No: 39) |
| ISRE5 | F: | agctGGAGAAGAAACCGAGACAGAAGG TG; |
| | | (SEQ ID No: 40) |
| | R: | agctCACCTTCTGTC TCGGTTTCTTCTCC; |
| | | (SEQ ID No: 41) |
| ‘ISRE’16 | F: | agctTTTGCTTAGAAAAGAAACATGGTCTC; |
| | | (SEQ ID No: 42) |
| | R: | agctGAG ACCATGTTTCTTTTCTAAGCAAA; |
| | | (SEQ ID No: 43) |
| ‘ISRE’17 | F: | agctACATAAACAAAGCCCAACAGAATAT TCC; |
| | | (SEQ ID No: 44) |
| | R: | agctGGAATATTCTGTTGGGCTTTGTTTATGT; |
| | | (SEQ ID No: 45) |
| | | and |
| PRDI-III | F: | agctGGGAAACTG AAAGGGAAAGTGAAAGTGG; |
| (IFN- [symbol] | | (SEQ ID No: 46) |
| | R: | agctCCACTTTCACTTTCCCTTTCAGTTTCCC. |
| | | (SEQ ID No: 47) |

Binding reactions with 50 ng of bacterially expressed and purified IRF5 DBD and EMSA gel separation were performed as previously described⁵.

### Chromatin Immunoprecipitation

7×10⁶ MDDCs or 293-TLR4 cells were fixed by adding 1% formaldehyde (final concentration) for 5 minutes at room temperature. Nuclear extracts were subjected to 6×12 second pulses of sonication using Vibra-Cell VCX130 (Sonics, USA) at 20% amplitude. For immuno-precipitation reaction nuclear extracts were pre-cleared with Protein G Sepharose™ bead slurry (GE Healthcare) for 2 h, and then incubated with 2 μg of antibodies against IRF5 (ab2932, Abcam, UK), RelA or Pol II (sc-372 and sc-899, Santa Cruz, USA) overnight at 4° C. with rotation. Immuno-complexes were then collected with protein A sepharose beads for 30 minutes, rigorously washed and eluted. For Re-ChIP RelA ChIP eluates were subsequently incubated with either IRF5 antibody or no antibody control and processed as above. Cross-linked protein-DNA complexes were reversed by incubating them at 65° C. overnight and DNA fragments were purified using the QIAquick PCR Purification Kit (Qiagen, Germany). The immuno-precipitated DNA fragments were then interrogated by real-time PCR using SYBR®Premix Ex Taq II™ master mix (Takara Bio, USA) and the following primers:

TNF locus: control region
(SEQ ID No: 48)
(TGTGTGTCTGGGAGTGAGAACT
and
(SEQ ID No: 49)
TCTTCTCAGCTTCTCCTTTGCT),
region A
(SEQ ID No: 50)
(CCACAGCAATGGGTAGGAGAATGT
and
(SEQ ID No: 51)
GAGGTCCTGGAGGCTCTTTCACT),
region B
(SEQ ID No: 52)
(GGAAGCCAAGACTGAAACCAGCA
and
(SEQ ID No: 53)
CCGGGAATTCACAGACCCCACT),
region C
(SEQ ID No: 54)
(TCCCTCCAACCCCGTTTTCT
and
(SEQ ID No: 55)
TAGGACCCTGGAGGCTGAAC),
region D
(SEQ ID No: 56)
(AACTTTCCAAATCCCCGCCC
and
(SEQ ID No: 57)
GGTGTGCCAACAACTGCCTT),
region E
(SEQ ID No: 58)
(CAGCAAGGACAGCAGAGGAC
and
(SEQ ID No: 59)
TCCCGGATCATGCTTTCAGT),
region F
(SEQ ID No: 60)
(GGCAGTCAGTAAGTGTCTCCAA
and
(SEQ ID No: 61)
TACCTACAACATGGGCTACAGG),
region G
(SEQ ID No: 62)
(ACAGCTTTGATCCCTGACATCT
and
(SEQ ID No: 63)
CTCCGTGTCTCAAGGAAGTCTG),
region H
(SEQ ID No: 64)
(ATATTCCCCATCCCCCAGGAAACA
and
(SEQ ID No: 65)
CTGCAACAGCCGGAAATCTCACC),
region I
(SEQ ID No: 66)
(GAGGACCTCACTCAGCCCTT
and
(SEQ ID No: 67)
CGGCAGTTCGGTTCCTTGTT),
region J
(SEQ ID No: 68)
(ACTGGTCTTTGTGGTGAAGGAG
and
(SEQ ID No: 69)
GAACTAGTGGGCTCAAGTGGTC),
region K
(SEQ ID No: 70)
(GCTATGATCATGCCACTGTACCC
and
(SEQ ID No: 71)
TACCACATGGTTTTCTCCTGCC),
region L
(SEQ ID No: 72)
(GCTGAAAGTCAGCCATGAAGTA
and
(SEQ ID No: 73)
CACTTAGGGTGTCCCATTTAGG).

Data were analysed using Rotogene 6000 software (Corbett Research Ltd, UK). All primer sets were tested for specificity and equal efficiency before use.

### Immunoprecipitation

HEK-293-TLR4-CD14/Md2 cells were transfected with onestrep-IRF5-HA construct or corresponding empty vector. 24 hrs post transfection cells were fixed with 1% formaldehyde for 10 minutes at room temperature prior to high salt lysis and affinity purification on Strep-Tactin MacroPrep sepharose (IBA). The eluates were de-crosslinked by incubating at 65° C. overnight prior to separation by SDS-PAGE. Exogenous IRF5 and endogenous RelA were detected by immunoblotting with anti-HA-HRP (12013819001, Roche) and anti-RelA (sc-372, Santa-Cruz, US). Alternatively, cells were transfected with RelA-FLAG or BAP-FLAG control protein. 24 hrs post transfection cells were lysed and affinity purified with anti-FLAG M2 sepharose beads (Sigma, UK). Exogenous RelA and endogenous IRF5 were detected by immunoblotting with anti-FLAG-HRP (A8592, Sigma, UK) and anti-IRF5 (Abcam, UK). Interaction of endogenous RelA and IRF5 was detected by overnight incubation of the cell lysates with goat anti-IRF5 antibody (ab2932, Abcam, UK) or no antibody control prior to precipitation with protein G beads. IRF5 was detected by immunoblotting with mouse anti-IRF5 antibody (sc-56714, Santa-Cruz, USA) while RelA—by immunoblotting with anti-RelA. Triton X-100 extracted nuclei and DNase I digestion of chromatin was performed as described previously¹⁷.

### Bioinformatics and Statistical Analyses

The nucleotide sequence were inspected with transcription factor binding site searching software JASPAR (http://jaspar.cgb.ki.se/)¹⁸ and Genomatrix (http://www.genomatix.de/) for the presence of putative ISRE sites (FIG. 6). Statistical analysis was performed using One-way ANOVA with Dunnett's multiple comparison post test or Student's T-test where appropriate (*p<0.05, **p<0.01, ***p<0.001).

### Results

### Sustained TNF Secretion by MDDCs is Important for Efficient T Cell Activation

Myeloid cells (e.g. macrophages and DCs) are the major producers of the key immune modulator TNF in response to TLR4 stimulation¹⁹. TNF protein is below the limit of detection in the supernatants of resting cells (FIG. 17A). Following 4 h of LPS stimulation, TNF is secreted at similar levels in MDMs and MDDCs (early phase). However, a marked difference in TNF production was observed in MDMs and MDDCs stimulated with LPS for 24 h (late phase). While the level of TNF significantly decreased in MDMs, there was an increase in TNF levels in MDDCs (FIG. 17A) in each individual blood donor (FIG. 18A).

Human TNF acting through TNF receptor is involved in DC maturation from bone marrow precursors^{20,21} and activation of type I helper (Th1), measured by the release of Interferon gamma (IFN-γ)²². Thus, we examined weather the late phase secretion of TNF by MDDCs is needed for IFN-γ production by T cells. MDDCs were stimulated with LPS for 2 h and exposed to human T cells extracted and purified from peripheral blood of major histocompatibility complex (MHC) unmatched donors in a mixed lymphocyte reaction (MLR). Antibodies against TNF receptor 1 (TNFR1) or isotype IgG control were added to the reaction. T cells incubated with MDDCs treated with anti-IgG antibodies produced high levels of IFN-γ (FIG. 17B), while the control reactions (MDDCs or T cells cultured on their own) secreted no detectable IFN-γ. Blocking TNF at 6 h after setting the MLR reaction resulted in strong reduction of IFN-γ, but no effect was observed when anti-TNFR1 antibodies were added to the reaction after 24 h, suggesting that most of T cells are in activated state after the prolonged exposure to TNF (FIG. 17B).

Thus, the observed sustained expression of TNF by MDDCs which might be of benefit to both their maturation and antigen presenting function and is essential for establishing a robust Th1 phenotype.

IRF5 Protein is Highly Expressed in MDDC and Controls Late Phase TNF Secretion

The observed differential LPS-induced secretion of TNF by human DCs and macrophages (FIG. 17A) prompted us to examine the molecular mechanisms of this phenomenon. Myeloid cells from IRF5^{-/-} mice show impaired induction of pro-inflammatory cytokines including TNF upon stimulation by different TLR ligands⁸. We hypothesised that the difference in TNF secretion profile in MDDCs and MDMs might be due to the difference in IRF5 expression in these cells. We examined levels of IRF5 protein following human monocyte differentiation into MDMs and MDDCs. No increase in the levels of IRF5 protein was observed in MDMs even after 5 days of differentiation (FIG. 17C). However, expression of IRF5 protein was detected following 1 day of monocyte differentiation into MDDCs and remained at an elevated level until day 7 (FIG. 17C). Significantly, whereas at least three different IRF5 isoforms were observed in human monocytes, only some of them accounted for high levels of IRF5 in MDDCs: one is likely to be IRF5v3/v4¹¹.

Next, we looked at the effect of ectopic IRF5 expression in MDMs that have low level of endogenous IRF5 protein (FIG. 17C) on T cell activation. MDMs were infected with adenoviral expression vector encoding HA-tagged IRF5 or the corresponding empty vector pBent. 48 h post infection no significant effect on the resting cells (measured by endogenous IFN-□1 response) was observed. Exposure of T cells to MDMs with elevated levels of IRF5 levels resulted in increase of IFN-λ secretion to the levels comparable to T cells exposed to MDDCs (FIG. 17D). Thus, we argued that IRF5 may be responsible for sustained secretion of TNF. To test this hypothesis, MDMs were infected with adenoviral expression vector encoding HA-tagged IRF5 or IRF3 (as a control) or pBent. IRF5-HA and IRF3-HA vectors expressed similar levels of proteins (FIG. 18B), but only IRF5 resulted in a significant increase in TNF secretion (FIGS. 17D, 18C), while only IRF3 induced IFN-α1 (FIG. 18D), consistent with the previously published data¹⁶. Strikingly, TNF secretion in MDMs with over-expression of IRF5 remained at a steady sustained level up to 48 h post LPS stimulation (FIG. 17E), similar to that of MDDCs with high levels of endogenous IRF5 (FIG. 17C). siRNA-mediated inhibition of IRF5 in MDDCs (FIG. 18E) resulted in reduction of TNF secretion at 8 and 24 h post LPS stimulation (FIG. 17F), supporting the notion that IRF5 may be required for the late phase TNF expression.

Taken together, these results suggest that sustained TNF secretion by MDDCs leading to robust T cell activation is likely to be a consequence of a high level of IRF5 protein in these cells.

### IRF5 is Involved in Transcriptional Regulation of TNF

We next sought to investigate whether IRF5 is involved in transcriptional regulation of the TNF gene. In human MDDCs stimulation with LPS resulted in a rapid up-regulation of TNF mRNA expression, which reached the peak between 1 and 2 h but remained at a steady level until 8 h post stimulation (FIG. 19A). Consistent with the observed differences in protein secretion, TNF mRNA expression in MDMs was characterised by more transient kinetics (FIG. 20A), while siRNA-mediated inhibition of IRF5 reduced TNF mRNA expression (FIG. 19A). The observed inhibition was statistically significant when analysed in multiple blood donors (FIG. 20B). In the same cells siRNA-mediated inhibition of NF-κB RelA, a transcription factor previously shown to be important for an efficient TNF production by human MDDCs²³, resulted in reduction of TNF mRNA expression at the initial phase of gene induction (1-2 h post LPS stimulation) (FIG. 19A). Within this time window, depletion of both IRF5 and RelA had the strongest effect on mRNA expression (FIG. 19A), indicating that RelA and IRF5 may cooperate in controlling transcription of the TNF gene.

To investigate whether IRF5 can directly modulate transcription of the TNF gene, we used a gene-reporter plasmid in which the luciferase gene was flanked with 1171 nt 5′ upstream and 1252 nt 3′ downstream of the TNF gene. This construct encompassed all evolutionary conserved sequences in the region and contained known κB sites^{24,25}. It was co-expressed with HA-tagged IRF5, IRF3 and NF-κB subunits in HEK-293 cells, and luciferase activities were compared to empty vector pBent. RelA and IRF5 transfected cells showed a significant increase in luciferase activity (FIG. 19B). Other NF-κB subunits or IRF3 had little or no effect (FIGS. 19B, 20C). Of interest, a deletion of the IRF5 DNA-binding domain (IRF5 ΔDBD) or a point alanine to proline mutation in it (IRF5 A68P) previously shown to act as dominant negative mutants of IRF5^{26,27}, resulted in a major drop in luciferase activity (FIG. 20D).

We concluded that IRF5 along with RelA is likely to be directly involved in the transcriptional regulation of the human TNF gene. While the initial phase of TNF induction depends on both factors, only IRF5 appears to be crucial for maintaining prolonged TNF transcription in MDDCs. Moreover, the DBD of IRF5 is required for the optimal level of TNF gene up-regulation.

### IRF5 is Recruited to the 5′ Upstream and 3′ Downstream Regions of the TNF Gene in Response to LPS Stimulation

To further address the involvement of IRF5 in the TNF gene regulation, we systematically analysed the recruitment of IRF5 to the TNF locus. A well-conserved N-terminal DBD of IRF factors recognises a class of DNA sequences termed IFN-stimulated response element (ISRE), 15 of which were computationally mapped to this locus together with known κB sites: κB1, κB2/ζ/2a, κB3 and κB4/4a/4b (FIG. 21A and FIG. 22). A series of primers spanning the locus and encompassing ISRE sites was designed and used in the quantitative ChIP assay (FIG. 21A).

HEK-293-TLR4-CD14/Md2 cells responsive to LPS were used to investigate the effect of LPS stimulation on recruitment of IRF5 to the TNF locus. Increased occupancy of IRF5 was observed at regions A, B, C, G and H 4 h post LPS stimulation followed by a decrease after 24 h (FIG. 21B). Taking into consideration the average ChIP fragment size of around 500 by and the close proximity of the sequences amplified, some degree of overlap in regions A-C was inevitable and might have accounted for the observed symmetrical distribution of enrichment at regions A, B and C. While the enrichment of IRF5 signal at region B was expected due to the presence of putative ISRE 1/2 that can interact with IRF5 in vitro (FIG. 23A), it was surprising to observe the recruitment of IRF5 at region H since this region contains no putative ISREs (FIG. 21A). Moreover, we observed no IRF5-DNA binding at κB4/4a/4b binding sites in region H or at additional non-consensus ‘ISRE’16 and ‘ISRE’17 sites in the vicinity of region H (FIG. 22 and FIG. 23A). We also investigated the recruitment of NE-κB RelA to the TNF locus and observed LPS-induced binding of RelA to regions B, E and H (FIG. 21C), which correlated with the distribution of multiple NF-κB binding regions.

Next, we validated the pattern of IRF5 and RelA binding to the TNF locus in MDDCs stimulated with LPS for 0, 1 and 4 hours. Strong enrichment in both IRF5 and RelA recruitment was observed at regions B and H (FIG. 25D and FIG. 25E), reproducible in five independent blood donors (FIG. 23B). Importantly, LPS stimulation resulted in rapid transcription of the full length nascent TNF transcript (estimated by recruitment of Pol II to the TNF 3′ downstream region H), which was robustly maintained at least up to 4 hours post stimulation (FIG. 23D).

In summary, in response to LPS stimulation IRF5 along with RelA is efficiently recruited to the 5′ upstream and 3′ downstream regions of the human TNF gene. Significantly, the lack of putative ISRE binding sites in the 3′ downstream region of the gene strongly suggested that recruitment of IRF5 to this region may be mediated via its interactions with other TFs or accessory proteins.

IRF5 Forms Specific Physical Interactions with RelA

To tease out whether IRF5 recruitment to region H may be mediated via its interactions with RelA, we performed sequential ChIP analysis of the region and found that IRF5 recruitment was co-dependent on RelA following LPS stimulation (FIG. 21F). This finding prompted us to investigate whether IRF5 and RelA interact physically.

IRF5 with an N-terminal one-strep tag and a C-terminal HA tag was expressed in HEK-293 cells. FIG. 24 shows that in conditions similar to ChIP analysis (i.e. in-vivo crosslinking with formaldehyde) ectopically expressed IRF5, purified over a streptactin column, efficiently pulls down endogenous RelA (FIG. 24A, compare lanes 3 and 4). To determine whether this interaction was specific, we immunoblotted for other NE-κB family members: Rel-B, c-Rel, p50 and p52, or a control protein tubulin. None of these resulted in a positive interaction (FIG. 24A). Furthermore we conducted a complementary experiment, in which human RelA containing C-terminal FLAG tag was expressed in HEK-293 cells and immunoprecipitated in the absence of a cross-linking agents on anti-FLAG sepharose. Specific interactions between ectopically expressed RelA and endogenous IRF5 were observed. No interaction was detected between a control FLAG-tagged bacterial alkaline phosphatase (BAP) and IRF5 (FIG. 24B, compare lanes 1 and 2).

Next, we examined whether an interaction between the endogenous RelA and IRF5 could be detected in MDDCs and if this interaction may be induced by LPS stimulation. IRF5 was immunoprecipitated from the cells stimulated with LPS for 0 or 1 h using anti-IRF5 antibodies. The Western blot for RelA revealed a specific interaction with IRF5 (FIG. 24C). A densitometry analysis of quantities of the bait and target proteins indicated that the quantity of RelA bound to IRF5 was somewhat higher in LPS stimulated cells (FIG. 24C, lane 4).

Finally, we asked the question whether the observed RelA-IRF5 interactions are dependent on the simultaneous binding of both TFs to DNA, i.e. RelA and IRF5 interact only when bound to corresponding □B and ISRE binding sites in close proximity to each other. To address this, we extracted nuclei from HEK-293-TLR4-CD14/Md2 cells stimulated with LPS for 0, 1, 4 and 8 h and subjected the chromatin to DNase I digestion. Subsequent precipitation of endogenous immune complexes with anti-IRF5 antibodies revealed that RelA interacted with IRF5 even in the absence of DNA bridging (FIG. 24D, lanes 5-8). Once again, the number of RelA-IRF5 complexes increased with LPS stimulation, corresponding to the rise in nuclear RelA (FIG. 24D, lanes 1-4).

In summary, IRF5 can specifically interact with RelA but not other four NF-κB subunits. This interaction is not dependent on IRF5 binding to DNA and the quantity of RelA-IRF5 complexes is increased in response to LPS stimulation. Thus, we hypothesised that IRF5 recruitment to the 3′ downstream region of the TNF gene lacking putative ISRE sites is a consequence of direct physical interactions between DNA-bound RelA and IRF5.

### RelA is Required for IRF5-Dependent Trans-Activation of the TNF Gene

To test the above hypothesis, we first analysed IRF5 recruitment to the TNF locus in the cells in which the levels of RelA were significantly reduced. In HEK-293-TLR4-CD14/Md2 cells siRNA-mediated knockdown of RelA resulted in approximately 75% reduction in RelA protein (FIG. 25A) and about a 10-fold decline in its recruitment to region H following 4 h of LPS stimulation (FIG. 25B). As predicted, the IRF5 recruitment to the same region was prevented (FIG. 25C). Of interest, when we analysed RelA and IRF5 recruitment to region B in RelA-depleted cells, we observed only partial reduction in IRF5 recruitment (FIG. 26), consistent with the view that IRF5 can bind directly to DNA at this region (FIG. 23A).

Next, we examined the effect of site-specific mutations in the κB sites on the ability of IRF5 to activate the TNF gene. A panel of four gene-reporter constructs was used in this analysis (1) 5′ wt/3′ wt (as in FIG. 18B); 5′ mut/3′ wt (mutated κB2/2/2a sites in the TNF 5′ upstream); (3) 5′ wt/3′ mut (mutated κB4/4a sites in the 3′ TNF downstream); (4) 5′ mut/3′ mut (mutated all κB sites described above). The reporter constructs were co-expressed with HA-tagged IRF5 and RelA in HEK-293 cells, and luciferase activities were compared to empty vector pBent. As expected, removal of either 5′ upstream or 3′ downstream κB sites diminished the ability of RelA to drive the gene-reporter activity (FIG. 25D). However, the trans-activation of the reporter constructs by IRF5 (supported by ectopically expressed Myd88) appeared to be largely unaffected by mutations in the 5′ upstream □B sites, suggesting that IRF5 does not utilise κB2/2ξ/2a sites for its binding to the TNF 5′ upstream and its likely to involve the identified ISRE 1 and 2 sites. However, the trans-activation of the reporter construct with mutations in κB4/4a sites by IRF5 was significantly reduced, indicating that IRF5 activity depends on NF-κB binding to this region (FIG. 25B). Low amounts of endogenous RelA detected in the nuclei of resting HEK-293-TLR4-CD14/Md2 cells (data not shown) appeared to provide a necessary DNA-anchor for IRF5.

Thus, IRF5 recruitment to the TNF 3′ downstream region is mediated by way of a complex assembly with RelA and does not involve a direct contact to DNA. Importantly, another mode of function of IRF5 in TNF regulation is a direct recruitment to the TNF gene 5′ upstream. The two functional modes also imply the possibility of a higher order enhancer structure at the TNF locus, possibly involving IRF5-RelA mediated intrachromosomal looping.

### Discussion

Production of the key immune modulator TNF is both cell- and stimulus-specific. Myeloid cells are the major producers of TNF in response to TLR4 stimulation¹⁹. Consequently, a tight control of the amount and duration of TNF expression by these cells is critical for a self-limited immune response. Here we aimed to understand the molecular bases of differential TNF expression in human dendritic cells and macrophages. We demonstrate that IRF5 appears to be a defining factor in maintaining the TNF gene transcription in MDDCs. Remarkably, we unravel a complex molecular mechanism employed by IRF5 to control the human TNF gene expression: two spatially separated regulatory regions and two independent modes of actions are involved.

IRF5 is highly expressed in MDDCs but not other myeloid cells (FIG. 17). During differentiation MDDCs acquire a particular phenotype, characterized among other markers by higher levels of RelB and c-Rel²⁸. Important for understanding the mechanisms of sustained TNF expression, RelB was previously shown to replace RelA at the promoters of macrophage derived chemokine and EBV-induced molecule 1 ligand chemokine genes and to prolong their transcription in MDDCs²⁹. We also observed an increase in the RelB and c-Rel levels during monocyte differentiation into MDDCs, but not into MDMs (FIG. 27A). However, neither RelB nor c-Rel was able to drive transcription of TNF (FIG. 20), whereas RelA, whose level was similar in all human myeloid cell types (FIG. 27A), had a strong trans-activating effect (FIG. 19). This led us to conclude that RelA was likely to participate in initial phase of TNF activation, which is indistinguishable between MDDCs and MDMs, but other MDDC-specific factors, such as IRF5, may contribute to the observed extended expression of TNF in MDDCs. Indeed, forced expression of IRF5 in MDMs led to prolonged TNF secretion (FIG. 17), while depletion of IRF5 in MDDCs resulted in reduction of TNF expression, particularly at later time (4 h) post LPS stimulation (FIGS. 19 and 4). Although we can not formally rule out other factors that might feed into the TNF expression system at a later time, the ability of IRF5 to activate the TNF gene-reporter construct (FIG. 19) and its efficient recruitment to the TNF locus (FIG. 21) strongly suggest a direct role for IRF5 in TNF gene induction in response to LPS.

TNF is an early primary response gene, whose mRNA expression in MDDCs is induced ˜100-fold within 30 min post LPS treatment (FIG. 24). The genomic locus encompassing the TNF gene is open to regulatory proteins and in murine bone marrow-derived macrophages (BMDMs) does not require nucleosome remodelling complexes for its activation³⁰. Consistent with this notion, we find a significant accumulation of Pol II molecules at the transcription start site (TSS) of the gene even in resting MDDCs (FIG. 28), akin to the results obtained in mouse BMDMs^{31,32}. LPS stimulation, however, results in a robust recruitment of RelA and IRF5 to both 5′ upstream region B and 3′ downstream region H (FIG. 21) and in a significant induction of Pol II recruitment to the 3′ downstream region of the gene (FIG. 28). This suggests an increase in production of full length nascent TNF transcripts upon LPS stimulation of MDDCs, in addition to induction of splicing of already generated nascent transcripts reported by Hargreaves et al³¹.

The recruitment of IRF5 to the 5′ upstream region is likely to involve direct binding to DNA via the identified ISRE sites (FIG. 23), whereas the recruitment of IRF5 to the 3′ downstream region is mediated via protein-protein interactions with RelA (FIG. 24). These interactions are induced following stimulation of MDDCs with LPS, while no other NE-κB subunits appear to complex with IRF5 (FIG. 24). Previous studies demonstrated that IRF3, another member of the IRF family, forms in vitro interactions with RelA via its Rel homology domain (RHD)³³. Considering that the RHD domain is a highly conserved domain present in all NF-κB proteins, the exclusiveness of IRF5 interactions with RelA is somewhat surprising. Further work is needed to map the interface of RelA-IRF5 interactions.

Regions B and H are characterised by high level of sequence conservation^{24,34}, and contain cell type-specific DNasel hypersensitivity sites^{34,35}. Moreover, the TNF 5′ upstream and 3′ downstream regions have been shown to physically interact by forming an intrachromosomal loop, the topology that could promote the re-initiation of transcription³⁴. This model may be of a particular relevance to TNF expression by MDDCs, in which a cooperative action of RelA and IRF5 at both the 5′ upstream and downstream regions appears to be essential for maintaining TNF gene transcription over a prolonged period of time. Here the locus circularization may be directed via newly unravelled protein-protein interactions between RelA and IRF5 (FIG. 24). The observed DNA binding-independent co-recruitment of IRF5 to the 3′ downstream region (FIG. 25) further supports the possibility of high-order enhancer structure at the locus (FIG. 29).

Why is TNF secretion maintained for longer in MDDCs than in MDMs (FIG. 17)? Dendritic cells are professional antigen-presenting cells (APC) that are crucial for both innate and adaptive responses to infection. They sense invading pathogens and respond by secreting various cytokines as well as by upregulating the expression of major histocompatibility complex II (MHC II) and costimulatory molecules, essential for efficient antigen presentation to T cells³⁶. The mature dendritic cells migrate to the draining lymph nodes, where they initiate Th1 differentiation. TNF acting through TNF receptor is involved in DC maturation from bone marrow precursors^{20,21}. Recent study demonstrated that TNF blockade impaired DC survival and function in RA³⁷. Our data showing that TNF produced by DCs is a key factor in human Th1 activation support this study. Moreover, it is the late phase TNF secretion that is needed to achieve the full activation potential (FIG. 17). Macrophages, on the other hand, do not migrate to the draining lymph nodes but accumulate in large numbers at a site of inflammation, secrete inflammatory cytokines and attract other immune cells via chemotaxis³⁸. Thus, a mechanism which would restrain the degree and duration of TNF secretion by macrophages would be important for ensuring resolution of acute inflammatory response thereby limiting tissue damage.

Another question is how IRF5 is activated in MDDCs by TLR4 signalling. Takaoka et al demonstrated that ectopically expressed IRF5 translocates to the cell nuclei in response to LPS, and this translocation is dependent on the presence of Myd88⁸. We observed endogenous nuclear IRF5 even in resting MDDCs or HEK-293-TLR4-CD14/Md2 cells, and its level was not increased following LPS stimulation (FIG. 27B), although we could not exclude the possibility of active nuclear export-import of IRF5 induced by phosphorylation at the previously described serine residues³⁹ due to the lack of phospho-specific antibodies. In the same cells endogenous IRF3 showed a clear pattern of induced nuclear translocation (FIG. 27B), which corresponded to its phosphorylated form (data not shown). It is worth noting however, that an ectopically expressed mutant of IRF5 in which the described critical serines at positions 427 and 430^{9,39} were substituted with alanines, was still transcriptionally active in the TNF reporter assay, suggesting that IRF5 may not need to be phosphorylated at these residues to activate transcription. We also did not observe any loss in IRF5 trans-activating potential when lysines 401 and 402, implicated in another Myd88-induced post-translational modification of IRF5, K63-linked polyubiquitination⁴⁰, were substituted with arginines. Since the over-expression data generated in HEK-293 cell line may be misleading, we plan to test these and other mutants of IRF5 in complementation experiments in the cells from IRF5-deficient mice to elucidate the impact of these mutations on IRF5 activation and function in vivo.

Regulation of IRF5 activity is an important issue since the excessive activation of this protein may lead to pathology. Interestingly, another member of the IRF family, IRF4, was shown to act as a negative regulator of TLR signalling by inhibiting the production of selected IRF5-dependent genes, including TNF, via direct competition with IRF5 for interactions with Myd88⁴¹. In mice, IRF4 was observed to be differentially expressed in DCs and regulate the development of a specific DC subset, conventional DCs⁴². In humans, IRF4 was also found to be expressed in MDDCs but not MDMs⁴³, suggesting that a self-controlled IRF5-IRF4 regulatory system might have developed to finely modulate TLR signalling pathways and production of IRF5-dependent inflammatory cytokines.

In summary, sustained TNF secretion in human MDDCs is mediated by cooperative action of IRF5 and RelA at the 5′ upstream and 3′ downstream regions of the TNF gene. TLR4 stimulation induces protein-protein interactions between RelA and IRF5 and allows for DNA-independent recruitment of IRF5 to the TNF 3′ downstream region. IRF5 may assist in formation of a high order enhancer structure linking together the regulatory regions in the TNF 5′ upstream and 3′ downstream and allowing for maintaining of transcription over a longer time (FIG. 29). Based on the resistance of IRF5^{-/-} mice to lethal endotoxic shock, impaired production of pro-inflammatory cytokines and deficiency in Th1 immune response, IRF5 was proposed as a target for therapeutic interventions⁸. Here we define RelA-IRF5 interactions as a putative target for cell-specific modulation of TNF expression, and possible other selected inflammatory mediators.

### REFERENCES FOR EXAMPLE 2

1. Jongeneel C V. Regulation of the TNF alpha gene. Prog Clin Biol Res. 1994; 388:367-381.
2. Collart M A, Baeuerle P, Vassalli P. Regulation of tumor necrosis factor alpha transcription in macrophages: involvement of four kappa B-like motifs and of constitutive and inducible forms of NF-kappa B. Mol Cell Biol. 1990; 10:1498-1506.
3. Shakhov A N, Collart M A, Vassalli P, Nedospasov S A, Jongeneel C V. Kappa B-type enhancers are involved in lipopolysaccharide-mediated transcriptional activation of the tumor necrosis factor alpha gene in primary macrophages. J Exp Med. 1990; 171:35-47.
4. Foxwell B, Browne K, Bondeson J, et al. Efficient adenoviral infection with IkappaB alpha reveals that macrophage tumor necrosis factor alpha production in rheumatoid arthritis is NF-kappaB dependent. Proc Natl Acad Sci USA. 1998; 95:8211-8215.
5. Udalova I A, Knight J C, Vidal V, Nedospasov S A, Kwiatkowski D. Complex NF-kappaB interactions at the distal tumor necrosis factor promoter region in human monocytes. J Biol. Chem. 1998; 273:21178-21186.
6. Honda K, Taniguchi T. Toll-like receptor signaling and IRF transcription factors. IUBMB Life. 2006; 58:290-295.
7. Sakaguchi S, Negishi H, Asagiri M, et al. Essential role of IRF-3 in lipopolysaccharide-induced interferon-beta gene expression and endotoxin shock. Biochem Biophys Res Commun. 2003; 306:860-866.
8. Takaoka A, Yanai H, Kondo S, et al. Integral role of IRF-5 in the gene induction programme activated by Toll-like receptors. Nature. 2005; 434:243-249.
9. Barnes B J, Kellum M J, Field A E, Pitha P M. Multiple regulatory domains of IRF-5 control activation, cellular localization, and induction of chemokines that mediate recruitment of T lymphocytes. Mol Cell Biol. 2002; 22:5721-5740.
10. Chen W, Lam S S, Srinath H, et al. Insights into interferon regulatory factor activation from the crystal structure of dimeric IRF5. Nat Struct Mol. Biol. 2008; 15:1213-1220.
11. Mancl M E, Hu G, Sangster-Guity N, et al. Two discrete promoters regulate the alternatively spliced human interferon regulatory factor-5 isoforms. Multiple isoforms with distinct cell type-specific expression, localization, regulation, and function. J Biol. Chem. 2005; 280:21078-21090.
12. Graham R R, Kozyrev S V, Baechler E C, et al. A common haplotype of interferon regulatory factor 5 (IRF5) regulates splicing and expression and is associated with increased risk of systemic lupus erythematosus. Nat. Genet. 2006; 38:550-555.
13. Dieguez-Gonzalez R, Calaza M, Perez-Pampin E, et al. Association of interferon regulatory factor 5 haplotypes, similar to that found in systemic lupus erythematosus, in a large subgroup of patients with rheumatoid arthritis. Arthritis Rheum. 2008; 58:1264-1274.
14. Han S W, Lee W K, Kwon K T, Lee B K, Nam E J, Kim G W. Association of polymorphisms in interferon regulatory factor 5 gene with rheumatoid arthritis: a metaanalysis. J. Rheumatol. 2009; 36:693-697.
15. Miceli-Richard C, Comets E, Loiseau P, Puechal X, Hachulla E, Mariette X. Association of an IRF5 gene functional polymorphism with Sjogren's syndrome. Arthritis Rheum. 2007; 56:3989-3994.
16. Thomson S J, Goh F G, Banks H, et al. The role of transposable elements in the regulation of IFN-lambdal gene expression. Proc Natl Acad Sci USA. 2009; 106:11564-11569.
17. Fujita M, Kiyono T, Hayashi Y, Ishibashi M. In vivo interaction of human MCM heterohexameric complexes with chromatin. Possible involvement of ATP. J Biol. Chem. 1997; 272:10928-10935.
18. Vlieghe D, Sandelin A, De Bleser P J, et al. A new generation of JASPAR, the open-access repository for transcription factor binding site profiles. Nucleic Acids Res. 2006; 34:D95-97.
19. Grivennikov S I, Tumanov A V, Liepinsh D J, et al. Distinct and nonredundant in vivo functions of TNF produced by t cells and macrophages/neutrophils: protective and deleterious effects. Immunity. 2005; 22:93-104.
20. Abe K, Yarovinsky F O, Murakami T, et al. Distinct contributions of TNF and LT cytokines to the development of dendritic cells in vitro and their recruitment in vivo. Blood. 2003; 101:1477-1483.
21. Ritter U, Meissner A, Ott J, Korner H. Analysis of the maturation process of dendritic cells deficient for TNF and lymphotoxin-alpha reveals an essential role for TNF.
Leukoc Biol. 2003; 74:216-222.
22. Aspalter R M, Eibl M M, Wolf H M. Regulation of TCR-mediated T cell activation by TNF-RII. J Leukoc Biol. 2003; 74:572-582.
23. Yoshimura S, Bondeson J, Foxwell B M, Brennan F M, Feldmann M. Effective antigen presentation by dendritic cells is NF-kappaB dependent: coordinate regulation of MHC, co-stimulatory molecules and cytokines. Int Immunol. 2001; 13:675-683.
24. Kuprash D V, Udalova I A, Turetskaya R L, Kwiatkowski D, Rice N R, Nedospasov S A. Similarities and differences between human and murine TNF promoters in their response to lipopolysaccharide. J. Immunol. 1999; 162:4045-4052.
25. Kuprash D V, Udalova I A, Turetskaya R L, Rice N R, Nedospasov S A. Conserved kappa B element located downstream of the tumor necrosis factor alpha gene: distinct NF-kappa B binding pattern and enhancer activity in LPS activated murine macrophages. Oncogene. 1995; 11:97-106.
26. Schoenemeyer A, Barnes B J, Manci M E, et al. The interferon regulatory factor, IRF5, is a central mediator of toll-like receptor 7 signaling. J Biol. Chem. 2005; 280:17005-17012.
27. Yang L, Zhao T, Shi X, et al. Functional analysis of a dominant negative mutation of interferon regulatory factor 5. PLoS One. 2009; 4:e5500.
28. Neumann M, Fries H, Scheicher C, et al. Differential expression of Rel/NF-kappaB and octamer factors is a hallmark of the generation and maturation of dendritic cells. Blood. 2000; 95:277-285.
29. Saccani S, Pantano S, Natoli G. Modulation of NF-kappaB activity by exchange of dimers. Mol. Cell. 2003; 11:1563-1574.
30. Ramirez-Carrozzi V R, Nazarian A A, Li C C, et al. Selective and antagonistic functions of SWI/SNF and Mi-2beta nucleosome remodeling complexes during an inflammatory response. Genes Dev. 2006; 20:282-296.
31. Hargreaves D C, Horng T, Medzhitov R. Control of inducible gene expression by signal-dependent transcriptional elongation. Cell. 2009; 138:129-145.
32. Ramirez-Carrozzi V R, Braas D, Bhatt D M, et al. A unifying model for the selective regulation of inducible transcription by CpG islands and nucleosome remodeling. Cell. 2009; 138:114-128.
33. Ogawa S, Lozach J, Benner C, et al. Molecular determinants of crosstalk between nuclear receptors and toll-like receptors. Cell. 2005; 122:707-721.
34. Tsytsykova A V, Rajsbaum R, Falvo J V, Ligeiro F, Neely S R, Goldfeld A E. Activation-dependent intrachromosomal interactions formed by the TNF gene promoter and two distal enhancers. Proc Natl Acad Sci USA. 2007; 104:16850-16855.
35. Taylor J M, Wicks K, Vandiedonck C, Knight J C. Chromatin profiling across the human tumour necrosis factor gene locus reveals a complex, cell type-specific landscape with novel regulatory elements. Nucleic Acids Res. 2008; 36:4845-4862.
36. Ueno H, Klechevsky E, Morita R, et al. Dendritic cell subsets in health and disease. Immunol Rev. 2007; 219:118-142.
37. Baldwin H M, Ito-Ihara T, Isaacs J D, Hilkens C M. TNF{alpha} blockade impairs dendritic cell survival and function in rheumatoid arthritis. Ann Rheum Dis. 2009.
38. Glaros T, Larsen M, Li L. Macrophages and fibroblasts during inflammation, tissue damage and organ injury. Front Biosci. 2009; 14:3988-3993.
39. Cheng T F, Brzostek S, Ando O, Van Scoy S, Kumar K P, Reich N C. Differential activation of IFN regulatory factor (IRF)-3 and IRF-5 transcription factors during viral infection. J. Immunol. 2006; 176:7462-7470.
40. Paun A, Reined J T, Jiang Z, et al. Functional characterization of murine interferon regulatory factor 5 (IRF-5) and its role in the innate antiviral response. J Biol. Chem. 2008; 283:14295-14308.
41. Negishi H, Ohba Y, Yanai H, et al. Negative regulation of Toll-like-receptor signaling by IRF-4. Proc Natl Acad Sci USA. 2005; 102:15989-15994.
42. Tamura T, Tailor P, Yamaoka K, et al. IFN regulatory factor-4 and -8 govern dendritic cell subset development and their functional diversity. J. Immunol. 2005; 174:2573-2581.
43. Lehtonen A, Veckman V, Nikula T, et al. Differential expression of IFN regulatory factor 4 gene in human monocyte-derived dendritic cells and macrophages. J. Immunol. 2005; 175:6570-6579.

### EXAMPLE 3

### Mapping the Irf5-RELA Interaction Interface

### Methods

As shown schematically in FIG. 30, One-Strep affinity purification utilises One-Strep tag fusion proteins as bait within the cell to form protein complexes, which can then be isolated from cell lysates by exposure to Streptactin-coated beads, and retrieved by competitive elution by biotin. We used this method to map the IRF5-RELA interaction interface using IRF5 mutants. The particular IRF5 mutants that we used are shown in FIG. 31, and the RelA mutants are shown in FIG. 33.

The IRF5 truncated mutants were co-transfected with Flag-tagged RelA into HEK293-TLR4-CD14/Md2 cells, and the flag-tagged RelA truncated mutants were co-transfected with full-length IRF5. The resulting cell lysates were utilised for One-Strep affinity purification. Bait proteins were visualised in the input lysates and eluates by Western blotting using a Strep-tag specific antibody, and RelA-Flag was visualised using a Flag-tag specific antibody (shown in FIG. 32 for IRF5 mutants and FIG. 33 for RelA mutants).

### Results and Conclusions

IRF5 recruitment to a 3′ downstream region of the TNFα gene locus is dependent on protein-protein interaction with RelA. We show by One-Strep affinity purification that IRF5 and RelA are able to physically interact, and that the IRF Association Domain (IAD) of IRF5 and the Dimerisation Domain (DD) of RelA are part of the interaction interface. We believe that via this interaction, IRF5 is able to modulate TNFα gene expression, and blocking this interaction will affect IRF5 activity.

### EXAMPLE 4

### Identifying TRIM28 as an IRF5 Interacting Protein

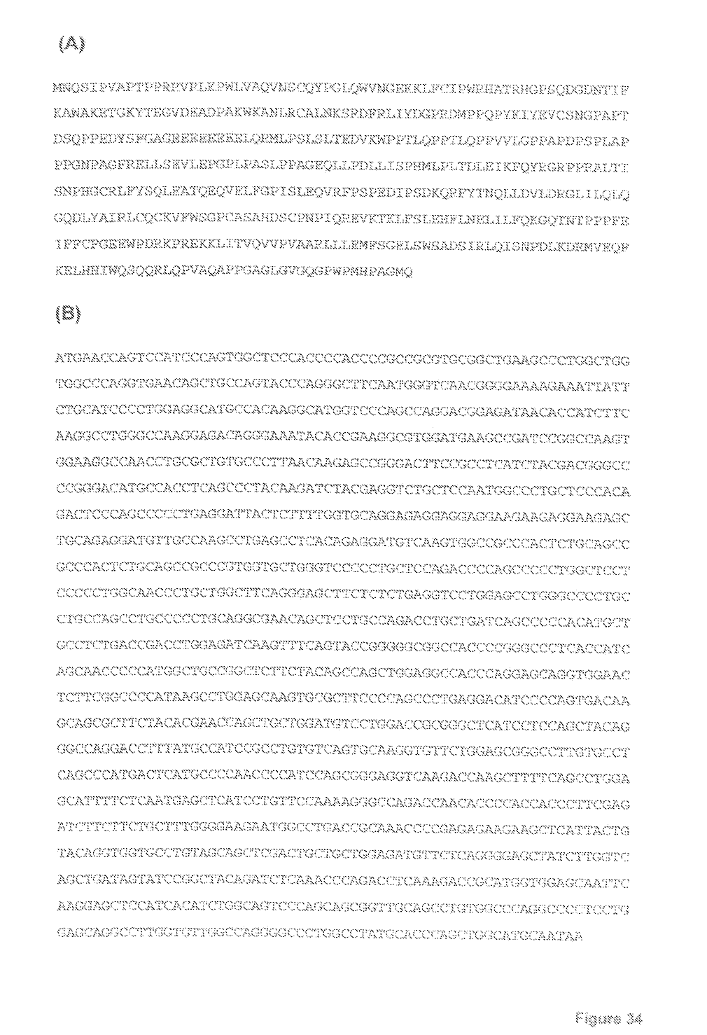

1×10⁸ HEK293-TLR4-CD14/Md2 cells were infected by adenovirus containing the One-Strep-IRF5 fusion protein or the One-Strep tag alone (negative control for protein-protein interactions) for 2 hours serum-free at an MOI of 5. After 24 hours, cells were stimulated +/−LPS at 500 ng/ml for 2 hours, before cross-linking with DTBP and lysis with Farnham cytoplasmic lysis buffer containing protease inhibitors. The resulting nuclear pellets were lysed with RIPA lysis buffer containing inhibitors, and sonicated for 8 minutes in 30s pulses to disrupt chromatin. Cytoplasmic and nuclear lysates were subjected to One-Strep affinity purification, and the eluates concentrated to 50 μl by Vivaspin column. Concentrated eluates were run on SDS-PAGE gel and colloidal blue-stained to visualise bait and prey proteins (see FIG. 35). Interesting bands were excised and trypsinised for identification by mass spectrometry, as a result of which TRIM28 was identified.

We then validated the IRF5-TRIM28 interaction using the same buffers but no cross-linking. 4×10⁷ HEK293-TLR4-CD14/Md2 cells were infected by adenovirus containing the One-Strep-IRF5 fusion protein or the One-Strep tag alone (negative control for protein-protein interactions) for 2 hours serum-free at an MOI of 5. After 24 hours, cells were harvested and lysed with Farnham cytoplasmic lysis buffer containing protease inhibitors. The resulting nuclear pellets were lysed with RIPA lysis buffer containing inhibitors, and sonicated for 8 minutes in 30s pulses to disrupt chromatin. Cytoplasmic and nuclear lysates were subjected to One-Strep affinity purification, and the eluates concentrated to 50 μl by Vivaspin column. Concentrated eluates were run on SDS-PAGE gel and transferred to PVDF membrane for Western blotting with a TRIM28 specific antibody. As shown in FIG. 36, use of the TRIM28 specific antibody confirmed that TRIM28 is an IRF5 interacting protein.

1. A method of treating a patient having an autoimmune disease, or a Th1 polarising infection, or a condition associated with inflammation other than asthma or allergy, the method comprising administering to the patient a therapeutically effective amount of an inhibitor of Interferon Regulatory Factor 5 (IRF5).

2. A method according to claim 1 wherein the autoimmune disease is selected from the group consisting of Crohn's disease, systemic lupus erythematosus (SLE), psoriasis, rheumatoid arthritis (RA), multiple sclerosis (MS), Sjogren's syndrome, inflammatory bowel disease (IBD) and atherosclerosis.

3. A method according to claim 1 wherein the Th1 polarising infection is a bacterial infection or a viral infection.

4. A method according to claim 1 wherein the condition associated with inflammation other than asthma or allergy is a condition associated with chronic inflammation, such as transplant rejection, or is a condition associated with acute inflammation, such as a response to injury or an ulcer.

5. A method according to claim 1 wherein the inhibitor of IRF5 is selected from the group consisting of a polynucleotide inhibitor of IRF5, a competitive inhibitor of IRF5, an agent that inhibits the expression of IRF5 in cells of the macrophage/monocyte lineage, a molecule that interferes with the IRF5-RelA interaction, and a dominant-negative mutant of IRF5.

6. A method according to claim 5 wherein the polynucleotide inhibitor of IRF5 is an siRNA, antisense nucleic acid or ribozyme molecule that targets IRF5.

7. A method according to claim 6 wherein administering the polynucleotide inhibitor of IRF5 comprises administering a nucleic acid molecule that encodes it.

8. A method according to claim 5 wherein the agent that inhibits the expression of IRF5 in cells of the macrophage/monocyte lineage is macrophage-colony stimulating factor (M-CSF) or an M-CSF receptor agonist.

9. A method according to claim 5 wherein the competitive inhibitor of IRF5 is IRF4.

10. A method according to claim 5 wherein the molecule that interferes with the IRF5-RelA interaction is a mutant of IRF5 which has a mutated or deleted IRF interaction domain (IAD).

11. A method according to claim 5 wherein the dominant-negative mutant of IRF5 has a mutated or deleted DNA binding domain (DBD).

12. A method of treating a patient having a condition selected from a compromised immune system, a Th2 polarising infection, and cancer, the method comprising administering to the patient a therapeutically effective amount of IRF5, or an agonist of IRF5, or an agent that induces the expression of IRF5 in cells of the macrophage/monocyte lineage.

13. A method according to claim 12 wherein the Th2 polarising infection is a parasitic infection.

14. A method according to claim 12 wherein the Th2 polarising infection is an infection with an organism selected from the group consisting of a helminth, a flatworm, a roundworm, Leishmania major, Trypanosoma brucei, Neisseria meningitides, a Candida, and a Cryptococcus.

15. A method according to claim 12 wherein the agent that induces the expression of IRF5 in cells of the macrophage/monocyte lineage is selected from the group consisting of granulocyte macrophage-colony stimulating factor (GM-CSF), a GM-CSF receptor agonist, IFN-γ and IL-23.

16. (canceled)

17. A method according to claim 12 wherein the cancer is liver, breast, colon, lung, prostate, pancreas or skin cancer, or is lymphoma or leukaemia.

18. A method according to claim 12 wherein administering the therapeutically effective amount of IRF5 comprises administering a nucleic acid molecule that encodes IRF5.

19. A method according to claim 18 wherein the nucleic acid molecule that encodes IRF5 is administered via a viral vector, for example an adenoviral vector.

20.-44. (canceled)

45. A method of identifying an inhibitor of IRF5, the method comprising:
providing IRF5 (SEQ ID No: 1) or a portion or a variant thereof, said portion or variant of IRF5 being capable of binding to:
a) full-length RelA (SEQ ID No: 7), or a portion or a variant thereof, said portion or variant of RelA being capable of binding to full-length IRF5 (SEQ ID No: 1); or
b) full-length TRIM28 (SEQ ID No: 9), or a portion or a variant thereof, said portion or variant of TRIM28 being capable of binding to full-length IRF5 (SEQ ID No: 1);
providing a test agent; and
assessing the binding of IRF5 or said portion or a variant thereof with RelA or said portion or a variant of RelA, or with TRIM28 or said portion or a variant of TRIM28, in the presence of the test agent,
wherein a test agent that interferes with IRF5/RelA binding or IRF5/TRIM28 binding may be an inhibitor of IRF5.

46.-49. (canceled)

50. The method of claim 45, further comprising:
determining whether the test agent inhibits at least one function or activity of IRF5 selected from the group consisting of:
inhibition of IRF5-mediated expression and/or secretion of TNF from DCs;
inhibition or reversal of IRF5-mediated inhibition of expression and/or secretion of IL-10 from cells of the macrophage/monocyte lineage;
inhibition of IRF-mediated upregulation of expression of one or more genes selected from the group consisting of CXCR3, CXCR4, CXCR5, CXCR7, EBI3, TNFSF4, TNFSF9, LTA, LTB, IFN-gamma, CCL1, CCL3, CXCL5, IL-19 and IL-32 in cells of the macrophage/monocyte lineage;
inhibition of IRF-mediated downregulation of expression of one or more genes selected from the group consisting of CSF1R, IL-1R2, IL1RA and TGFβ in cells of the macrophage/monocyte lineage; or
inhibition or reversal of the IRF5-mediated polarisation of cells of the macrophage/monocyte lineage towards the macrophage M1 phenotype,
wherein a test agent that inhibits at least one function or activity of IRF5 may be an inhibitor of IRF5.

51.-67. (canceled)

68. A method of identifying an inducer of IRF5 expression, the method comprising:
providing a test agent;
providing a reporter gene operably linked to an IRF5 promoter;
determining whether the test agent induces the expression of the reporter gene; and
determining whether a test agent that induces the expression of the reporter gene induces one or more functions or activities of IRF5 selected from the group consisting of:
IRF5-mediated expression and/or secretion of TNF from DCs;
IRF5-mediated inhibition of expression and/or secretion of IL-10 from cells of the macrophage/monocyte lineage;
IRF-mediated upregulation of expression of one or more genes selected from the group consisting of CXCR3, CXCR4, CXCR5, CXCR7, EBI3, TNFSF4, TNFSF9, LTA, LTB, IFN-gamma, CCL1, CCL3, CXCL5, IL-19 and IL-32 in cells of the macrophage/monocyte lineage;
IRF-mediated downregulation of expression of one or more genes selected from the group consisting of CSF1R, IL-1R2, IL1RA and TGFβ in cells of the macrophage/monocyte lineage; and
polarises cells of the macrophage/monocyte lineage towards the macrophage M1 phenotype.

69. (canceled)

70. (canceled)


SEQUENCE LISTING (73 sequences)

SEQ ID NO: 1 - protein, 488 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTEDVKWPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 2 - DNA, 1467 bp, Homo sapiens
atgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggcctgggccaaggagacagggaaatacaccgaaggcgtggatgaagccgatccggccaagtggaaggccaacctgcgctgtgcccttaacaagagccgggacttccgcctcatctacgacgggccccgggacatgccacctcagccctacaagatctacgaggtctgctccaatggccctgctcccacagactcccagccccctgaggattactcttttggtgcaggagaggaggaggaagaagaggaagagctgcagaggatgttgccaagcctgagcctcacagaggatgtcaagtggccgcccactctgcagccgcccactctgcagccgcccgtggtgctgggtccccctgctccagaccccagccccctggctcctccccctggcaaccctgctggcttcagggagcttctctctgaggtcctggagcctgggcccctgcctgccagcctgccccctgcaggcgaacagctcctgccagacctgctgatcagcccccacatgctgcctctgaccgacctggagatcaagtttcagtaccgggggcggccaccccgggccctcaccatcagcaacccccatggctgccggctcttctacagccagctggaggccacccaggagcaggtggaactcttcggccccataagcctggagcaagtgcgcttccccagccctgaggacatccccagtgacaagcagcgcttctacacgaaccagctgctggatgtcctggaccgcgggctcatcctccagctacagggccaggacctttatgccatccgcctgtgtcagtgcaaggtgttctggagcgggccttgtgcctcagcccatgactcatgccccaaccccatccagcgggaggtcaagaccaagcttttcagcctggagcattttctcaatgagctcatcctgttccaaaagggccagaccaacaccccaccacccttcgagatcttcttctgctttggggaagaatggcctgaccgcaaaccccgagagaagaagctcattactgtacaggtggtgcctgtagcagctcgactgctgctggagatgttctcaggggagctatcttggtcagctgatagtatccggctacagatctcaaacccagacctcaaagaccgcatggtggagcaattcaaggagctccatcacatctggcagtcccagcagcggttgcagcctgtggcccaggcccctcctggagcaggccttggtgttggccaggggccctggcctatgcacccagctggcatgcaataa

SEQ ID NO: 3 - protein, 16 aa, Drosophila melanogaster
RQIKIWFQNRRMKWKK

SEQ ID NO: 4 - protein, 19 aa, Drosophila melanogaster
SGRQIKIWFQNRRMKWKKC

SEQ ID NO: 5 - protein, 14 aa, Human immunodeficiency virus type 1
SGYGRKKRRQRRRC

SEQ ID NO: 6 - protein, 177 aa, Homo sapiens
PAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPD

SEQ ID NO: 7 - protein, 548 aa, Homo sapiens
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 8 - protein, 107 aa, Homo sapiens
PNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDR

SEQ ID NO: 9 - protein, 753 aa, Homo sapiens
MAASAAAASAAAASAASGSPGPGEGSAGGEKRSTAPSAAASASASAAASSPAGGGAEALELLEHCGVCRERLRPEREPRLLPCLHSACSACLGPAAPAAANSSGDGGAAGDGTGPAKSRDGERTVYCNVHKHEPLVLFCESCDTLTCRDCQLNAHKDHQYQFLEDAVRNQRKLLASLVKRLGDKHATLQKSTKEVRSSIRQVSDVQKRVQVDVKMAILQIMKELNKRGRVLVNDAQKVTEGQQERLERQHWTMTKIQKHQEHILRFASWALESDNNTALLLSKKLIYFQLHRALKMIVDPVEPHGEMKFQWDLNAWTKSAEAFGKIVAERPGTNSTGPAPMAPPRAPGPLSKQGSGSSQPMEVQEGYGFGSGDDPYSSAEPHVSGVKRSRSGEGEVSGLMRKVPRVSLERLDLDLTADSQPPVFKVFPGSTTEDYNLIVIERGAAAAATGQPGTAPAGTPGAPPLAGMAIVKEEETEAAIGAPPTATEGPETKPVLMALAEGPGAEGPRLASPSGSTSSGLEVVAPEGTSAPGGGPGTLDDSATICRVCQKPGDLVMCNQCEFCFHLDCHLPALQDVPGEEWSCSLCHVLPDLKEEDGSLSLDGADSTGVVAKLSPANQRKCERVLLALFCHEPCRPLHQLATDSTFSLDQPGGTLDLTLIRARLQEKLSPPYSSPQEFAQDVGRMFKQFNKLTEDKADVQSIIGLQRFFETRMNEAFGDTKFSAVLVEPPPMSLPGAGLSSQELSGGPGDGP

SEQ ID NO: 10 - DNA, 10 bp, Artificial Sequence
  Wild type kB2 site in TNF 5′ upstream
gtgaattccc

SEQ ID NO: 11 - DNA, 10 bp, Artificial Sequence
  Mutated kB2 site in TNF 5′ upstream
ttgaattccc

SEQ ID NO: 12 - DNA, 10 bp, Artificial Sequence
  Wild type kBz site in TNF 5′ upstream
gtgatttcac

SEQ ID NO: 13 - DNA, 10 bp, Artificial Sequence
  Mutated kBz site in TNF 5′ upstream
atcctttcac

SEQ ID NO: 14 - DNA, 10 bp, Artificial Sequence
  Wild type kB2a site in TNF 5′ upstream
gggctgtccc

SEQ ID NO: 15 - DNA, 11 bp, Artificial Sequence
  Mutated kB2a site in TNF 5′ upstream
tagctgtgccc

SEQ ID NO: 16 - DNA, 10 bp, Artificial Sequence
  Wild type kB4 site in TNF 3′ downstream
gggaatttcc

SEQ ID NO: 17 - DNA, 10 bp, Artificial Sequence
  Mutated kB4 site in TNF 3′ downstream
cgcaatgtgc

SEQ ID NO: 18 - DNA, 10 bp, Artificial Sequence
  Wild type kB4a site in TNF 3′ downstream
gggaattcca

SEQ ID NO: 19 - DNA, 10 bp, Artificial Sequence
  Mutated kB4a site in TNF 3′ downstream
cgcaagtgca

SEQ ID NO: 20 - DNA, 20 bp, Homo sapiens
tgagaaggacagggagccaa

SEQ ID NO: 21 - DNA, 22 bp, Homo sapiens
ccacagattttgcaagggatca

SEQ ID NO: 22 - DNA, 21 bp, Artificial Sequence
  IL12-p35 locus primer
tcattttgggccgagctggag

SEQ ID NO: 23 - DNA, 23 bp, Artificial Sequence
  IL12-p35 locus primer
tacatcagcttctcggtgacacg

SEQ ID NO: 24 - DNA, 24 bp, Artificial Sequence
  IL-12p40 locus primer
tccagtaccagcaacagcagcaga

SEQ ID NO: 25 - DNA, 26 bp, Artificial Sequence
  IL-12p40 locus primer
gtaggggcttgggaagtgcttacctt

SEQ ID NO: 26 - DNA, 24 bp, Artificial Sequence
  IL-23p19 locus primer
actgtgaggcctgaaatggggagc

SEQ ID NO: 27 - DNA, 27 bp, Artificial Sequence
  IL-23p19 locus primer
actggatggtcctggtttcatgggaga

SEQ ID NO: 28 - DNA, 27 bp, Artificial Sequence
  IL-10 locus primer
cctgtgccgggaaaccttgattgtggc

SEQ ID NO: 29 - DNA, 27 bp, Artificial Sequence
  IL-10 locus primer
gtcaggaggaccaggcaacagagcagt

SEQ ID NO: 30 - DNA, 26 bp, Artificial Sequence
  kB4 forward oligonucleotide probe
agctgggcatgggaatttccaactct

SEQ ID NO: 31 - DNA, 25 bp, Artificial Sequence
  kB4 reverse oligonucleotide probe
agctgagttggaaattcccatgccc

SEQ ID NO: 32 - DNA, 26 bp, Artificial Sequence
  kB4a forward oligonucleotide probe
agctaactctgggaattccaatcctt

SEQ ID NO: 33 - DNA, 26 bp, Artificial Sequence
  kB4a reverse oligonucleotide probe
agctaaggattggaattcccagagtt

SEQ ID NO: 34 - DNA, 25 bp, Artificial Sequence
  kB4b forward oligonucleotide probe
agctcttgctgggaaaatcctgcag

SEQ ID NO: 35 - DNA, 26 bp, Artificial Sequence
  kB4b reverse oligonucleotide probe
agctgctgcaggattttcccagcaag

SEQ ID NO: 36 - DNA, 29 bp, Artificial Sequence
  ISRE1 forward oligonucleotide probe
agctgaagccaagactgaaaccagcatta

SEQ ID NO: 37 - DNA, 29 bp, Artificial Sequence
  ISRE1 reverse oligonucleotide probe
agcttaatgctggtttcagtcttggcttc

SEQ ID NO: 38 - DNA, 28 bp, Artificial Sequence
  ISRE2 forward oligonucleotide probe
agctccgggtcagaatgaaagaagaagg

SEQ ID NO: 39 - DNA, 29 bp, Artificial Sequence
  ISRE2 reverse oligonucleotide probe
agctccttcttctttcattctgacccggt

SEQ ID NO: 40 - DNA, 29 bp, Artificial Sequence
  ISRE5 forward oligonucleotide probe
agctggagaagaaaccgagacagaaggtg

SEQ ID NO: 41 - DNA, 29 bp, Artificial Sequence
  ISRE5 reverse oligonucleotide probe
agctcaccttctgtctcggtttcttctcc

SEQ ID NO: 42 - DNA, 30 bp, Artificial Sequence
  ′ISRE′16 forward oligonucleotide probe
agcttttgcttagaaaagaaacatggtctc

SEQ ID NO: 43 - DNA, 30 bp, Artificial Sequence
  ′ISRE′16 reverse oligonucleotide probe
agctgagaccatgtttcttttctaagcaaa

SEQ ID NO: 44 - DNA, 32 bp, Artificial Sequence
  ′ISRE′17 forward oligonucleotide probe
agctacataaacaaagcccaacagaatattcc

SEQ ID NO: 45 - DNA, 32 bp, Artificial Sequence
  ′ISRE′17 reverse oligonucleotide probe
agctggaatattctgttgggctttgtttatgt

SEQ ID NO: 46 - DNA, 32 bp, Artificial Sequence
  PRDI-III(IFN-beta) forward oligonucleotide probe
agctgggaaactgaaagggaaagtgaaagtgg

SEQ ID NO: 47 - DNA, 32 bp, Artificial Sequence
  PRDI-III(IFN-beta) reverse oligonucleotide probe
agctccactttcactttccctttcagtttccc

SEQ ID NO: 48 - DNA, 22 bp, Artificial Sequence
  Oligonucleotide probe for TNF locus control region
tgtgtgtctgggagtgagaact

SEQ ID NO: 49 - DNA, 22 bp, Artificial Sequence
  Oligonucleotide probe for TNF locus control region
tcttctcagcttctcctttgct

SEQ ID NO: 50 - DNA, 24 bp, Artificial Sequence
  Oligonucleotide probe for TNF locus region A
ccacagcaatgggtaggagaatgt

SEQ ID NO: 51 - DNA, 23 bp, Artificial Sequence
  Oligonucleotide probe for TNF locus region A
gaggtcctggaggctctttcact

SEQ ID NO: 52 - DNA, 23 bp, Artificial Sequence
  Oligonucleotide probe for TNF locus region B
ggaagccaagactgaaaccagca

SEQ ID NO: 53 - DNA, 22 bp, Artificial Sequence
  Oligonucleotide probe for TNF locus region B
ccgggaattcacagaccccact

SEQ ID NO: 54 - DNA, 20 bp, Artificial Sequence
  Oligonucleotide probe for TNF locus region C
tccctccaaccccgttttct

SEQ ID NO: 55 - DNA, 20 bp, Artificial Sequence
  Oligonucleotide probe for TNF locus region C
taggaccctggaggctgaac

SEQ ID NO: 56 - DNA, 20 bp, Artificial Sequence
  Oligonucleotide probe for TNF locus region D
aactttccaaatccccgccc

SEQ ID NO: 57 - DNA, 20 bp, Artificial Sequence
  Oligonucleotide probe for TNF locus region D
ggtgtgccaacaactgcctt

SEQ ID NO: 58 - DNA, 20 bp, Artificial Sequence
  Oligonucleotide probe for TNF locus region E
cagcaaggacagcagaggac

SEQ ID NO: 59 - DNA, 20 bp, Artificial Sequence
  Oligonucleotide probe for TNF locus region E
tcccggatcatgctttcagt

SEQ ID NO: 60 - DNA, 22 bp, Artificial Sequence
  Oligonucleotide probe for TNF locus region F
ggcagtcagtaagtgtctccaa

SEQ ID NO: 61 - DNA, 22 bp, Artificial Sequence
  Oligonucleotide probe for TNF locus region F
tacctacaacatgggctacagg

SEQ ID NO: 62 - DNA, 22 bp, Artificial Sequence
  Oligonucleotide probe for TNF locus region G
acagctttgatccctgacatct

SEQ ID NO: 63 - DNA, 22 bp, Artificial Sequence
  Oligonucleotide probe for TNF locus region G
ctccgtgtctcaaggaagtctg

SEQ ID NO: 64 - DNA, 24 bp, Artificial Sequence
  Oligonucleotide probe for TNF locus region H
atattccccatcccccaggaaaca

SEQ ID NO: 65 - DNA, 23 bp, Artificial Sequence
  Oligonucleotide probe for TNF locus region H
ctgcaacagccggaaatctcacc

SEQ ID NO: 66 - DNA, 20 bp, Artificial Sequence
  Oligonucleotide probe for TNF locus region I
gaggacctcactcagccctt

SEQ ID NO: 67 - DNA, 20 bp, Artificial Sequence
  Oligonucleotide probe for TNF locus region I
cggcagttcggttccttgtt

SEQ ID NO: 68 - DNA, 22 bp, Artificial Sequence
  Oligonucleotide probe for TNF locus region J
actggtctttgtggtgaaggag

SEQ ID NO: 69 - DNA, 22 bp, Artificial Sequence
  Oligonucleotide probe for TNF locus region J
gaactagtgggctcaagtggtc

SEQ ID NO: 70 - DNA, 23 bp, Artificial Sequence
  Oligonucleotide probe for TNF locus region K
gctatgatcatgccactgtaccc

SEQ ID NO: 71 - DNA, 22 bp, Artificial Sequence
  Oligonucleotide probe for TNF locus region K
taccacatggttttctcctgcc

SEQ ID NO: 72 - DNA, 22 bp, Artificial Sequence
  Oligonucleotide probe for TNF locus region L
gctgaaagtcagccatgaagta

SEQ ID NO: 73 - DNA, 22 bp, Artificial Sequence
  Oligonucleotide probe for TNF locus region L
cacttagggtgtcccatttagg